(19) United States
(12) Patent Application Publication
(10) Pub. No.: US 2023/0382919 A1
(43) Pub. Date: Nov. 30, 2023

(54) OXA- IBOGAINE INSPIRED ANALOGUES FOR TREATMENT OF NEUROLOGICAL AND PSYCHIATRIC DISORDERS

(71) Applicant: The Trustees of Columbia University in the City of New York, New York, NY (US)

(72) Inventors: Dalibor Sames, New York, NY (US); Vaclav Havel, Opava (CZ); Benjamin Bechond, New York, NY (US); David Lankri, Beit Shean (IL)

(73) Assignee: The Trustees of Columbia University in the City of New York, New York, NY (US)

(21) Appl. No.: 18/366,544

(22) Filed: Aug. 7, 2023

Related U.S. Application Data
(63) Continuation of application No. PCT/US2022/015681, filed on Feb. 8, 2022.
(60) Provisional application No. 63/147,157, filed on Feb. 8, 2021.

(51) Int. Cl. C07D 487/18; C07D 487/04; C07D 491/18; C07D 491/048; C07D 495/04; C07D 495/18

(52) U.S. Cl. CPC C07D 487/18; C07D 487/04; C07D 491/18; C07D 491/048; C07D 495/04; C07D 495/18

(57) ABSTRACT

The present invention provides a compound having the structure:

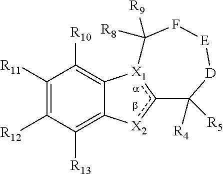

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,
   or a pharmaceutically acceptable salt thereof.

This application claims priority of U.S. Provisional Application No. 63/147,157, filed Feb. 8, 2021, the contents of which are hereby incorporated by reference.

This invention was made with government support under R01DA050613 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Ibogaine is the major psychoactive alkaloid found in the root bark of Tabernanthe iboga, a plant native to West Central Africa (Alper, K. R. 2001). The root bark has been used as a religious and healing sacrament by the native people in Africa owing to its distinct psychedelic effects. The clinical claims of ibogaine's anti-addictive properties, discovered in the U.S. in the 1960's, have largely been recapitulated in animal models of substance use disorders (SUDs), where ibogaine and its main metabolite, noribogaine, show a plethora of effects relevant to different aspects of SUDs (Glick, S. D. et al. 2001; Belgers, M. et al. 2016; Mash, D. C. et al. 2016).

SUDs are psychiatric disorders that affect nearly 20 million adults in the US. Unfortunately, limited treatment options are currently available to these patients. Considering the large unmet needs in SUDs and psychiatric disorders in general, there is a strong impetus to develop new analogs that increase ibogaine's safety and therapeutic index for the treatment of such diseases. Additionally, there is a need for new compounds that can be used to study biological mechanisms that underpin ibogaine's effects and enhance our understanding of ibogaine's mechanism of action.

The present invention represents novel ibogaine analogs of compounds previously disclosed (U.S. Pat. No. 9,988,377; U.S. application Ser. Nos. 14/240,681, 15/528,339; PCT International Application No. PCT/US2012/052327, PCT/US2015/062726). These analogs represent a further elaboration and deconstruction of the iboga skeleton to yield simpler and distinct structural systems with distinct pharmacology as well as improved side effects. The compounds described herein may be useful in treating opioid use disorder (OUD) and other SUDs, mood disorders, depression, and anxiety disorders, migraine and cluster headaches.

### SUMMARY OF THE INVENTION

The present invention provides a compound having the structure:

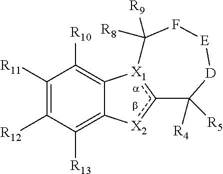

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

or a pharmaceutically acceptable salt thereof.

The present invention also provides a pharmaceutical composition comprising a compound having the structure:

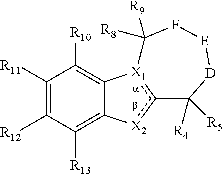

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

or a pharmaceutically acceptable salt thereof.

The present invention also provides a method of treating a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy, comprising administering to the subject an effective amount of a compound having the structure:

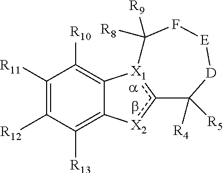

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,

or a pharmaceutically acceptable salt thereof, so as to thereby treat a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy.

### BRIEF DESCRIPTION OF THE FIGURES

FIG. 1. A/ Tail-flick test performed after administration of compound 61. B/ Detailed examination of 10-30 mg/kg dose range, determined ED₅₀=16.51 mg/kg.

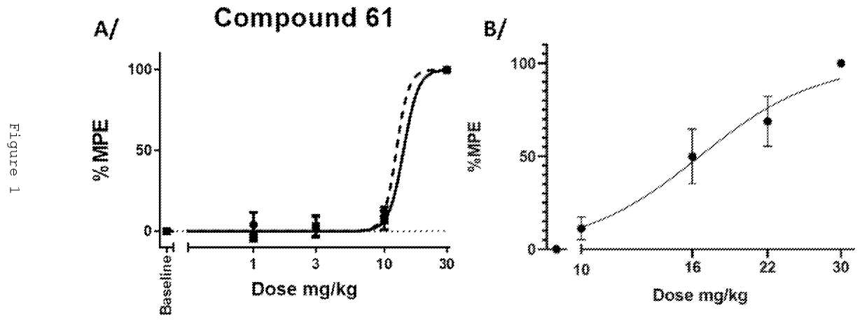

### DETAILED DESCRIPTION OF THE INVENTION

The present invention provides a compound having the structure:

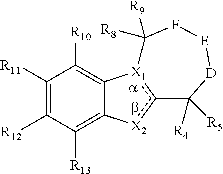

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

or a pharmaceutically acceptable salt thereof.

The present invention also provides a compound having the structure:

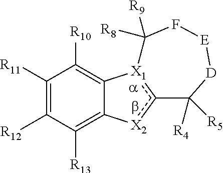

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and Ru is Br, then D and E is other than NH, wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and Ru is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then Ru is other than H, F or —CH₃,

or a pharmaceutically acceptable salt thereof.

In some embodiments of the above compound,
wherein
X₁ is C or N;
X₂ is O, S, N or CR₁₅,
   wherein R₁₅ is H, -(alkyl) or -cycloalkyl;
α and β represent a bond that is present or absent, and wherein either α or β is present,
   wherein when α is present, then X₁ is C and X₂ is O or S, or
   when β is present, then X₁ is N and X₂ is N or CR₁₅;
R₁ is H, or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁, R₁₁, R₁₂ or R₁₃ is other than H and CH₃, and R₁₁ is other than a ketone and a carboxylic acid,

or a pharmaceutically acceptable salt thereof.

In some embodiments of the above compound,
wherein
X₁ is C or N;
X₂ is O, S or CR₁₅,
   wherein R₁₅ is H, -(alkyl) or -cycloalkyl;
α and β represent a bond that is present or absent, and wherein either α or β is present,
   wherein when α is present, then X₁ is C and X₂ is O or S, or
   when β is present, then X₁ is N and X₂ is CR₁₅;
R₁ is H or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and Ru is other than a ketone and a carboxylic acid,

or a pharmaceutically acceptable salt thereof.

In some embodiments of the above compound,
wherein
X₁ is C;
X₂ is O or S;
R, is H or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,

or a pharmaceutically acceptable salt thereof.

In some embodiments of the above compound,
wherein
X₁ is C or N;
X₂ is N or NR₁₄,
   wherein R₁₄ is H, -(alkyl) or -cycloalkyl;
α and β represent a bond that is present or absent, and wherein either α or β is present,
   wherein when α is present, then X₁ is C and X₂ is NR₁₄, or
   when β is present, then X₁ is N and X₂ is N;
R₁ is H, or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

or a pharmaceutically acceptable salt thereof.

In some embodiments of the above compound, wherein
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN.

In some embodiments of the above compound, wherein
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂.

In some embodiments of the above compound, wherein
R₁₀ and R₁₁ together form a —O(CH₂)O— or R₁₁ and R₁₂ together form a —O(CH₂)O— or
R₁₂ and R₁₃ together form a —O(CH₂)O—.

In certain embodiments the compound has the structure below wherein the substituents are defined as in the paragraphs above:

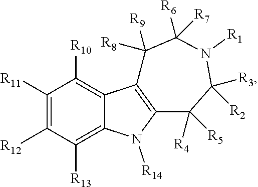

or a pharmaceutically acceptable salt thereof.

In certain embodiments the compound has the structure below wherein the substituents are defined as in the paragraphs above:

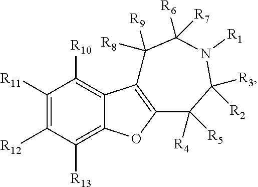

or a pharmaceutically acceptable salt thereof.

In certain embodiments the compound has the structure below wherein the substituents are defined as in the paragraphs above:

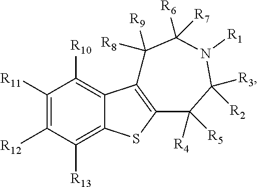

or a pharmaceutically acceptable salt thereof.

In certain embodiments the compound has the structure below wherein the substituents are defined as in the paragraphs above:

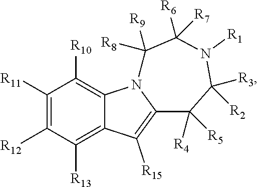

or a pharmaceutically acceptable salt thereof.

In some embodiments, the compound wherein R₁ is H or -(alkyl) In some embodiments, the compound wherein R₁ is H, —CH₃ or —CH₂CH₅.

In some embodiments, the compound wherein R₄, R₅, R₈ and R₉ are each H.

In an embodiment, the compound wherein R₂, R₃, R₆ and R₇ are each independently H, -(alkyl), -alkylcycloalkyl or -alkylaryl.

In some embodiments, the compound wherein R₂, R₃, R₆, and R₇ are each independently H, —CH₃, —CH₂CH₃, —CH₂CH₂CH₃ or —CH(CH₃)₂.

In some embodiments, the compound wherein R₁₀, R₁₁, R₁₂ and R₁₃ are each independently H, -(alkyl), —OH, —O(alkyl), —S(alkyl), —OAc, —CO₂(alkyl), —CF₃ or halogen.

In some embodiments, the compound wherein R₁₀, R₁₁, R₁₂ and R₁₃ are each independently H, —CH₃, —OH, —OCH₃, —SCH₃, —CF₃ or F.

In some embodiments, the compound wherein R₁ is H or —CH₃.

In some embodiments, the compound wherein R₃, R₄, R₅, R₇, R₈ and R₉ are each H.

In an embodiment, the compound wherein R₁ is H or —CH₃, and R₂ and R₆ are each independently H, —CH₃ or —CH₂CH₃.

In some embodiments, the compound wherein R₄, R₅, R₆, R₇, R₈ and R₉ are each H.

In an embodiment, the compound wherein R₂ and R₃ are each independently H, -(alkyl), -alkylcycloalkyl or -alkylaryl.

In some embodiments, the compound wherein R₂ and R₃ are each independently H, —CH₃, —CH₂CH₃, —CH₂CH₂CH₃ or —CH(CH₃)₂.

In some embodiments, the compound wherein R₁₀, R₁₁, R₁₂ and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂, and R₁₃ are each independently H, —CH₃, —OH, —OCH₃, —SCH₃, —CF₃ or F.

In some embodiments, the compound wherein R₁ is H or —CH₃.

In some embodiments, the compound wherein R₁ is H or —CH₃, R₂ is H, —CH₃ or —CH₂CH₃, and R₃ is H.

In some embodiments, the compound wherein R₂, R₃, R₄, R₅, R₈ and R₉ are each H.

In some embodiments, the compound wherein R₆ and R₇ are each independently H, -(alkyl), -alkylcycloalkyl or -alkylaryl.

In some embodiments, the compound wherein R₆ and R₇ are each independently H, —CH₃, —CH₂CH₃, —CH₂CH₂CH₃ or —CH(CH₃)₂.

In some embodiments, the compound wherein R₁₀, R₁₁, R₁₂ and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂ and R₁₃ are each independently H, —CH₃, OH, —OCH₃, —SCH₃, —CF₃ or F.

In some embodiments, the compound wherein R₁ is H or —CH₃.

In some embodiments, the compound wherein R₁ is H or —CH₃, R₆ is H, —CH₃ or —CH₂CH₃, and R₇ is H.

In some embodiments, the compound wherein R₂, R₃, R₄, R₅, R₆, R₇, R₈ and R₉ are each H.

In some embodiments, the compound wherein R₁₀, R₁₁, R₁₂ and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂ and R₁₃ are each independently H, —CH₃, OH, —OCH₃, —SCH₃, —CF₃ or F.

In some embodiments, the compound wherein R₁ is H or —CH₃.

In some embodiments, the compound having the structure:

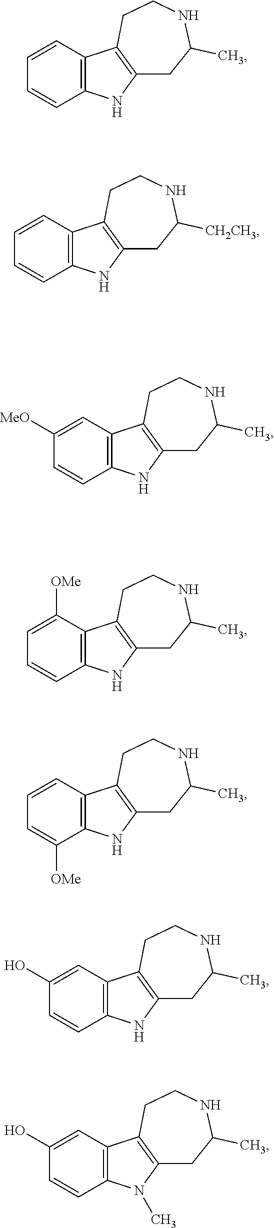

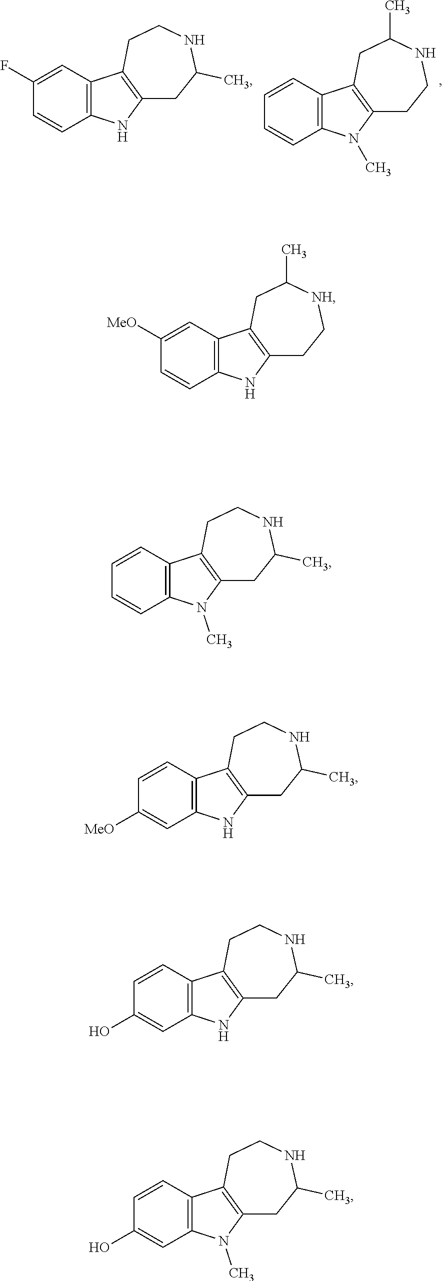

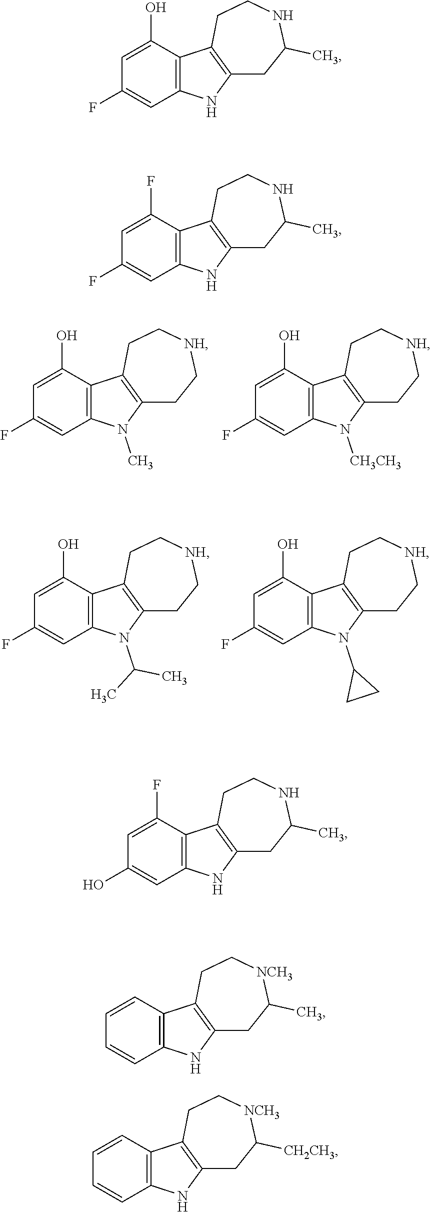

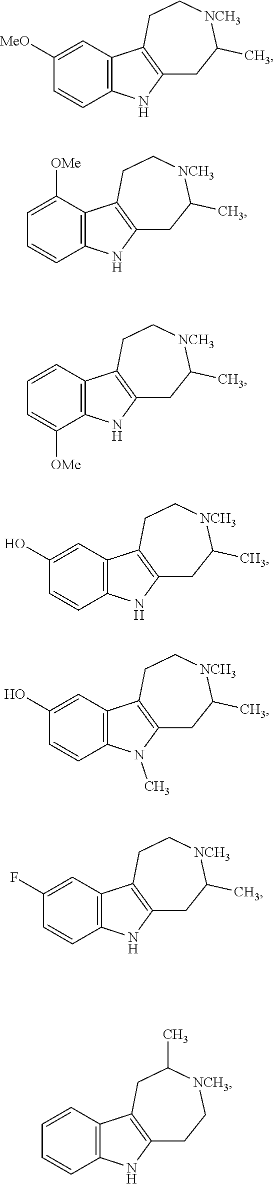

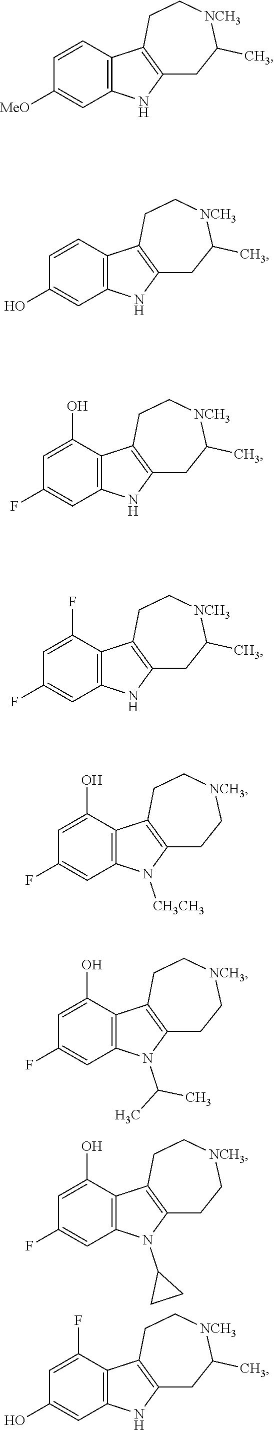

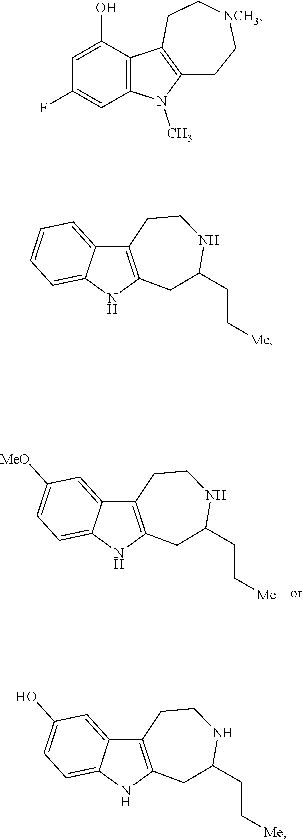 [SMILES: CN1CCc2c(n(C)c3cc(F)cc(O)c23)CC1 ; CCCC1Cc2[nH]c3ccccc3c2CCN1 ; CCCC1Cc2[nH]c3ccc(OC)cc3c2CCN1 ; CCCC1Cc2[nH]c3ccc(O)cc3c2CCN1]

or a pharmaceutically acceptable salt thereof.

In some embodiments, the compound having the structure:

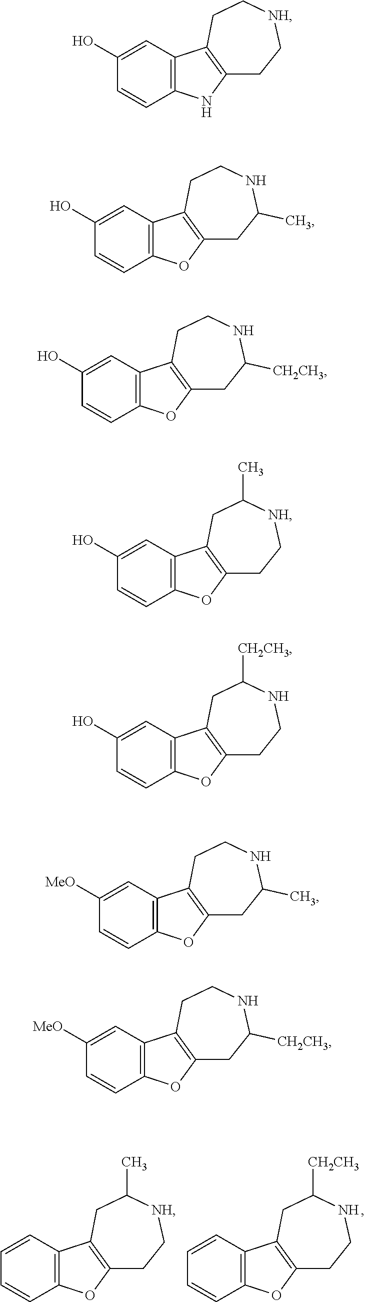

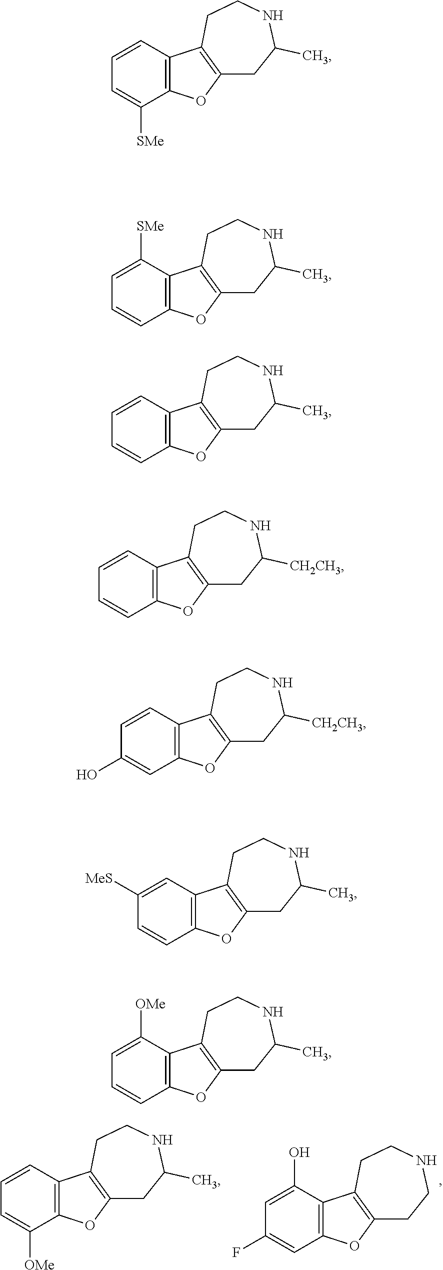

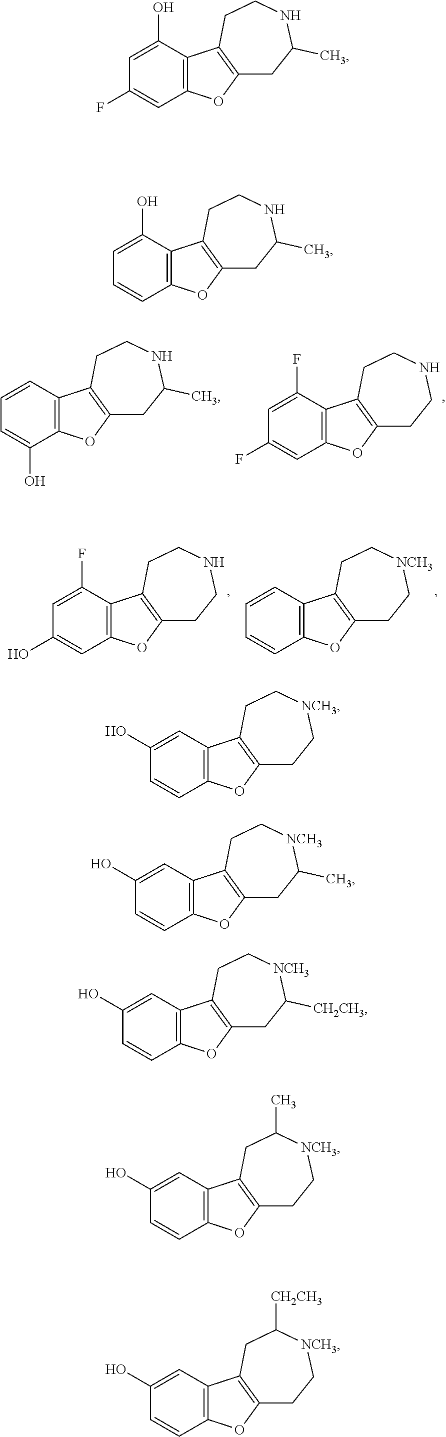

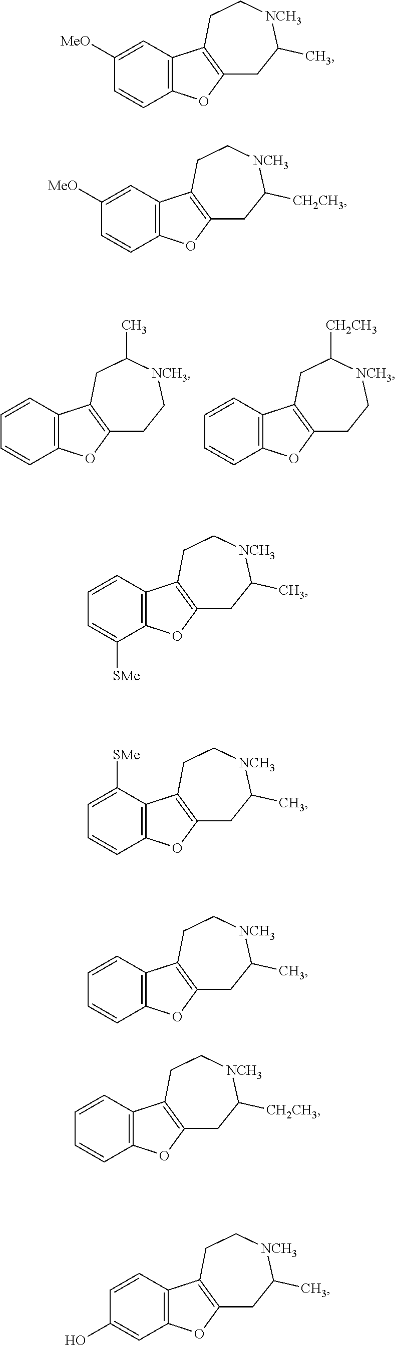

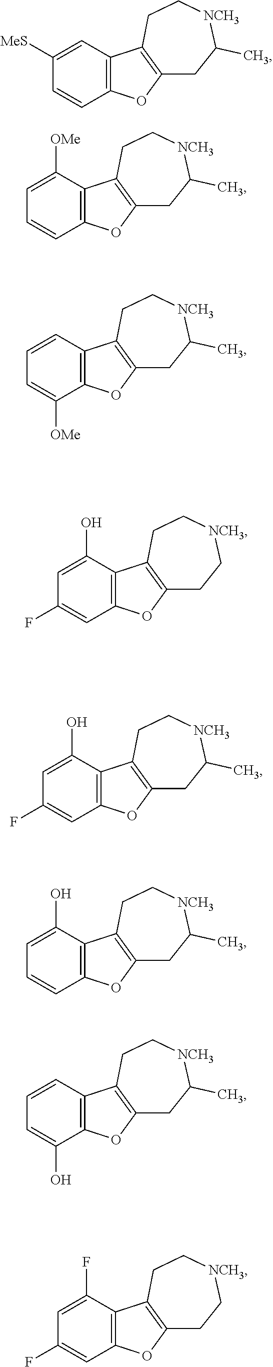

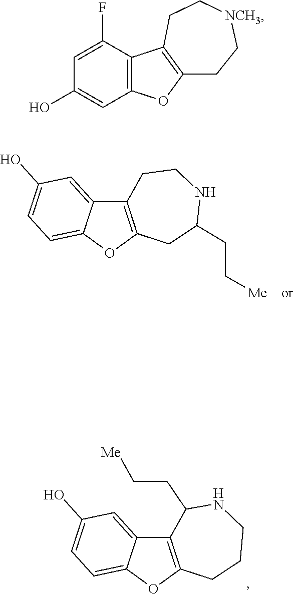 [SMILES: CN1CCc2oc3cc(O)cc(F)c3c2CC1 ; CCCC1Cc2oc3ccc(O)cc3c2CCN1 ; CCCC1NCCCc2oc3ccc(O)cc3c21]

or a pharmaceutically acceptable salt thereof.

In some embodiments, the compound having the structure:

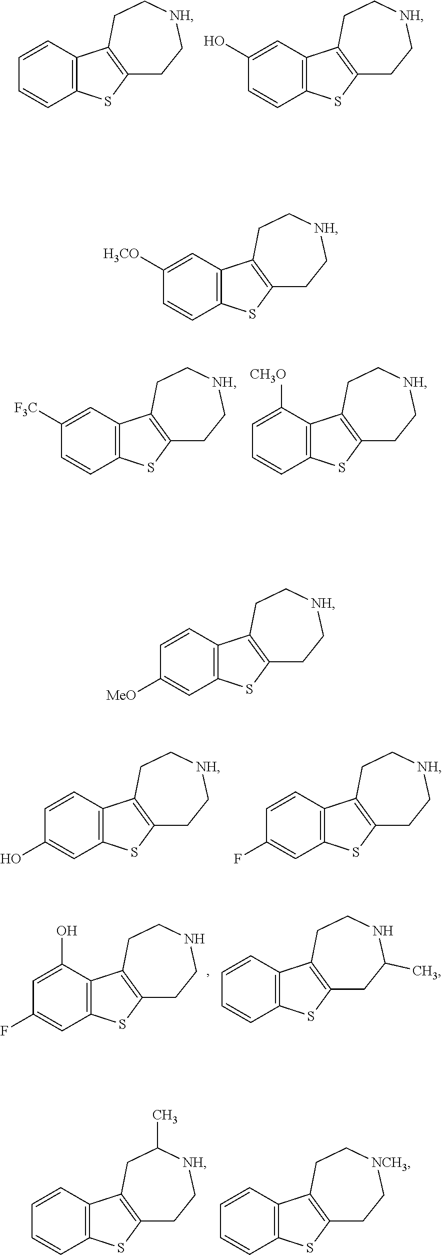

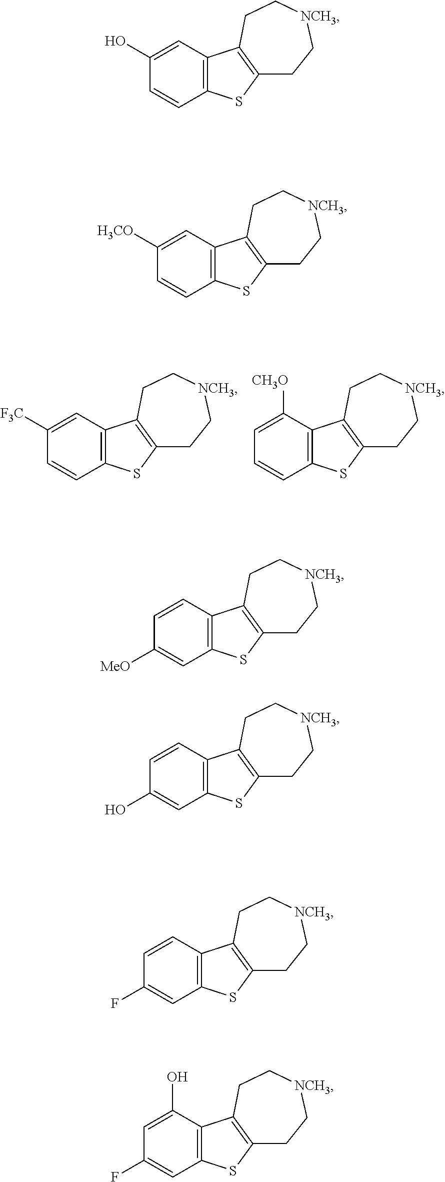

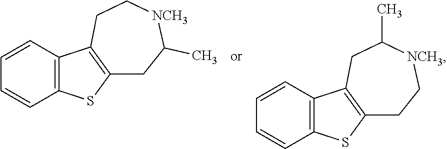 [SMILES: CC1Cc2sc3ccccc3c2CCN1C ; CC1Cc2c(sc3ccccc23)CCN1C]

or a pharmaceutically acceptable salt thereof.

In some embodiments, the compound having the structure:

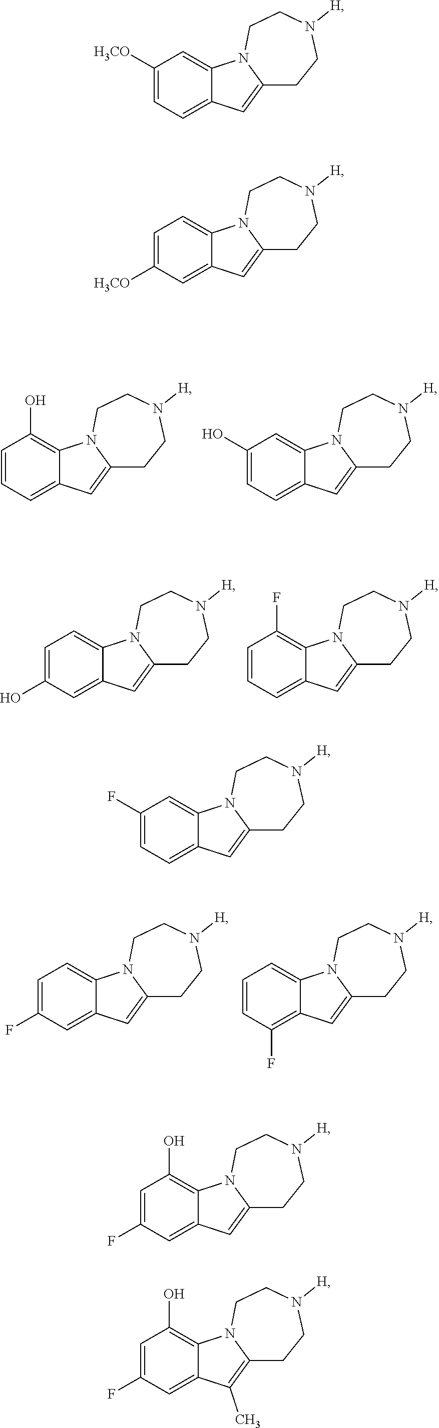

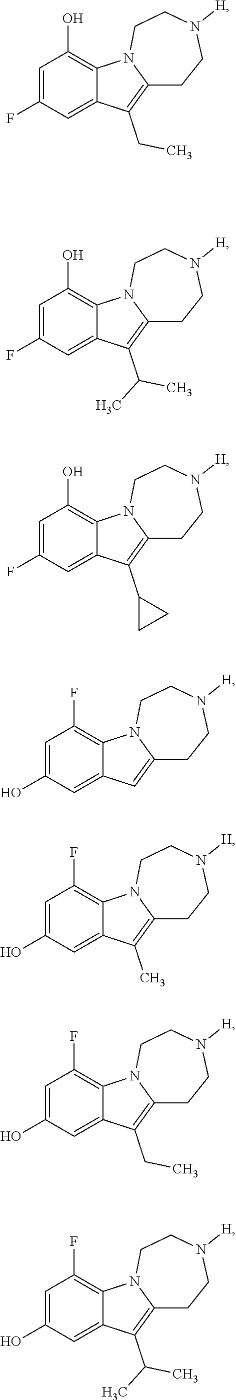

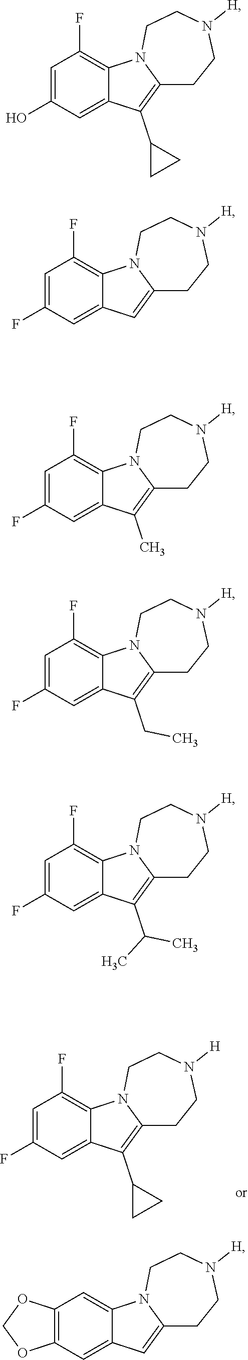

or a pharmaceutically acceptable salt thereof.

In some embodiments, the compound having the structure:

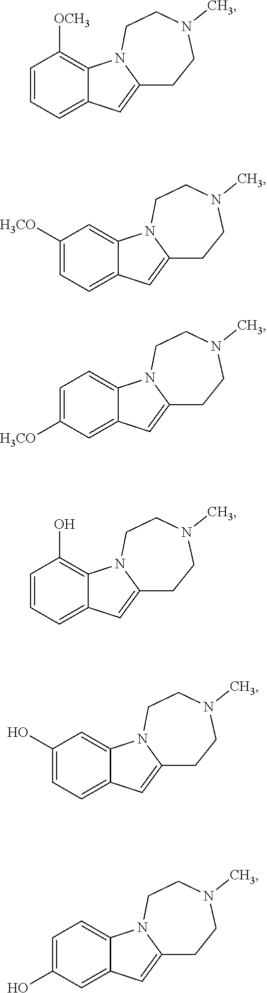 [SMILES: COc1cccc2cc3n(c12)CCN(C)CC3 ; COc1ccc2cc3n(c2c1)CCN(C)CC3 ; COc1ccc2c(c1)cc1n2CCN(C)CC1 ; CN1CCc2cc3cccc(O)c3n2CC1 ; CN1CCc2cc3ccc(O)cc3n2CC1 ; CN1CCc2cc3cc(O)ccc3n2CC1]

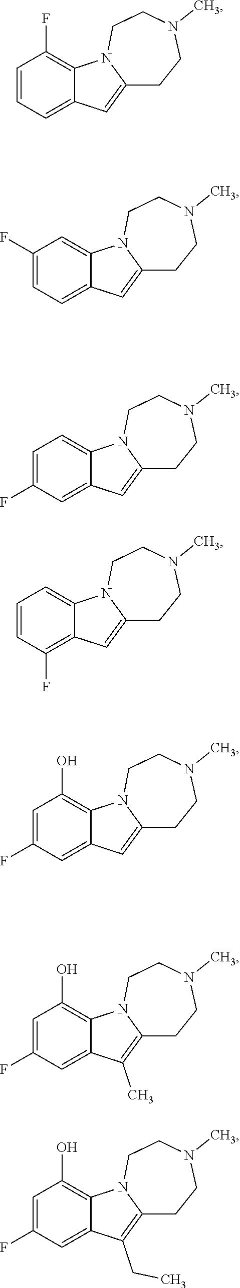

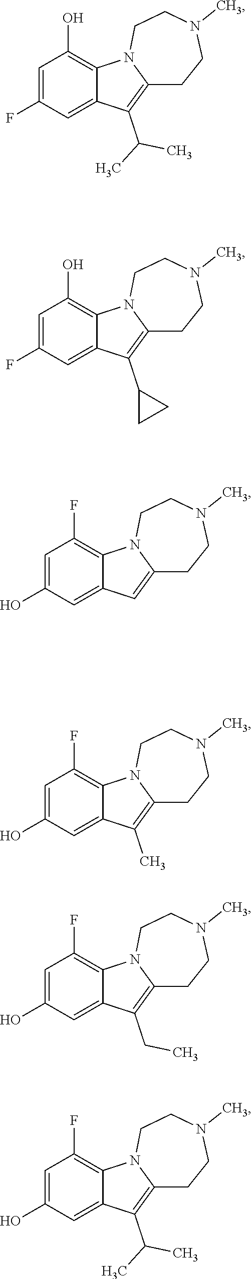 [SMILES: CC(C)c1c2n(c3c(O)cc(F)cc13)CCN(C)CC2 ; CN1CCc2c(C3CC3)c3cc(F)cc(O)c3n2CC1 ; CN1CCc2cc3cc(O)cc(F)c3n2CC1 ; Cc1c2n(c3c(F)cc(O)cc13)CCN(C)CC2 ; CCc1c2n(c3c(F)cc(O)cc13)CCN(C)CC2 ; CC(C)c1c2n(c3c(F)cc(O)cc13)CCN(C)CC2]

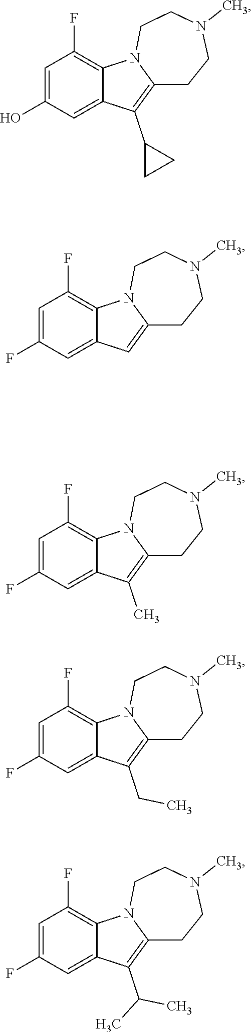 [SMILES: CN1CCc2c(C3CC3)c3cc(O)cc(F)c3n2CC1 ; CN1CCc2cc3cc(F)cc(F)c3n2CC1 ; Cc1c2n(c3c(F)cc(F)cc13)CCN(C)CC2 ; CCc1c2n(c3c(F)cc(F)cc13)CCN(C)CC2 ; CC(C)c1c2n(c3c(F)cc(F)cc13)CCN(C)CC2]

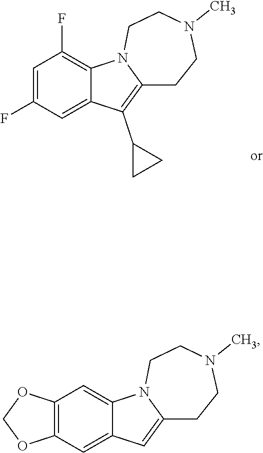 [SMILES: CN1CCc2c(C3CC3)c3cc(F)cc(F)c3n2CC1 ; CN1CCc2cc3cc4c(cc3n2CC1)OCO4]

or a pharmaceutically acceptable salt thereof.

In some embodiments, the compound having the structure:

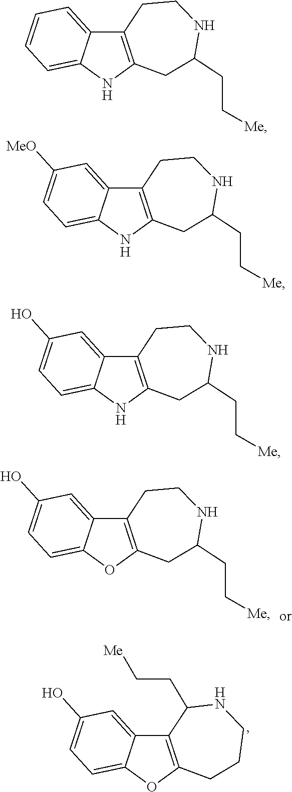 [SMILES: CCCC1Cc2[nH]c3ccccc3c2CCN1 ; CCCC1Cc2[nH]c3ccc(OC)cc3c2CCN1 ; CCCC1Cc2[nH]c3ccc(O)cc3c2CCN1 ; CCCC1Cc2oc3ccc(O)cc3c2CCN1 ; CCCC1NCCCc2oc3ccc(O)cc3c21]

or a pharmaceutically acceptable salt thereof.

In some embodiments, the compound wherein D is CR₂R₃, E is CR₆R₇ and F is NR₁.

In some embodiments, the compound wherein X₁ is C and X₂ is NR₁₄, or X₁ is C and X₂ is O, or X₁ is C and X₂ is S, or X₁ is N and X₂ is CR₁₅, or X₁ is N and X₂ is N.

In certain embodiments the compound has the structure below wherein the substituents are defined as in the paragraphs above:

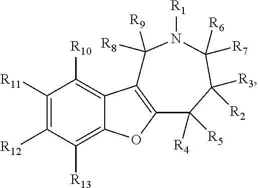

or a pharmaceutically acceptable salt thereof.

In some embodiments, the compound wherein R₁ is H or -(alkyl).

In some embodiments, the compound wherein R₁ is H, —CH₃ or —CH₂CH₅.

In some embodiments, the compound wherein R₆, R₇, R₈ and R₉ are each independently H, -(alkyl), -alkylcycloalkyl or -alkylaryl.

In some embodiments, the compound wherein R₆, R₇, R₈ and R₉ are each independently H, —CH₃, —CH₂CH₃, —CH₂CH₂CH₃ or —CH(CH₃)₂.

In some embodiments, the compound wherein R₂, R₃, R₄, R₅, R₆, R₇, R₈ and R₉ are each H.

In some embodiments, the compound wherein R₁₀, R₁₁, R₁₂ and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂ and R₁₃ are each independently H, —CH₃, OH, —OCH₃, —SCH₃, —CF₃ or F.

In some embodiments, the compound wherein R₁ is H or —CH₃.

In some embodiments, the compound having the structure:

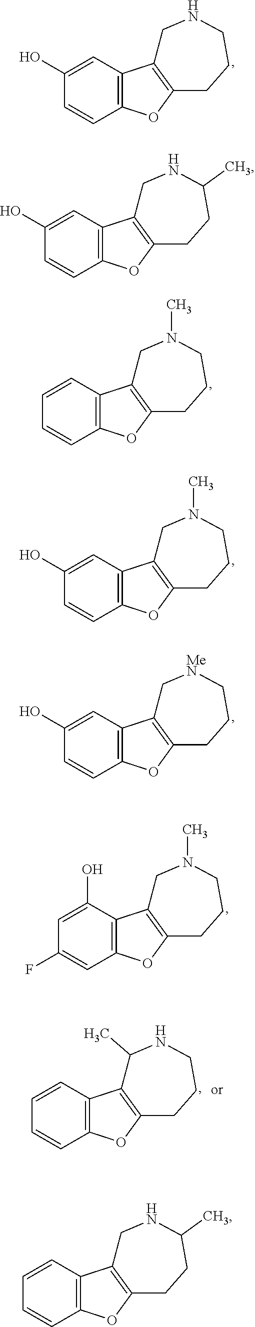

or a pharmaceutically acceptable salt thereof.

In some embodiments, the compound wherein D is NR₁, E is CR₂R₃, and F is CR₆R₇.

In some embodiments, the compound wherein X₁ is C and X₂ is NR₁₄, or X₁ is C and X₂ is O, or X₁ is C and X₂ is S, or X₁ is N and X₂ is CR₁₅, or X₁ is N and X₂ is N.

In certain embodiments the compound has the structure below wherein the substituents are defined as in the paragraphs below:

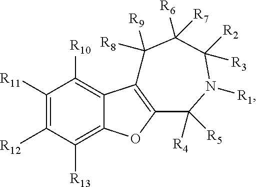

or a pharmaceutically acceptable salt thereof.

In some embodiments, the compound wherein R₁ is H or -(alkyl).

In some embodiments, the compound wherein R₁ is H, —CH₃ or —CH₂CH₅.

In some embodiments, the compound wherein R₂, R₃, R₄ and R₅ are each independently H, -(alkyl), -alkylcycloalkyl or -alkylaryl.

In some embodiments, the compound wherein R₂, R₃, R₄ and R₅ are each independently H, —CH₃, —CH₂CH₃, —CH₂CH₂CH₃ or —CH(CH₃)₂.

In some embodiments, the compound wherein R₂, R₃, R₄, R₅, R₆, R₇, R₈ and R₉ are each H.

In some embodiments, the compound wherein R₁₀, R₁₁, R₁₂ and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂ and R₁₃ are each independently H, —CH₃, OH, —OCH₃, —SCH₃, —CF₃ or F.

In some embodiments, the compound wherein R₁ is H or —CH₃.

In some embodiments, the compound having the structure:

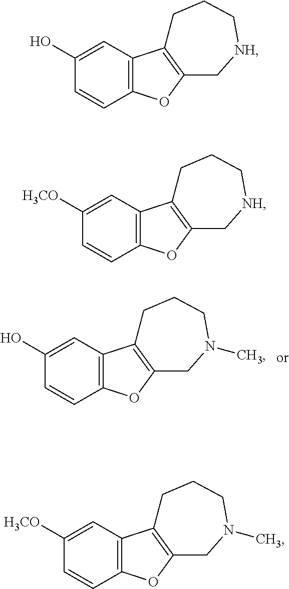

or a pharmaceutically acceptable salt thereof.

In some embodiments, the compound having the structure:

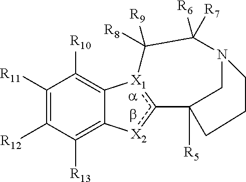

wherein
X₁ is C or N;
X₂ is O, S, N or NR₁₄,
   wherein R₁₄ is H, -(alkyl) or -cycloalkyl;
α and β represent a bond that is present or absent, and wherein either α or β is present,
   wherein when a is present, then X₁ is C and X₂ is O, S or NR₁₄, or
   when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and R₅, R₆, R₇, R₈, R₉, R₁₀, R₁₂, R₁₃ and R₁₄ are each H, then R₁₁ is other than H, F or —CH₃,

or a pharmaceutically acceptable salt or ester thereof.

In some embodiments, the compound having the structure:

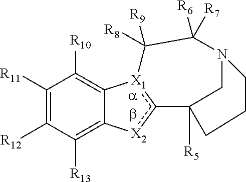

wherein
X₁ is C or N;
X₂ is O, S, N or NR₁₄,
   wherein R₁₄ is H, -(alkyl) or -cycloalkyl;
α and β represent a bond that is present or absent, and wherein either α or β is present,
   wherein when α is present, then X₁ is C and X₂ is O, S or NR₁₄, or
   when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON (alkyl)₂;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and R₅, R₆, R₇, R₈, R₉, R₁₀, R₁₂, R₁₃ and R₁₄ are each H, then Ru is other than H, F or —CH₃,

or a pharmaceutically acceptable salt or ester thereof.

In some embodiments, the compound wherein
X₁ is C or N;
X₂ is O, S or N;
α and β represent a bond that is present or absent, and wherein either α or β is present,
   wherein when α is present, then X₁ is C and X₂ is O or S, or
   when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON (alkyl)₂;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;

or a pharmaceutically acceptable salt or ester thereof.

In some embodiments, the compound wherein
R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN.

In some embodiments, the compound wherein
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂.

In some embodiments, the compound wherein

R₁₀ and R₁₁ together form a —O(CH₂)O— or R₁₁ and R₁₂ together form a —O(CH₂)O— or R₁₂ and R₁₃ together form a —O(CH₂)O—.

In certain embodiments the compound has the structure below wherein the substituents are defined as in the paragraphs above:

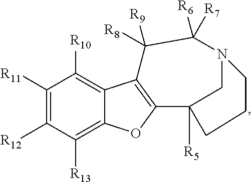

or a pharmaceutically acceptable salt or ester thereof.

In certain embodiments the compound has the structure below wherein the substituents are defined as in the paragraphs above:

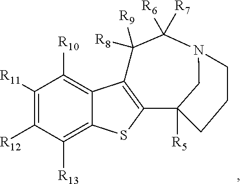

or a pharmaceutically acceptable salt or ester thereof.

In certain embodiments the compound has the structure below wherein the substituents are defined as in the paragraphs above:

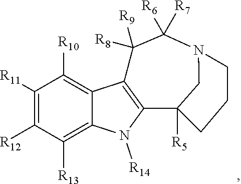

or a pharmaceutically acceptable salt or ester thereof.

In some embodiments, the compound wherein
R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN.

In some embodiments, the compound wherein
R₅ is H, -(alkyl), —OH, —O(alkyl), —OAc, —S(alkyl), —CO₂(alkyl), —CONH₂, —CONH(alkyl), —CON(alkyl)₂ or —CN.

In some embodiments, the compound wherein R₅, R₈ and R₉ are each independently H, -(alkyl), -alkylcycloalkyl, -alkylaryl, —O(alkyl), —S(alkyl), —OAc, —CO₂(alkyl), and R₆ and R₇ are each independently H, -(alkyl), -alkylcycloalkyl or -alkylaryl.

In some embodiments, the compound wherein R₅, R₈ and R₉ are each independently H, —CH₃, —CH₂CH₃, —CH₂CH₂CH₃, —CH(CH₃)₂ or —CO₂Me and R₆ and R₇ are each independently H, —CH₃, —CH₂CH₃, —CH₂CH₂CH₃, —CH(CH₃)₂ or —CO₂Me.

In some embodiments, the compound wherein R₅, R₆, R₇, R₈ and R₉ are each H.

In some embodiments, the compound wherein R₅, R₆ and R₇ are each H.

In some embodiments, the compound wherein R₅, R₈ and R₉ are each H.

In some embodiments, the compound wherein R₆ is —CH₃, and R₅, R₇, R₈ and R₉ are each H.

In some embodiments, the compound wherein R₁₀, R₁₁, R₁₂, and R₁₃ are each independently H, -(alkyl), OH, —O(alkyl), —S(alkyl), OAc, —CO₂(alkyl), —CF₃ or halogen.

In some embodiments, the compound wherein R₁₀, R₁₁, R₁₂ and R₁₃ are each independently H, —CH₃, OH, —OCH₃, —SCH₃, —CF₃ or F.

In some embodiments, the compound wherein R₁₀, R₁₁, R₁₂ and R₁₃ are each independently H, —CH₃, —CH₂CH₃, —CH(CH₃)₂, —OH, —OCH₃, —OCH₂CH₃, —SCH₃, —CF₃, F or Cl.

In some embodiments, the compound wherein R₁₀, R₁₁, R₁₂ and R₁₃ are each independently H, —CH₃, —CH₂CH₃, —CH(CH₃)₂, cyclopropyl, —OH, —OCH₃, —OCH₂CH₃, —SCH₃, —CF₃, F, Cl or NO₂.

In some embodiments, the compound wherein R₁₀ and R₁₁ together form a —O(CH₂)O—, R₁₁ and R₁₂ together form a —O(CH₂)O— or R₁₂ and R₁₃ together form a —O(CH₂)O—.

In some embodiments, the compound wherein R₁₀, R₁₁, R₁₂ and R₁₃ are each H.

In some embodiments, the compound wherein R₁₀, R₁₂ and R₁₃ are H, and R₁₁ is OH.

In some embodiments, the compound wherein R₁₀, R₁₂ and R₁₃ are H, and R₁₁ is —O (alkyl).

In some embodiments, the compound wherein R₁₀, R₁₂ and R₁₃ are H, and R₁₁ is —OCH₃.

In certain embodiments the compound has the structure below wherein the substituents are defined as in the paragraphs above:

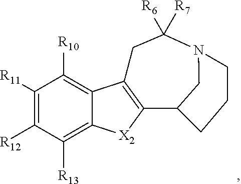

or a pharmaceutically acceptable salt or ester thereof.

In some embodiments, the compound wherein X₂ is O, R₆ is —CH₃, R₇ is H, and R₁₁ is —OH.

In some embodiments, the compound wherein X₂ is NR₁₄, R₆ is —CH₃, R₇ is H, and R₁₁ is —OH, wherein R₁₄ is H.

In certain embodiments the compound has the structure below wherein the substituents are defined as in the paragraphs above:

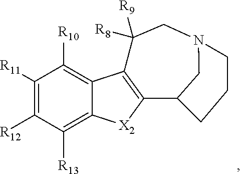

or a pharmaceutically acceptable salt or ester thereof.

In certain embodiments the compound has the structure below wherein the substituents are defined as in the paragraphs above:

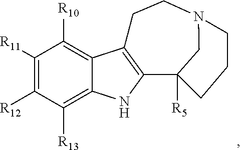

or a pharmaceutically acceptable salt or ester thereof.

In some embodiments, the compound wherein R₅ is H, -(alkyl), —OH, —O(alkyl), —OAc, —S(alkyl), —CO₂(alkyl), —CONH₂, —CONH(alkyl), —CON(alkyl)₂ or —CN.

In some embodiments, the compound wherein R₅ is —CO₂Me, and R₁₀, R₁₁, R₁₂ and R₁₃ are each H.

In some embodiments, the compound wherein R₅ is —CO₂Me, R₁₁ is OH, and R₁₀, R₁₂ and R₁₃ are each H.

In some embodiments, the compound wherein R₅ is —CO₂Me, R₁₁ is —OCH₃, and R₁₀, R₁₂ and R₁₃ are each H.

In some embodiments, the compound having the structure:

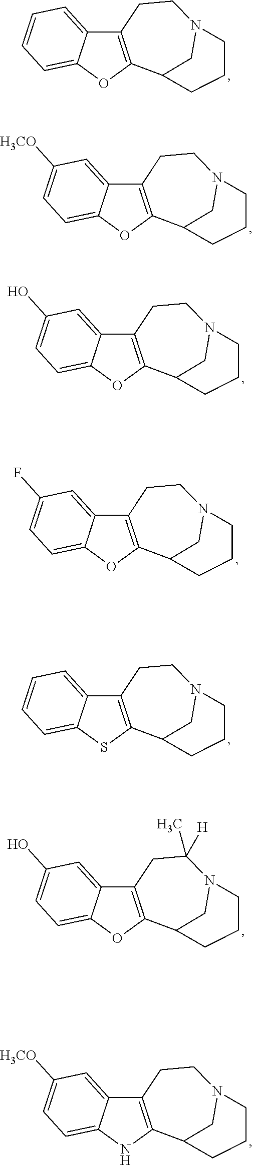

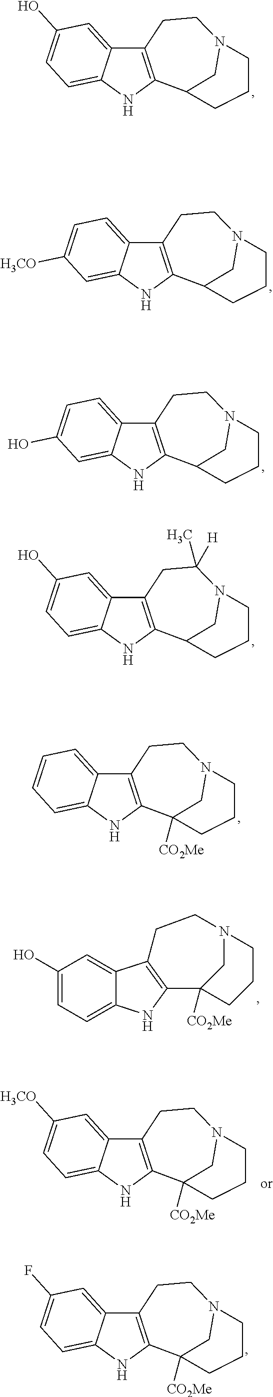

or a pharmaceutically acceptable salt thereof.

In some embodiments, the compound having the structure:

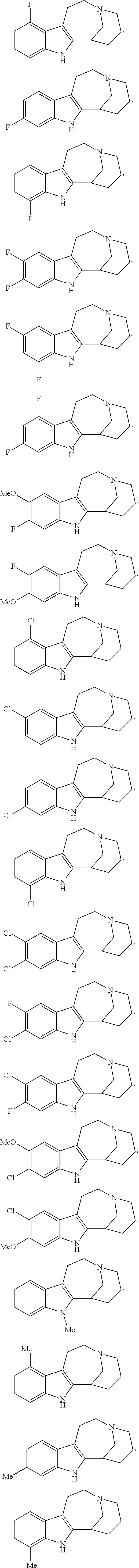

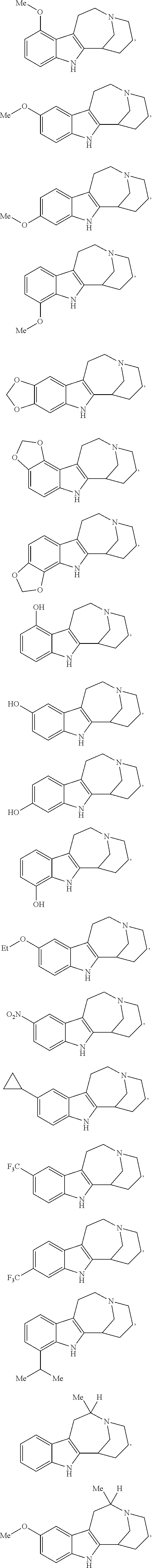

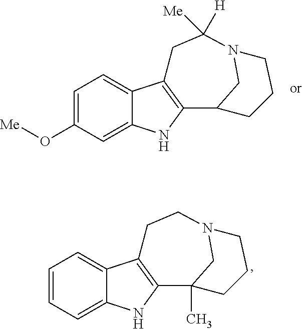 [SMILES: COc1ccc2c3c([nH]c2c1)C1CCCN(C1)C(C)C3 ; CC12CCCN(CCc3c1[nH]c1ccccc31)C2]

or a pharmaceutically acceptable salt thereof.

The present invention provides a pharmaceutical composition comprising the compound of the present invention and a pharmaceutically acceptable carrier.

The present invention provides a pharmaceutical composition comprising the compound having the structure:

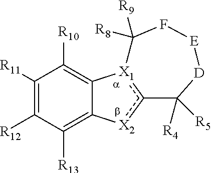

wherein
D, E and F are each independently NR₁, CR₂R₃ or CR₆R₇, wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅; R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

and a pharmaceutically acceptable carrier.

The present invention provides a pharmaceutical composition comprising the compound having the structure:

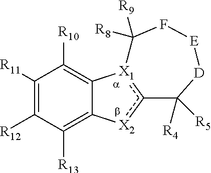

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON (alkyl)₂,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,

and a pharmaceutically acceptable carrier.

The present invention provides a method of activating 5HT2A, 5HT2C, or both 5HT2A and 5HT2C receptors comprising contacting the 5HT2A and 5HT2C receptors with the compound of the present invention.

The present invention provides a method of inhibiting SERT receptor comprising contacting the SERT receptor with the compound of the present invention.

The present invention provides a method of activating kappa-opioid receptor comprising contacting the kappa-opioid receptor with the compound of the present invention.

The present invention provides a method of inhibiting nicotinic acetylcholine receptor comprising contacting the nicotinic acetylcholine receptor with the compound of the present invention.

In an embodiment of any of the above methods, the nicotinic acetylcholine receptor is α3β4.

The present invention provides a method of treating a subject afflicted with substance use disorder comprising administering to the subject the compound of the present invention, or the composition of the present invention comprising an effective amount of the compound, so as to thereby treat the subject afflicted with the substance use disorder.

In some embodiments, wherein the substance use disorder is opioid use disorder, alcohol use disorder or stimulant use disorder.

The present invention provides a method of treating a subject afflicted with opioid withdrawal symptoms comprising administering to the subject the compound of the present invention, or the composition of the present invention comprising an effective amount of the compound, so as to thereby treat the subject afflicted with the opioid withdrawal symptoms.

The present invention provides a method of altering the psychological state of a subject comprising administering to the subject the compound of the present invention, or the composition of the present invention comprising an effective amount of the compound, so as to thereby alter the psychological state of the subject.

The present invention provides a method of enhancing the effect of psychotherapy in a subject comprising administering to the subject the compound of the present invention, or the composition of the present invention comprising an effective amount of the compound, so as to thereby enhance the effect of the psychotherapy in the subject.

The present invention provides a method of treating a subject afflicted with a depressive disorder, a mood disorder, an anxiety disorder, Parkinson's disease, or traumatic brain injury comprising administering to the subject the compound of the present invention, or the composition of the present invention comprising an effective amount of the compound, so as to thereby treat the subject afflicted with the depressive disorder, the mood disorder, the anxiety disorder, Parkinson's disease or the traumatic brain injury.

The present invention provides a method of treating a subject afflicted with a headache or a migraine comprising administering to the subject the compound of the present invention, or the composition of the present invention comprising an effective amount of the compound, so as to thereby treat the subject afflicted with the headache or the migraine.

The present invention also provides a method of treating a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy, comprising administering to the subject an effective amount of a compound having the structure:

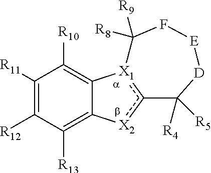

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,

or a pharmaceutically acceptable salt thereof, so as to thereby treat a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy.

The present invention also provides a method of treating a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy, comprising administering to the subject an effective amount of a compound having the structure:

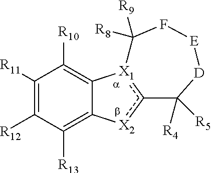

wherein
D, E and F are each independently NR₁, CR₂R₃ or CR₆R₇,
   wherein one of D, E and F is NR₁ and the remaining two are CR₂R₃ or CR₆R₇,
   wherein R₁ is H or -(alkyl), and
   wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
   wherein R₁₄ is H, -(alkyl) or -cycloalkyl,
   wherein R₁₅ is H, -(alkyl) or -cycloalkyl, and
   wherein X₂ is other than N when X₁ is N;
α and β represent a bond that is present or absent, and wherein either α or β is present,
   wherein when α is present, then X₁ is C and X₂ is O, S or NR₁₄, or
   when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
   wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
   wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,

or a pharmaceutically acceptable salt thereof, so as to thereby treat a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy.

In some embodiments of any of the above methods, comprising activating 5HT2A, 5HT2C, or both 5HT2A and 5HT2C receptors.

In some embodiments of any of the above methods, comprising inhibiting SERT receptor.

In some embodiments of any of the above methods, comprising activating kappa-opioid receptor.

In some embodiments of any of the above methods, comprising inhibiting nicotinic acetylcholine receptor.

In some embodiments of any of the above methods, wherein the nicotinic acetylcholine receptor is α3β4.

In some embodiments of any of the above methods, wherein the substance use disorder is opioid use disorder, alcohol use disorder or stimulant use disorder.

In some embodiments of any of the above methods, wherein the compound has the structure:

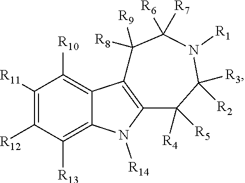

or a pharmaceutically acceptable salt thereof.

In some embodiments of any of the above methods, wherein the compound has the structure:

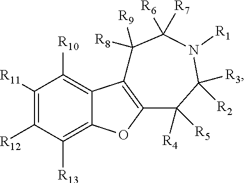

or a pharmaceutically acceptable salt thereof.

In some embodiments of any of the above methods, wherein the compound has the structure:

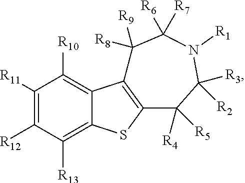

or a pharmaceutically acceptable salt thereof.

In some embodiments of any of the above methods, wherein the compound has the structure:

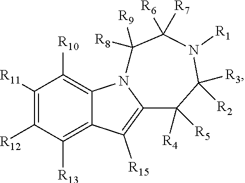

or a pharmaceutically acceptable salt thereof.

In some embodiments of any of the above methods, wherein the compound has the structure:

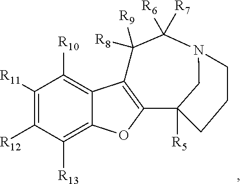

or a pharmaceutically acceptable salt or ester thereof.

In some embodiments of any of the above methods, wherein the compound has the structure:

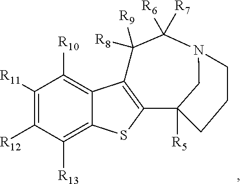

or a pharmaceutically acceptable salt or ester thereof.

In some embodiments of any of the above methods, wherein the compound has the structures

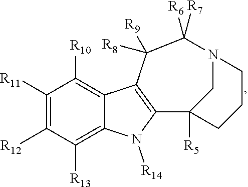

or a pharmaceutically acceptable salt or ester thereof.

In some embodiments of any of the above methods, wherein comprising treating a subject afflicted with substance use disorder.

In some embodiments of any of the above methods, wherein the substance use disorder is opioid use disorder, alcohol use disorder or stimulant use disorder.

In some embodiments of any of the above methods, wherein comprising treating a subject afflicted with opioid withdrawal symptoms.

In some embodiments of any of the above methods, wherein comprising altering the psychological state of a subject.

In some embodiments of any of the above methods, wherein comprising enhancing the effect of psychotherapy in a subject.

In some embodiments of any of the above methods, wherein comprising treating a subject afflicted with a depressive disorder, a mood disorder, an anxiety disorder, Parkinson's disease, or traumatic brain injury.

In some embodiments of any of the above methods, wherein comprising treating a subject afflicted with a headache or a migraine.

The present invention provides a composition which comprises a carrier and a compound having the structure of the present invention or a pharmaceutically acceptable salt of the compound.

In some embodiments, the composition further comprising a carrier.

In some embodiments, wherein the carrier is a pharmaceutically acceptable carrier.

The present invention provides a pharmaceutical composition comprising the compound of the present invention and a pharmaceutically acceptable carrier.

In some embodiments, the composition further comprising a mu-opioid receptor agonist.

In some embodiments, the composition further comprising an opioid or opiate.

In some embodiments, wherein the opioid or opiate is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepin-11-yl)amino)heptanoic acid, 7-((3-iodo-6-methyl-5,5-dioxido-6,11-dihydrodibenzo[c,f][1,2] thiazepin-11-yl)amino)heptanoic acid, 5-((3-bromo-6-methyl-5,5-dioxido-6,11-dihydrodibenzo[c,f] [1,2]thiazepin-11-yl)amino)pentanoic acid or 5-((3-iodo-6-methyl-5,5-dioxido-6,11-dihydrodibenzo[c,f][1,2]thiazepin-11-yl)amino)pentanoic acid.

The present invention provides a method of altering the psychological state of a subject comprising administering to the subject the compound of the present invention, or the composition of the present invention comprising an effective amount of the compound, so as to thereby alter the psychological state of the subject.

The present invention provides a method of enhancing the effect of psychotherapy in a subject comprising administering to the subject the compound of the present invention, or the composition of the present invention comprising an effective amount of the compound, so as to thereby enhance the effect of the psychotherapy in the subject.

The present invention provides a method of inducing wakefulness or decreasing sleepiness in a subject comprising administering to the subject the compound of the present invention, or the composition of the present invention comprising an effective amount of the compound, so as to thereby induce wakefulness or decrease sleepiness in the subject.

The present invention provides a method of decreasing the duration of REM sleep in a subject comprising administering to the subject the composition of the present invention comprising an effective amount of the compound so as to thereby decrease the duration of REM sleep in the subject.

The present invention provides a method of increasing energetic feelings in a subject comprising administering to the subject the composition of the present invention comprising an effective amount of the compound so as to thereby increase the energetic feelings in the subject.

The present invention provides a method of inducing a stimulating effect in a subject comprising administering to the subject the compound of the present invention, or the composition of the present invention comprising an effective amount of the compound, so as to thereby induce the stimulating effect in the subject.

In some embodiments, the stimulating effect is a central stimulating effect.

In some embodiments, the stimulating effect is induced substantially free of undesired side-effects in the subject.

In some embodiments, the stimulating effect is induced without inducing an addictive effect in the subject to the compound.

In some embodiments, a use of the composition of the present invention comprising an effective amount of the compound as a stimulant.

The present invention provides a method of treating a subject afflicted with substance use disorder comprising administering to the subject the compound of the present invention, or the composition of the present invention comprising an effective amount of the compound, so as to thereby treat the subject afflicted with the substance use disorder.

In some embodiments, wherein the substance use disorder is opioid use disorder, alcohol use disorder or stimulant use disorder.

The present invention provides a method of treating a subject afflicted with opioid withdrawal symptoms comprising administering to the subject the compound of the present invention, or the composition of the present invention comprising an effective amount of the compound, so as to thereby treat the subject afflicted with the opioid withdrawal symptoms.

The present invention provides a method of treating a subject afflicted with a depressive disorder, a mood disorder, an anxiety disorder, Parkinson's disease, or traumatic brain injury comprising administering to the subject the compound of the present invention, or the composition of the present invention comprising an effective amount of the compound, so as to thereby treat the subject afflicted with the depressive disorder, the mood disorder, the anxiety disorder, Parkinson's disease or the traumatic brain injury.

The present invention provides a method of treating a subject afflicted with a headache or a migraine comprising administering to the subject the compound of the present invention, or the composition of the present invention comprising an effective amount of the compound, so as to thereby treat the subject afflicted with the headache or the migraine.

The present invention provides a method of treating a subject afflicted with pain comprising administering to the subject the composition of the present invention comprising an effective amount of the compound and the opioid or opiate so as to thereby treat the subject afflicted with pain.

In some embodiments, wherein an effective amount of 10-1500 mg of the compound is administered to the subject.

In some embodiments of any of the above composition, the composition further comprising a carrier.

In some embodiments of any of the above composition, the composition wherein the carrier is a pharmaceutically acceptable carrier.

In some embodiments of any of the above compositions, the composition further comprising a mu-opioid receptor agonist.

In some embodiments of any of the above compositions, the composition further comprising an opioid or opiate.

In some embodiments of any of the above compositions, the composition further comprising morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepin-11-yl)amino)heptanoic acid, 7-((3-iodo-6-methyl-5,5-dioxido-6,11-dihydrodibenzo[c,f][1,2] thiazepin-11-yl)amino)heptanoic acid, 5-((3-bromo-6-methyl-5,5-dioxido-6,11-dihydrodibenzo[c,f][1,2]thiazepin-11-yl)amino)pentanoic acid or 5-((3-iodo-6-methyl-5,5-dioxido-6,11-dihydrodibenzo[c,f][1,2]thiazepin-11-yl)amino)pentanoic acid.

In some embodiments of any of the above compositions, the composition further comprising any of the compounds disclosed in PCT International Publication Nos. WO 2015/138791, WO 2017/049158, WO 2018/170275 or WO 2020/037136, the contents of each of which are hereby incorporated by reference.

In some embodiments, a method of altering the psychological state of a subject comprising administering to the subject the composition of the present invention comprising an effective amount of the compound so as to thereby alter the psychological state of the subject.

In some embodiments, a method of enhancing the effect of psychotherapy comprising administering to the subject the composition of the present invention comprising an effective amount of the compound so as to thereby enhance the effect of the psychotherapy.

In some embodiments, a method of treating a subject afflicted with a depressive disorder, a mood disorder or an anxiety disorder, comprising administering to the subject the composition of the present invention comprising an effective amount of the compound so as to thereby treat the subject afflicted with the depressive disorder, the mood disorder or the anxiety disorder.

In some embodiments, the depressive disorder, the mood disorder, or the anxiety disorder.

In some embodiments, a method of reducing opioid cravings in a subject afflicted with an opioid use disorder comprising administering to the subject the composition of the present invention comprising an effective amount of the compound so as to reduce the subject's opioid cravings.

In some embodiments, a method of treating a subject afflicted with a substance use disorder comprising administering to the subject the composition of the present invention comprising an effective amount of the compound so as to treat the subject afflicted with the substance use disorder.

In some embodiments, wherein the substance use disorder is opioid use disorder, alcohol use disorder or stimulant use disorder.

In some embodiments, wherein the substance use disorder is opioid use disorder, alcohol use disorder, stimulant use disorder or polydrug use disorder.

In some embodiments, wherein the stimulant use disorder is nicotine use disorder.

In some embodiments, a method of treating a subject afflicted with opioid withdrawal symptoms comprising administering to the subject the composition of the present invention comprising an effective amount of the compound so as to treat the subject afflicted with the opioid withdrawal symptoms.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with alcohol withdrawal symptoms or stimulant withdrawal symptoms comprising administering to the subject the composition of the present invention comprising an effective amount of the compound so as to treat the subject afflicted with the opioid withdrawal symptoms.

In some embodiments, a method of treating a subject afflicted with traumatic brain injury (TBI) comprising administering to the subject the composition of the present invention comprising an effective amount of the compound so as to treat the subject afflicted with the traumatic brain injury (TBI).

In some embodiments, a method of treating a subject afflicted with Parkinson's disease comprising administering to the subject the composition of the present invention comprising an effective amount of the compound so as to treat the subject afflicted with the Parkinson's disease.

In some embodiments, a method of treating a subject afflicted with a headache or a migraine comprising administering to the subject the composition of the present invention comprising an effective amount of the compound so as to treat the subject afflicted with a headache or a migraine.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with pain comprising administering to the subject an effective amount of an opioid or opiate and the composition of the present invention comprising an effective amount of the compound so as to treat the subject afflicted with pain.

In some embodiments, a method of treating a subject afflicted with pain comprising administering to the subject an effective amount of morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepin-11-yl)amino)heptanoic acid, 7-((3-iodo-6-methyl-5,5-dioxido-6,11-dihydrodibenzo[c,f] [1,2] thiazepin-11-yl)amino)heptanoic acid, 5-((3-bromo-6-methyl-5,5-dioxido-6,11-dihydrodibenzo[c,f][1,2]thiazepin-11-yl)amino)pentanoic acid or 5-((3-iodo-6-methyl-5,5-dioxido-6,11-dihydrodibenzo[c,f] [1,2]thiazepin-11-yl)amino)pentanoic acid and the composition of the present invention comprising an effective amount of the compound so as to treat the subject afflicted with pain.

In some embodiments, a method of treating a subject afflicted with opioid use disorder comprising administering to the subject an effective amount of an opioid or opiate and the composition of the present invention comprising an effective amount of the compound so as to treat the subject afflicted with the opioid use disorder.

In some embodiments, a method of treating a subject afflicted with opioid use disorder comprising administering to the subject an effective amount of morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepin-11-yl)amino)heptanoic acid, 7-((3-iodo-6-methyl-5,5-dioxido-6,11-dihydrodibenzo[c,f] [1,2] thiazepin-11-yl)amino)heptanoic acid, 5-((3-bromo-6-methyl-5,5-dioxido-6,11-dihydrodibenzo[c,f][1,2]thiazepin-11-yl)amino)pentanoic acid or 5-((3-iodo-6-methyl-5,5-dioxido-6,11-dihydrodibenzo[c,f] [1,2]thiazepin-11-yl)amino)pentanoic acid and the composition of the present invention comprising an effective amount of the compound so as to treat the subject afflicted with the opioid use disorder.

In some embodiments, a method of treating a subject afflicted with opioid use disorder or opioid withdrawal symptoms comprising administering to the subject an effective amount of naloxone or methylnaltrexone and the composition of the present invention comprising an effective amount of the compound so as to thereby treat the subject afflicted with the opioid use disorder or opioid withdrawal symptoms.

In some embodiments, a method of treating a subject afflicted with substance use disorder or opioid withdrawal symptoms comprising administering to the subject an effective amount of Suboxone or Naltrexone and the composition of the present invention comprising an effective amount of the compound so as to thereby treat the subject afflicted with the opioid use disorder or opioid withdrawal symptoms.

In some embodiments, a package comprising:
a) a first pharmaceutical composition comprising an amount of an opioid or opiate and a pharmaceutically acceptable carrier;
b) a second pharmaceutical composition comprising the compound of the present invention and a pharmaceutically acceptable carrier; and
c) instructions for use of the first and second pharmaceutical compositions together to treat a subject afflicted with pain, a depressive disorder, a mood disorder, an anxiety disorder, a substance use disorder, opioid withdrawal symptoms, traumatic brain injury, or Parkinson's disease.

In some embodiments, a therapeutic package for dispensing to, or for use in dispensing to, a subject afflicted pain, a depressive disorder, a mood disorder, an anxiety disorder, a substance use disorder, opioid withdrawal symptoms, traumatic brain injury or Parkinson's disease, which comprises:
a) one or more unit doses, each such unit dose comprising:
   (i) a pharmaceutical composition comprising the compound of the present invention; and
   (ii) an amount of an opioid or opiate, wherein the respective amounts of said composition and said opioid or opiate in said unit dose are effective, upon concomitant administration to said subject, to treat the subject, and
(b) a finished pharmaceutical container therefor, said container containing said unit dose or unit doses, said container further containing or comprising labeling directing the use of said package in the treatment of said subject.

The therapeutic package of the above embodiment, wherein the respective amounts of said composition and opioid or opiate in said unit dose when taken together is more effective to treat the subject than when compared to the administration of said composition in the absence of said opioid or opiate or the administration of said opioid or opiate in the absence of said composition.

A pharmaceutical composition in unit dosage form, useful in treating a subject afflicted with pain, a depressive disorder, a mood disorder, an anxiety disorder, a substance use disorder, opioid withdrawal symptoms, traumatic brain injury or Parkinson's disease, which comprises:
(i) a composition comprising the compound of the present invention; and
(ii) an amount of an opioid or opiate,
wherein the respective amounts of said composition and said opioid or opiate in said composition are effective, upon concomitant administration to said subject of one or more of said unit dosage forms of said composition, to treat the subject.

The pharmaceutical composition of the above embodiment, wherein the respective amounts of said compound and said opioid or opiate in said unit dose when taken together is more effective to treat the subject than when compared to the administration of said composition in the absence of said opioid or opiate or the administration of said opioid or opiate in the absence of said composition.

In some embodiments of the present method, package, use or pharmaceutical composition, the compound has the structure:

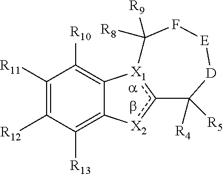

In some embodiments, a pharmaceutically acceptable salt of any of the above compounds of the present invention.

In some embodiments, a salt of the compound of the present invention is used in any of the above methods, uses, packages or compositions.

In some embodiments, a pharmaceutically acceptable salt of the compound of the present invention is used in any of the above methods, uses, packages or compositions.

Any of the above compounds may be used in any of the disclosed methods, uses, packages or pharmaceutical compositions.

Any of the compounds used in the disclosed methods, uses, packages or pharmaceutical compositions may be replaced with any other compound disclosed in the present invention.

Any of the above generic compounds may be used in any of the disclosed methods, uses, packages or compositions.

In some embodiments of any of the above methods, wherein the composition is orally administered to the subject.

In some embodiments of any of the above methods, wherein 10-30 mg of the compound is administered to the subject.

In some embodiments of any of the above methods, wherein 30-100 mg of the compound is administered to the subject.

In some embodiments of any of the above methods, wherein 100-300 mg of the compound is administered to the subject.

In some embodiments of any of the above methods, wherein 300-500 mg of the compound is administered to the subject.

In some embodiments of any of the above methods, wherein 500-800 mg of the compound is administered to the subject.

In some embodiments of any of the above methods, wherein 800-1100 mg of the compound is administered to the subject.

In some embodiments of any of the above methods, wherein 1200-1500 mg of the compound is administered to the subject.

In some embodiments, a method wherein any of the above recited doses of the compound, and an opioid are administered to a subject afflicted with a substance use disorder, opioid withdrawal symptoms, pain, a mood disorder, an anxiety disorder or opioid cravings so as to thereby treat the subject afflicted with the substance use disorder, opioid withdrawal symptoms, pain or the mood disorder or reduce opioid cravings in the subject.

In some embodiments of any of the above methods, wherein the opioid is morphine and 10-20 mg (oral) or 3-5 mg (parenteral) of the opioid is administered to the subject.

In some embodiments of any of the above methods, wherein the opioid is codeine and 30-60 mg (oral) of the opioid is administered to the subject.

In some embodiments of any of the above methods, wherein the opioid is oxycodone and 5-10 mg (oral) of the opioid is administered to the subject.

In some embodiments of any of the above methods, wherein the opioid is fentanyl and 40-60 μg (parenteral) of the opioid is administered to the subject.

In some embodiments of any of the above methods, wherein the opioid is butorphanol and 1-3 mg (parenteral) of the opioid is administered to the subject.

In some embodiments of any of the above methods, wherein the opioid is nalbuphine and 5-15 mg (parenteral) of the opioid is administered to the subject.

In some embodiments of any of the above methods, wherein mitragynine (15-100 mg-oral) or 3-deuteromitragynine (15-100 mg-oral) is administered to the subject.

In some embodiments of any of the above methods, wherein tianeptine (12.5-100 mg-oral) is administered to the subject.

In some embodiments of any of the above methods, wherein 7-((3-iodo-6-methyl-5,5-dioxido-6,11-dihydrodibenzo[c,f][1,2] thiazepin-11-yl)amino)heptanoic acid (1.5-10 mg-oral) is administered to the subject.

In some embodiments of any of the above methods, wherein 5-((3-iodo-6-methyl-5,5-dioxido-6,11-dihydrodibenzo[c,f][1,2]thiazepin-11-yl)amino)pentanoic acid (2-20 mg-oral) is administered to the subject.

In some embodiments of any of the above methods, wherein administration of the composition of the present invention comprising an effective amount of the compound lowers the effective amount of the opioid.

In some embodiments of any of the above methods, wherein administration of the composition of the present invention lowers the effective dosage amount of the opioid by 75% or more.

In some embodiments of the above method, wherein administration of the composition of the present invention lowers the effective dosage amount of the opioid by 50% or more.

In some embodiments of the above method, wherein administration of the composition of the present invention lowers the effective dosage amount of the opioid by 25% or more.

In some embodiments of any of the above methods, wherein 0.4 mg/kg-30 mg/kg of the compound of the present invention is administered to the subject.

In some embodiments of any of the above methods, wherein 0.3-1.5 mg/kg of the opioid or opiate is administered to the subject.

In some embodiments of any of the above methods, wherein the subject is a human.

In some embodiments of any of the above methods, the composition is clinic administered or physician administered to the subject.

In some embodiments of any of the above methods, the composition is clinic self-administered by the subject.

In some embodiments, the method wherein the subject is afflicted with a depressive disorder, a mood disorder, or an anxiety disorder.

In some embodiments, the anxiety disorder includes, but is not limited to, anxiety, generalized anxiety disorder (GAD), panic disorder, social phobia, social anxiety disorder, acute stress disorder, obsessive-compulsive disorder (OCD), or post-traumatic stress disorder (PTSD).

In some embodiments, the depressive disorder includes, but is not limited to, depression, major depression, dysthymia, cyclothymia, postpartum depression, seasonal affective disorder, atypical depression, psychotic depression, bipolar disorder, premenstrual dysphoric disorder, situational depression or adjustment disorder with depressed mood. Depressive disorders can also include other mood disorders and is not limited to the above list.

Preclinical evidence (rodents) also shows that ibogaine/noribogaine enhances morphine's analgesic effect (Sharma, S. S. et al. 1998) or reverses analgesic tolerance to morphine (Bhargava, H. N. et al. 1997).

In some embodiments, the method wherein the subject is afflicted with pain. Reports of stimulant effects of Tabernanthe iboga date back to late 1890's and early 1900's in the descriptions of ritual and medicinal use by the native inhabitants in Africa. Ibogaine was recommended in France to treat “asthenia” (dose range of 10-30 mg per day). In the period of 1939-1970, ibogaine was commercially available in France as “Lambarène”, a “neuromuscular stimulant” (8 mg pills) recommended for fatigue, depression, and recovery from infectious diseases (Alper, K. R. 2001). In one clinical study, subjects took visual analog scale tests (VAS, 0-100) related to sleepiness, energetic feelings, and the side effects such as nausea, anxiety versus calmness. Subjects reported that ibogaine decreased sleepiness and increased energetic feeling over the examined 24-hour period after one dose of 20 mg of ibogaine (Glue, P. et al. 2015). A stimulant effect was reported in cats (Schneider et. al 1957). In rats, ibogaine induced wakefulness and suppressed the REM sleep as shown via EEG (Gonzalez, J. et al 2018).

It has been shown in rats that ibogaine leads to a dramatic upregulation of BDNF (in addition to Glial cell line-Derived Neurotrophic Factor (GDNF)) which provides structural and functional restorative effects in subjects afflicted with TBI (Marton, S. et al. 2019). The efficacy of ibogaine has also been shown in cases of soldiers afflicted with TBI and PTSD (Thoricatha, W. 2020).

In some embodiments, the method wherein the subject is afflicted with traumatic brain injury (TBI).

It has been shown in rats that ibogaine induces expression of GDNF (He, D-Y. et al. 2005 and Marton, S. et al. 2019), a critical neurotrophic factor that maintains and restores the dopaminergic system (which degenerates in Parkinson's disease). Thus, ibogaine provides structural and functional restorative effects in subjects afflicted with Parkinson's disease. GDNF itself has been shown to exert desired effects in Parkinson's rodent and monkey models (Gash, D. M. et al. 1996).

In some embodiments, the method wherein the subject is afflicted with Parkinson's disease.

It has been shown in humans that ibogaine is useful in treating opioid and stimulant use disorders (Alper, K. R. et al. 1999; Mash, D. C. et al. 2018; Schenberg, E. E. et al. 2014) or in maintenance therapy (opioid use disorder) in combination with an opioid to lower effective opioid doses (Kroupa, P. K. & Wells, H. 2005).

In some embodiments, wherein the substance use disorder is an opioid use disorder, alcohol use disorder or stimulant use disorder.

Opioid use disorder (OUD) involves, but is not limited to, misuse of opioid medication or use of illicitly obtained opioids. The Diagnostic and Statistical Manual of Mental Disorders, 5th Edition (American Psychiatric Association: Diagnostic and Statistical Manual of Mental Disorders: Diagnostic and Statistical Manual of Mental Disorders, Fifth Edition. Arlington, VA: American Psychiatric Association, 2013), which is hereby incorporated by reference, describes opioid use disorder as a problematic pattern of opioid use leading to problems or distress, with at least two of the following occurring within a 12-month period:
Taking larger amounts or taking drugs over a longer period than intended.
Persistent desire or unsuccessful efforts to cut down or control opioid use.
Spending a great deal of time obtaining or using the opioid or recovering from its effects.
Craving, or a strong desire or urge to use opioids.
Problems fulfilling obligations at work, school, or home.
Continued opioid use despite having recurring social or interpersonal problems.
Giving up or reducing activities because of opioid use.
Using opioids in physically hazardous situations.
Continued opioid use despite ongoing physical or psychological problem likely to have been caused or worsened by opioids.
Tolerance (i.e., need for increased amounts or diminished effect with continued use of the same amount).
Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5th Edition describes alcohol use disorder as a problematic pattern of alcohol use leading to problems or distress, with at least two of the following occurring within a 12-month period:
Being unable to limit the amount of alcohol you drink.
Wanting to cut down on how much you drink or making unsuccessful attempts to do so.
Spending a lot of time drinking, getting alcohol, or recovering from alcohol use.
Feeling a strong craving or urge to drink alcohol.
Failing to fulfill major obligations at work, school or home due to repeated alcohol use.
Continuing to drink alcohol even though you know it is causing physical, social, or interpersonal problems.
Giving up or reducing social and work activities and hobbies.
Using alcohol in situations where it is not safe, such as when driving or swimming.
Developing a tolerance to alcohol so you need more to feel its effect, or you have a reduced effect from the same amount.
Experiencing withdrawal symptoms—such as nausea, sweating and shaking—when you do not drink, or drinking to avoid these symptoms.

Stimulant use disorder involves, but is not limited to, a pattern of problematic use of amphetamine, methamphetamine, cocaine, or other stimulants except caffeine or nicotine leading to at least two of the following problems within a 12-month period:
Taking more stimulants than intended.
Unsuccessful in trying to cut down or control use of stimulants, despite wanting to do so.
Spending excessive amounts of time to activities surrounding stimulant use.
Urges and cravings for stimulants.
Failing in the obligations of home, school, or work.
Carrying on taking stimulants, even though it has led to relationship or social problems.
Giving up or reducing important recreational, social, or work-related activities because of using stimulants.
Using stimulants in a physically hazardous way.
Continuing to use stimulants even while knowing that it is causing or worsening a physical or psychological problem.
Tolerance to stimulants.
Withdrawal from stimulants if you do not take them.

Polydrug use disorder or polysubstance Use disorder involves, but is not limited to, dependence on multiple drugs or substances.

The term “MOR agonist” is intended to mean any compound or substance that activates the mu-opioid receptor (MOR). The agonist may be a partial, full, or super agonist.

In some embodiments, the compounds of the present invention may be safer and have fewer adverse effects compared to existing treatments.

In some embodiments, the compounds of the present invention may have better hERG profile/cardiac profile compared to ibogaine and noriboagine.

In some embodiments, the compounds of the present invention may be useful as tool compounds for studying the mechanism of ibogaine.

In an embodiment, the —(CH₂)ₘ— bridge connecting R₁ to R₄ is above the plane of the molecule. In another embodiment, the bridge connecting R₁ to R₄ is below the plane of the molecule.

In an embodiment m=2. In another embodiment m=3. In another embodiment m=4

A person skilled in the art may use the techniques disclosed herein to prepare deuterium analogs thereof.

Except where otherwise specified, the structure of a compound of this invention includes an asymmetric carbon atom, it is understood that the compound occurs as a racemate, racemic mixture, scalemic mixtures and isolated single enantiomers. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

Except where otherwise specified, the subject invention is intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notations of a carbon in structures throughout this application, when used without further notation, are intended to represent all isotopes of carbon, such as ¹²C, ¹³C, or ¹⁴C.

Furthermore, any compounds containing ¹³C or ¹⁴C may specifically have the structure of any of the compounds disclosed herein.

It will also be noted that any notations of a hydrogen (H) in structures throughout this application, when used without further notation, are intended to represent all isotopes of hydrogen, such as ¹H, ²H (D), or ³H (T) except where otherwise specified. Furthermore, any compounds containing ²H (D) or ³H (T) may specifically have the structure of any of the compounds disclosed herein except where otherwise specified.

Isotopically labeled compounds can generally be prepared by conventional techniques known to those skilled in the art using appropriate isotopically labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (²H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart.

As used herein, a hydrogen at a specific site in a compound is “deuterium-enriched” if the amount of deuterium at the specific site in the compound is more than the abundance of deuterium naturally occurring at that specific site in view of all of the molecules of the compound in a defined universe such as a composition or sample. Naturally occurring as used above refers to the abundance of deuterium which would be present at a relevant site in a compound if the compound was prepared without any affirmative step to enrich the abundance of deuterium. Thus, at a “deuterium-enriched” site in a compound, the abundance of deuterium at that site can range from more than 0.0156% to 100%. Examples of ways to obtain a deuterium-enriched site in a compound are exchanging hydrogen with deuterium or synthesizing the compound with deuterium-enriched starting materials.

In the compounds used in the method of the present invention, the substituents may be substituted or unsubstituted, unless specifically defined otherwise.

In the compounds used in the method of the present invention, alkyl, alkenyl, alkynyl, alkylaryl, cycloalkyl, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon-to-carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched, or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

The term “alkylcycloalkyl” refers to alkyl groups as described above wherein one or more bonds to hydrogen contained therein are replaced by a bond to a cycloalkyl group as described above. It is understood that an “alkylcycloalkyl” group is connected to a core molecule through a bond from the alkyl group and that the cycloalkyl group acts as a substituent on the alkyl group.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R—O—CO—R′ group.

The term “phenyl” is intended to mean an aromatic six membered ring containing six carbons.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound or composition of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect, refers to the relatively non-toxic, inorganic, and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease. Treating may also mean improving one or more symptoms of a disease.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin, and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention. Any of the disclosed generic or specific compounds may be applicable to any of the disclosed compositions, processes, or methods.

This invention will be better understood by reference to the Experimental Details which follow, but those skilled in the art will readily appreciate that the specific experiments detailed are only illustrative of the invention as described more fully in the claims, which follow thereafter.

### Experimental Details

General Considerations. Reagents and solvents were obtained from commercial sources and were used without further purification unless otherwise stated. Reactions were monitored by TLC using solvent mixtures appropriate to each reaction. Column chromatography was performed on silica gel (40-63 μm). For compounds containing a basic nitrogen, Et₃N was often used in the mobile phase to provide better resolution when using silica gel chromatography. In these cases, TLC plates were pre-soaked in the Et₃N-containing solvent and then allowed to dry briefly before use in analysis, such that an accurate representation of R_{f} was obtained. For preparative TLC, glass plates coated with a 1 mm silica layer were used. Nuclear magnetic resonance spectra were recorded on Bruker 400 or 500 MHz instruments, as indicated. Chemical shifts are reported as 5 values in ppm referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); dq (doublet of quartets); ddd (doublet of doublet of doublets); ddt (doublet of doublet of triplets); m (multiplet); br (broad). All carbon peaks are rounded to one decimal place unless such rounding would cause two close peaks to become identical; in these cases, two decimal places are retained. Low-resolution mass spectra were recorded on an Advion quadrupole instrument (ionization mode: APCI+ or ESI+) or on GC-MS (ionization mode: EI).

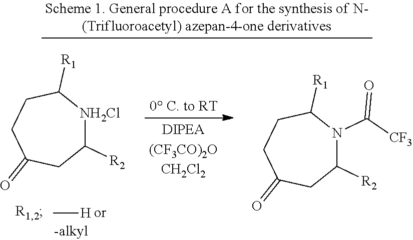

The corresponding azepan-4-one hydrochloride derivative (1 equivalent) was suspended in DCM (anhydrous, 0.5 M), DIPEA (2.5 equivalents) was added and the mixture was sonicated until all solid material dissolved. Reaction mixture was cooled using ice bath and trifluoroacetic anhydride (1.3 equivalents) was added dropwise over 5 minutes. Bright yellow mixture was further stirred at room temperature as indicated for each reaction and quenched by pouring into saturated aqueous NaHCO₃. Layers were separated and aqueous phase was further extracted with DCM (2×), and the combined organic extracts were dried over Na₂SO₄, filtered and concentrated. Crude material was purified as specified for each example.

### Example 1. N-(Trifluoroacetyl) azepan-4-one 1

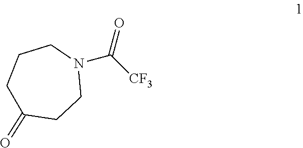 [SMILES: O=C1CCCN(C(=O)C(F)(F)F)CC1]

1 was prepared according to general procedure A (20 mmol scale, reaction time 4 h). Crude material was purified by column chromatography (silica gel, 30 to 40% ethyl acetate in hexanes). Product was obtained as a yellow oil that darkened to orange/red on prolonged storage (4.1 g, 90%). NMR spectra match previously reported characterization (Clement et al, 2018).

¹H NMR (500 MHz, CDCl₃) δ 3.86-3.70 (m, 4H), 2.77-2.67 (m, 4H), 1.96-1.87 (m, 2H). ¹⁹F NMR (471 MHz, CDCl₃) δ −67.9, −68.2.

### Example 2. N-(Trifluoroacetyl)-2-methylazepan-4-one 2

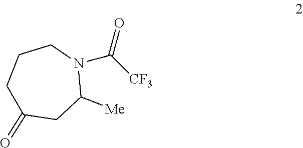 [SMILES: CC1CC(=O)CCCN1C(=O)C(F)(F)F]

2-Methylazepan-4-one hydrochloride was prepared according to published literature procedures (Lançois, D. F. A. et al 2016; Hartman 2015). Reaction was performed according to general procedure A (4.6 mmol scale, reaction time 19 h) using a higher excess of reagents (CF₃CO)₂O (2 equivalents) and DIPEA (4 equivalents). Crude material was purified by column chromatography (silica gel, 30 to 40% ethyl acetate in hexanes). Product was obtained as a yellow oil (0.46 g, 45%).

¹H NMR (500 MHz, CDCl₃) δ 4.80-4.67 (m, 0.5), 4.38 (dt, J=14.4, 4.0 Hz, 0.5H), 4.32 (dt, J=13.9, 6.8 Hz, 0.5H), 4.03-3.89 (m, 0.5H), 3.54-3.42 (m, 0.5H), 3.21-3.06 (m, 0.5H), 2.83-2.65 (m, 2H), 2.65-2.45 (m, 2H), 2.10-1.82 (m, 2H), 1.34 (d, J=6.5 Hz, 1H), 1.30 (d, J=6.8 Hz, 2H). ¹⁹F NMR δ −67.7, −68.0.

### Example 3. N-(Trifluoroacetyl)-7-methylazepan-4-one 3

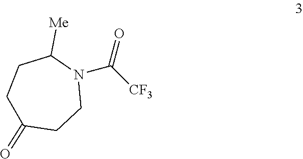 [SMILES: CC1CCC(=O)CCN1C(=O)C(F)(F)F]

7-Methylazepan-4-one hydrochloride was prepared according to published literature procedures (Lançois, D. F. A. et al 2016; Hartman 2015). Reaction was performed according to general procedure A (3.0 mmol scale, reaction time 15 h) using a higher excess of reagents (CF₃CO)₂O (1.5 equivalents) and DIPEA (3 equivalents). Crude material was purified by column chromatography (silica gel, gradient of 1:4, 1:3 to 1:2 ethyl acetate in hexanes). Product was obtained as an off-white solid (0.11 g, 17%).

¹H NMR (500 MHz, CDCl₃) δ 4.74 (dp, J=12.2, 6.2, 5.6 Hz, 0.33H), 4.28 (h, J=6.5 Hz, 0.66H), 4.13-4.03 (m, 0.66H), 3.84-3.75 (m, 0.33H), 3.55-3.44 (m, 0.33H), 3.29-3.17 (m, 0.66H), 2.84-2.74 (m, 0.66H), 2.74-2.57 (m, 1.66H), 2.51 (dtd, J=17.2, 3.0, 1.3 Hz, 0.66H), 2.47-2.33 (m, 1H), 2.13-1.97 (m, 1H), 1.87-1.70 (m, 1H), 1.32 (d, J=6.5 Hz, 2H), 1.21 (d, J=6.6 Hz, 1H). ¹⁹F NMR (471 MHz, CDCl3) δ −67.6, −68.4.

### Example 4. O-(4-bromophenyl) hydroxylamine hydrochloride 4

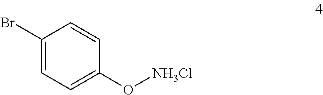

Compound 4 was prepared by a modification of a published procedure (Matsumura, Y. & Oyama, T. 2017). To a solution of 4-bromophenol (15 g, 87 mmol) in 2-propanol (13 mL), toluene (22 mL) and water (2.2 mL) was added potassium hydroxide (4.7 g, 87 mmol) and the mixture was heated to 50° C. A solution of hydroxylamine-O-sulfonic acid (4.95 g, 44 mmol) in water (13 mL) was added dropwise over 40 minutes to the reaction mixture and reaction was continued at 80° C. for 2 hours. The reaction mixture was cooled to room temperature, 10% aqueous sodium hydroxide solution was added, and the mixture was extracted twice with diethyl ether. The organic layer was washed with brine, dried over anhydrous sodium sulfate, filtered and concentrated. The resulting crude product was purified by column chromatography (gradient of 0 to 12% of ethyl acetate in hexanes+2% Et₃N). Isolated free base was dissolved in methanol, converted to a hydrochloride salt by addition of concentrated aqueous HCl (0.45 mL) and evaporated in vacuum. Compound 4 was isolated as a light brown crystalline powder (0.96 g, 10% yield). ¹H NMR (500 MHz, D₂O) δ 7.59 (m, J=7.9, 3.7 Hz, 2H), 7.17-7.00 (m, 2H).

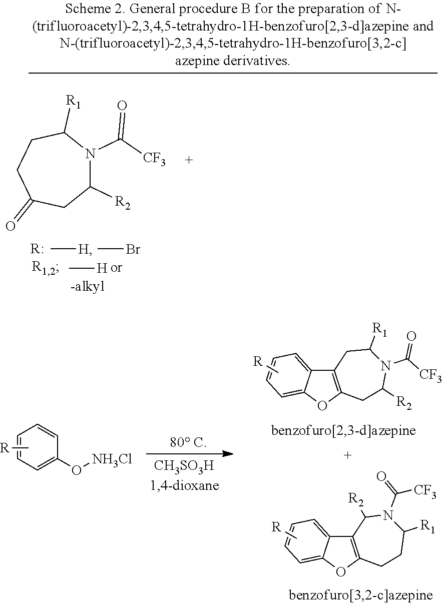

Corresponding N-(trifluoroacetyl)azepan-4-one (1 equivalent) and O-phenylhydroxylamine hydrochloride derivative were combined in 1,4-dioxane (anhydrous, 0.5 M) and warmed to 80° C. After 5 minutes at 80° C., methanesulfonic acid (2 equivalents) was added and reaction mixture was stirred at 80° C. for 5 hours. After cooling to RT reaction was quenched using saturated aqueous NaHCO₃ solution. The resulting mixture was extracted with diethyl ether (3×), combined organic extracts were dried over Na₂SO₄, filtered and concentrated. Crude material was purified as specified for each example.

### Example 5. N-(trifluoroacetyl)2,3,4,5-tetrahydro-1H-benzofuro[2,3-d]azepine 5 and N-(trifluoroacetyl)2,3,4,5-tetrahydro-1H-benzofuro[3,2-c]azepine 6

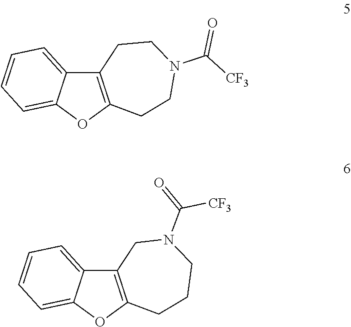 [SMILES: O=C(N1CCc2oc3ccccc3c2CC1)C(F)(F)F ; O=C(N1CCCc2oc3ccccc3c2C1)C(F)(F)F]

Compounds were synthesized according to general procedure B (2.0 mmol scale). Crude mixture of products was separated using column chromatography (gradient of 10, 15 to 20% diethyl ether in hexanes, crude material dry loaded on celite). Due to partial separation mixed fractions were combined and purified by preparative TLC (10% ethyl acetate in hexanes). Compound 5 was isolated as a pale-yellow oil (170 mg, 30%), compound 6 as a pale yellow solid (199 mg, 35%).

Compound 5

¹H NMR (400 MHz, CDCl₃) δ 7.45-7.37 (m, 2H), 7.29-7.19 (m, 2H), 4.01-3.85 (m, 4H), 3.24-3.13 (m, 2H), 3.01-2.92 (m, 2H). ¹⁹F NMR (471 MHz, CDCl₃) δ −68.0, −68.1.

Compound 6

¹H NMR (400 MHz, CDCl₃) δ 7.53 (dd, J=5.9, 3.2 Hz, 0.4H), 7.44-7.36 (m, 1.6H), 7.29-7.21 (m, 2H), 4.82 (s, 0.8H), 4.73 (s, 1.2H), 3.95-3.86 (m, 2H), 3.13-3.02 (m, 2H), 2.19-2.07 (m, 2H). ¹⁹F NMR (471 MHz, CDCl₃) δ −67.72, −67.75.

### Example 6. N-(trifluoroacetyl)2-methyl-2,3,4,5-tetrahydro-1H-benzofuro[2,3-d]azepine 7 and N-(trifluoroacetyl)3-methyl-2,3,4,5-tetrahydro-1H-benzofuro[3,2-c]azepine 8

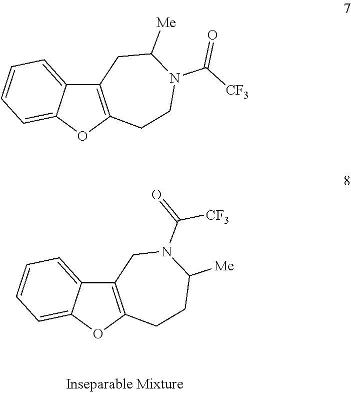 [SMILES: CC1Cc2c(oc3ccccc23)CCN1C(=O)C(F)(F)F ; CC1CCc2oc3ccccc3c2CN1C(=O)C(F)(F)F]

Compounds were synthesized according to general procedure B (0.5 mmol scale). Crude mixture of products was purified using column chromatography (5% ethyl acetate in hexanes), pale yellow oil (˜0.12 g, not completely pure). Mixture of products was used for next step without further purification.

### Example 7. N-(trifluoroacetyl) 4-methyl-2,3,4,5-tetrahydro-1H-benzofuro[2,3-d]azepine 9 and N-(trifluoroacetyl)1-methyl-2,3,4,5-tetrahydro-1H-benzofuro[3,2-c]azepine 10

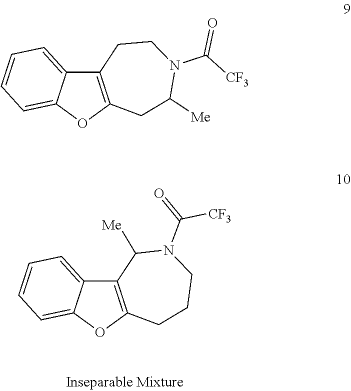 [SMILES: CC1Cc2oc3ccccc3c2CCN1C(=O)C(F)(F)F ; CC1c2c(oc3ccccc23)CCCN1C(=O)C(F)(F)F]

Compounds were synthetized according to general procedure B (0.61 mmol scale). Crude mixture of products was filtered through a plug of silica gel in dichloromethane and purified by column chromatography (10% diethyl ether in hexanes, isomers not separated). Mixture of products (˜0.14 g) was used for next step without further purification.

### Example 8. N-(trifluoroacetyl)-9-bromo-2,3,4,5-tetrahydro-1H-benzofuro[2,3-d]azepine 11 and N-(trifluoroacetyl)-9-bromo-2,3,4,5-tetrahydro-1H-benzofuro[3,2-c]azepine 12

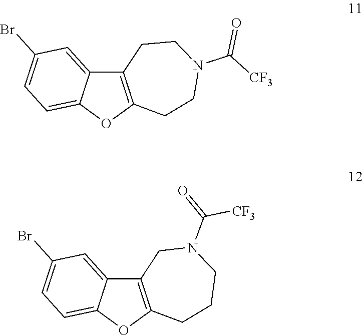 [SMILES: O=C(N1CCc2oc3ccc(Br)cc3c2CC1)C(F)(F)F ; O=C(N1CCCc2oc3ccc(Br)cc3c2C1)C(F)(F)F]

Compounds were synthesized according to general procedure B (3.6 mmol scale). Crude mixture of products was purified using column chromatography (gradient of 0 to 9% of ethyl acetate in hexanes).

Compound 11 was isolated as a tan solid (0.35 g, 27%), compound 12 as an off-white solid (0.36 g, 28%).

Compound 11

¹H NMR (400 MHz, CDCl₃) δ 7.52 (dd, J=10.7, 2.0 Hz, 1H), 7.37-7.27 (m, 1H), 7.27-7.21 (m, 1H), 3.95-3.85 (m, 4H), 3.17 (t, J=5.8 Hz, 2H), 2.95-2.86 (m, 2H).

Compound 12

¹H NMR (400 MHz, CDCl₃) δ 7.58 (dd, J=54.9, 1.9 Hz, 1H), 7.33 (m, 1H), 7.29-7.21 (m, 1H), 4.82-4.53 (m, 2H), 3.90 (q, J=5.5 Hz, 2H), 3.06 (t, J=6.8 Hz, 2H), 2.19-1.94 (m, 2H).

### Example 9. N-(trifluoroacetyl)9-hydroxy-2,3,4,5-tetrahydro-1H-benzofuro[2,3-d]azepine 13

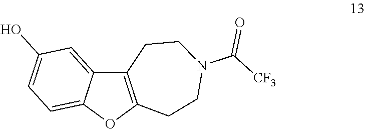 [SMILES: O=C(N1CCc2oc3ccc(O)cc3c2CC1)C(F)(F)F]

To a solution of compound 11 (150 mg, 0.41 mmol) in N,N-dimethylformamide (4.1 mL) was added bis(pinacolato)diboron (210 mg, 0.8 mmol), 1,1′-bis (diphenylphosphino) ferrocene-palladium (II) dichloride-dichloromethane complex (42 mg, 0.05 mmol) and potassium acetate (50 mg, 0.6 mmol), and the mixture was stirred at 80° C. for 13 hours. The reaction mixture was cooled to room temperature, diluted with water, and the mixture was extracted with ethyl acetate (3×) The organic layer was washed with saturated brine, dried over anhydrous sodium sulfate, filtered and concentrated. The obtained crude product was dissolved in tetrahydrofuran (4.1 mL), mixture was cooled to 0° C. (ice bath), and 1N aqueous sodium hydroxide solution (0.8 mL) and hydrogen peroxide solution (0.23 mL) were added. Reaction mixture was further stirred at room temperature for 1.5 hours, diluted with water and extracted with ethyl acetate (3×). The organic layer was washed with saturated brine, dried over anhydrous sodium sulfate, filtered and concentrated. The obtained crude product was purified using column chromatography (gradient of 16 to 33% of ethyl acetate in hexanes) to give compound 13 as a white solid (52 mg, 42%).

¹H NMR (400 MHz, CDCl₃) δ 7.21 (m, 1H), 6.80 (m, 1H), 6.75 m, 1H), 3.99-3.83 (m, 4H), 3.27-3.01 (m, 2H), 2.98-2.73 (m, 2H).

### Example 10. N-(trifluoroacetyl) 9-bromo-4-methyl-2,3,4,5-tetrahydro-1H-benzofuro[2,3-d]azepine 14 and N-(trifluoroacetyl) 9-bromo-1-methyl-2,3,4,5-tetrahydro-1H-benzofuro[3,2-c]azepine 15

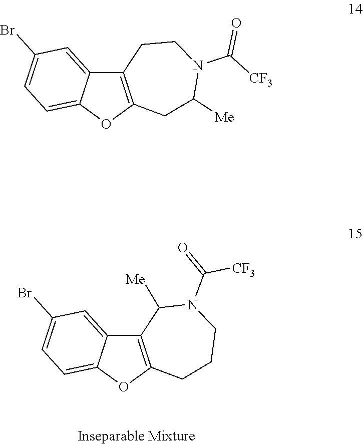 [SMILES: CC1Cc2oc3ccc(Br)cc3c2CCN1C(=O)C(F)(F)F ; CC1c2c(oc3ccc(Br)cc23)CCCN1C(=O)C(F)(F)F]

Compounds were synthesized according to general procedure B (1.4 mmol scale). Crude mixture of products was filtered through a plug of silica gel in dichloromethane and purified by column chromatography (10% diethyl ether in hexanes, isomers not separated). Mixture of products (˜0.33 g, slightly impure) was used for next step without further purification.

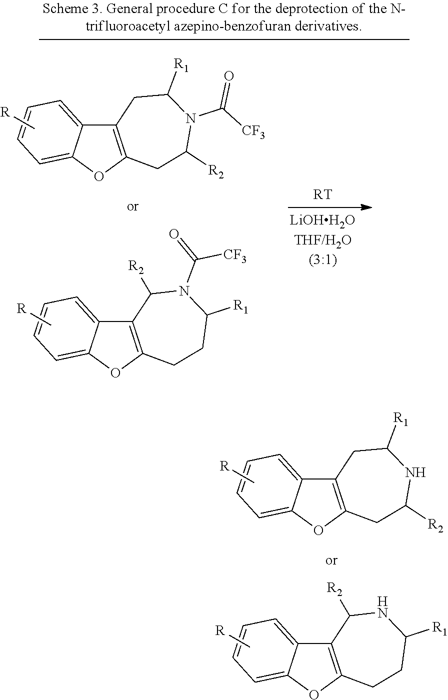

Corresponding N-(trifluoroacetyl)azepino-benzofuran derivative (1 equivalent) was dissolved in a mixture of THF:H₂O (3:1, 0.2 M) and treated with LiOH·H₂O (3 equivalents) at RT. Reaction mixture was vigorously stirred until TLC indicated full consumption of starting material (0.5 h to 21 h, as indicated for each example). Reaction mixture was diluted with brine and the mixture was extracted with DCM/iPrOH (9:1) or ethyl acetate (3×). Combined organic extracts were dried over Na₂SO₄, filtered and concentrated. Crude material was purified as specified for each example.

### Example 11. 2,3,4,5-tetrahydro-1H-benzofuro[2,3-d]azepine 16

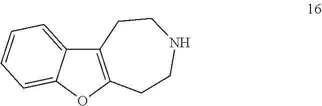 [SMILES: c1ccc2c3c(oc2c1)CCNCC3]

Reaction was performed according to general procedure C (0.22 mmol scale, 0.5 h reaction time). Crude material was purified by preparative TLC (97:3 DCM:MeOH+0.5% NH₄OH), plate was developed twice. For final purification oily compound was dissolved in diethyl ether, insoluble particles were filtered, and compound was transformed into the hydrochloride salt using 2M HCl in diethyl ether. Formed solid was sedimented by centrifugation, decanted, washed with hexanes and evaporated from MeOH.

Compound 16 HCl salt was obtained as an off-white solid (33 mg, 67%).

¹H NMR (500 MHz, CDCl₃) δ 7.44-7.33 (m, 2H), 7.25-7.16 (m, 2H), 3.17-3.07 (m, 3H), 3.07-3.00 (m, 2H), 2.82-2.75 (m, 2H), 2.09 (s, 1H). ¹³C NMR (126 MHz, CDCl₃) δ 155.4, 153.7, 130.4, 123.2, 122.2, 118.5, 115.4, 110.7, 50.4, 48.2, 32.5, 26.2. LRMS (ESI⁺) calcd. for C₁₂H₁₄NO [M+H]⁺ 188.1. found 188.1.

### Example 12. 2,3,4,5-tetrahydro-1H-benzofuro[3,2-c]azepine 17

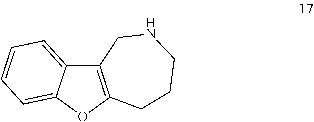 [SMILES: c1ccc2c3c(oc2c1)CCCNC3]

Reaction was performed according to general procedure C (0.2 mmol scale, 0.5 h reaction time). Crude material was purified by preparative TLC (97:3 DCM:MeOH+0.5% NH₄OH), plate was developed twice. For final purification the oily compound was dissolved in diethyl ether, insoluble particles were filtered, and compound was transformed into the hydrochloride salt using 2M HCl in diethyl ether. Formed solid was sedimented by centrifugation, decanted, washed with hexanes and evaporated from MeOH. Compound 17·HCl salt was obtained as an off-white solid (48 mg, 98%).

¹H NMR (400 MHz, CDCl₃) δ 7.42-7.32 (m, 2H), 7.24-7.12 (m, 2H), 3.97 (s, 2H), 3.21-3.13 (m, 2H), 3.05-2.97 (m, 2H), 1.93-1.83 (m, 2H), 1.67 (br, 1H). ¹³C NMR (126 MHz, CDCl₃) δ 156.1, 153.6, 129.0, 123.3, 122.3, 118.3, 113.7, 110.8, 51.3, 43.5, 28.9, 28.3. LRMS (ESI+) calcd. for C₁₂H₁₄NO [M+H]⁺ 188.1. found 188.1.

### Example 13. 4-methyl-2,3,4,5-tetrahydro-1H-benzofuro[2,3-d]azepine 18 and 1-methyl-2,3,4,5-tetrahydro-1H-benzofuro[3,2-c]azepine 19

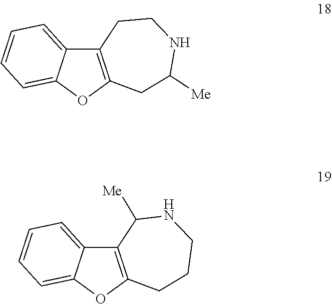 [SMILES: CC1Cc2oc3ccccc3c2CCN1 ; CC1NCCCc2oc3ccccc3c21]

Reaction was performed according to general procedure C (0.34 mmol scale, 21 h reaction time). Crude material was purified by preparative TLC (95:5 DCM:MeOH+0.5% NH₄OH). Compound 18 was isolated as a yellow oil (52 mg, 59% over two steps), compound 19 as a yellow oil (11 mg, impure <12% over two steps).

Compound 18

¹H NMR (500 MHz, CDCl₃) δ 7.43-7.38 (m, 1H), 7.38-7.34 (m, 1H), 7.23-7.16 (m, 2H), 3.46-3.37 (m, 1H), 3.07-2.95 (m, 2H), 2.95-2.88 (m, 1H), 2.88-2.78 (m, 2H), 2.76-2.68 (m, 1H), 1.86 (br, 1H), 1.27 (d, J=6.4 Hz, 3H). ¹³C NMR (126 MHz, CDCl₃) δ 154.1, 153.6, 130.4, 123.2, 122.2, 118.4, 115.4, 110.7, 53.9, 49.4, 39.4, 25.9, 23.8. LRMS (ESI+) calcd. for C₁₃H₁₆NO [M+H]⁺ 202.1. found 202.1.

Compound 19

¹H NMR (400 MHz, CDCl₃) δ 7.44-7.34 (m, 2H), 7.23-7.15 (m, 2H), 4.37 (q, J=7.0 Hz, 1H), 3.32-2.94 (m, 4H), 2.56-2.21 (m, 2H), 2.05-1.74 (m, 2H), 1.51 (d, J=6.9 Hz, 3H). LRMS (ESI+) calcd. for C₁₃H₁₆NO [M+H]⁺ 202.1. found 202.1.

### Example 14. 2-methyl-2, 3, 4, 5-tetrahydro-1H-benzofuro[2,3-d] azepine 20 and 3-methyl-2,3,4,5-tetrahydro-1H-benzofuro[3,2-c]azepine 21

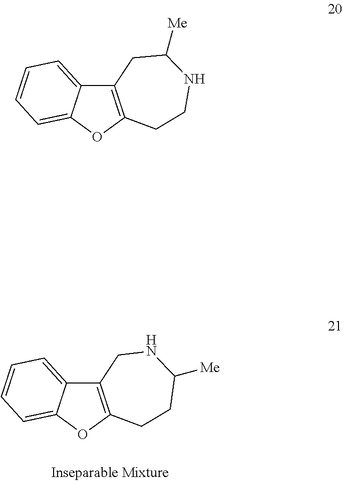 [SMILES: CC1Cc2c(oc3ccccc23)CCN1 ; CC1CCc2oc3ccccc3c2CN1]

Reaction was performed according to general procedure C (0.4 mmol scale, 2 h reaction time). Isolated crude material (59 mg) contains both isomers in ˜6:4 ratio.

¹H NMR (500 MHz, CDCl₃) δ 7.43-7.33 (m, 2H), 7.24-7.16 (m, 2H), 4.14 (d, J=15.8 Hz, 0.4H), 3.86 (dt, J=15.9, 1.4 Hz, 0.4H), 3.38-3.31 (m, 0.6H), 3.12-3.01 (m, 1.3H), 3.01-2.90 (m, 2.3H), 2.85 (dd, J=15.7, 2.4 Hz, 0.6H), 2.49 (ddd, J=15.8, 10.6, 2.2 Hz, 0.6H), 2.20 (br, 1H), 2.07-1.98 (m, 0.4H), 1.67-1.56 (m, 0.4H), 1.30 (d, J=6.4 Hz, 1.8H), 1.25 (d, J=6.6 Hz, 1.2H). LRMS (ESI+) calcd. for C₁₃H₁₆NO [M+H]⁺ 202.1. found 202.1.

### Example 15. 2,3,4,5-tetrahydro-1H-benzofuro[2,3-d]azepin-9-ol 22

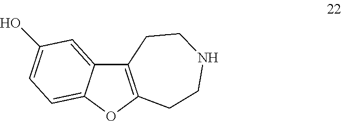 [SMILES: Oc1ccc2oc3c(c2c1)CCNCC3]

Reaction was performed according to general procedure C (0.17 mmol scale, 2 h reaction time). The crude product was purified by column chromatography (97:3 DCM:MeOH+0.5% NH₄OH) to give the free base as a waxy solid (34 mg). As final purification the compound was transformed into its hydrochloride salt. The free base was dissolved in MeOH (1.2 mL) and 2M HCl in Et₂O (0.1 mL) was added. Solution was concentrated in vacuum, the solids were washed twice with hexanes and evaporated from methanol. The hydrochloride salt of compound 22 was isolated as an off-white solid (28 mg, 74%).

¹H NMR (400 MHz, MeOD) δ 7.19 (dd, J=8.7, 0.8 Hz, 1H), 6.82 (d, J=2.5 Hz, 1H), 6.72 (m, 1H), 3.49 (q, J=5.3 Hz, 4H), 3.36-3.22 (m, 2H), 3.06-2.99 (m, 2H). ¹³C NMR (126 MHz, MeOD) δ 153.2, 152.7, 148.2, 129.6, 113.6, 112.4, 110.6, 103.0, 44.7, 25.1, 19.6. LRMS (ESI+) calcd. for C₁₂H₁₄NO₂ [M+H]⁺ 204.1. found 204.0.

### Example 16. 4-methyl-2,3,4,5-tetrahydro-1H-benzofuro[2,3-d]azepin-9-ol 23 and 9-hydroxy-1-methyl-2,3,4,5-tetrahydro-1H-benzofuro[3,2-c]azepine 24

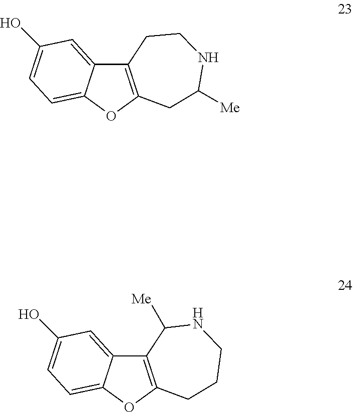 [SMILES: CC1Cc2oc3ccc(O)cc3c2CCN1 ; CC1NCCCc2oc3ccc(O)cc3c21]

To a solution of compounds 14 and 15 (188 mg, 0.5 mmol) in N,N-dimethylformamide (5.0 mL) was added bis(pinacolato)diboron (254 mg, 1.0 mmol), 1,1′-bis (diphenylphosphino) ferrocene-palladium (II) dichloride-dichloromethane complex (61 mg, 0.075 mmol) and potassium acetate (147 mg, 1.5 mmol), and the mixture was stirred at 80° C. for 16 hours. The reaction mixture was cooled to room temperature, diluted with water, and the mixture was extracted with ethyl acetate (3×). The organic layer was washed with saturated brine, dried over anhydrous sodium sulfate, filtered and concentrated. The obtained crude product was dissolved in tetrahydrofuran (5 mL), mixture was cooled to 0° C. (ice bath), and 1N aqueous sodium hydroxide solution (0.5 mL) and hydrogen peroxide solution (0.15 mL) were added. Reaction mixture was further stirred at room temperature for 2.5 hours, diluted with water and extracted with ethyl acetate (3×). The organic layer was washed with saturated brine, dried over anhydrous sodium sulfate, filtered and concentrated. The obtained crude product was filtered through a plug of silica gel in 95:5 DCM:MeOH+0.5% NH₄OH. Crude material was further dissolved in a mixture of THF:H₂O (3:1, 2.5 mL) and treated with LiOH—H₂O (63 mg, 1.5 mmol) at room temperature. Reaction mixture was vigorously stirring for 1.5 h, then sequentially diluted with equal volumes of saturated aqueous NH₄Cl and NaHCO₃ solutions. The resulting mixture was extracted with ethyl acetate, combined organic extracts were dried over Na₂SO₄, filtered and concentrated. Crude material was suspended in MeOH and cooled in ice bath, solid was collected by filtration and washed with MeOH to obtain compound 23. Methanolic fraction containing both compounds 23 and 24 was separated using preparative TLC (93:7 DCM:MeOH+0.7% NH₄OH, long development time). Compound 23 was isolated as a pale brown amorphous solid (43 mg, 26% over 4 steps) and compound 24 as a pale brown amorphous solid (13 mg, 8% over 4 steps).

Compound 23

¹H NMR (500 MHz, CD₃OD) δ 7.12 (d, J=8.7 Hz, 1H), 6.76 (d, J=2.5 Hz, 1H), 6.66 (dd, J=8.7, 2.5 Hz, 1H), 3.40-3.34 (m, 1H), 3.04-2.96 (m, 1H), 2.93 (dd, J=16.4, 2.6 Hz, 1H), 2.90-2.62 (m, 4H), 1.26 (d, J=6.3 Hz, 3H). ¹³C NMR (126 MHz, CD₃OD) δ 155.5, 154.0, 149.4, 132.2, 116.1, 112.7, 111.5, 104.2, 54.8, 49.8, 39.2, 25.6, 23.1. LRMS (ESI+) calcd. for C₁₃H₁₆NO₂ [M+H]⁺ 218.1. found 218.0.

Compound 24

¹H NMR (500 MHz, CD₃OD) δ 7.14 (d, J=8.7 Hz, 1H), 6.80 (d, J=2.4 Hz, 1H), 6.66 (dd, J=8.7, 2.5 Hz, 1H), 4.28 (q, J=7.0 Hz, 1H), 3.27-3.20 (m, 1H), 3.11-3.04 (m, 1H), 3.03-2.91 (m, 2H), 2.03-1.93 (m, 1H), 1.88-1.77 (m, 1H), 1.47 (d, J=7.0 Hz, 3H). ¹³C NMR (126 MHz, CD₃OD) δ 156.4, 154.0, 149.4, 130.6, 120.9, 112.7, 111.7, 104.4, 49.3, 44.9, 28.4, 28.3, 19.9. LRMS (ESI⁺) calcd. for C₁₃H₁₆NO₂ [M+H]⁺ 218.1. found 218.0.

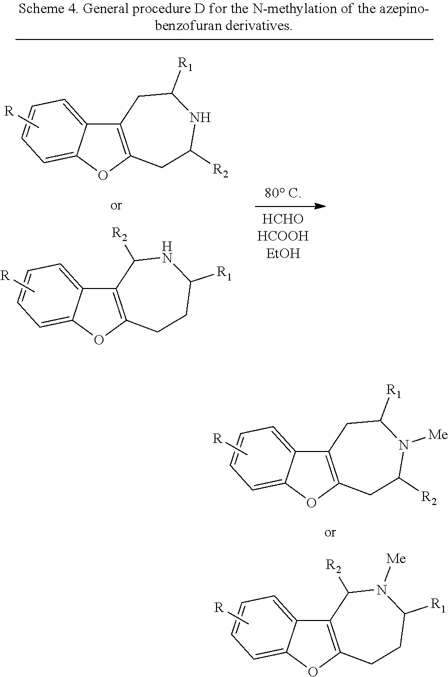

The corresponding azepino-benzofuran derivative (1 equivalent) was dissolved in EtOH (0.25 M) and treated with formaldehyde solution (aqueous 36.5%, 5 equivalents) and formic acid (10 equivalents). Reaction mixture was stirred at 80° C. for 4 hours. After cooling to RT reaction mixture was concentrated, the residue was diluted with saturated aqueous K₂CO₃ and extracted with ethyl acetate (3×). Combined organic extracts were dried over Na₂SO₄, filtered and concentrated. Crude material was purified as specified for each example.

### Example 17. 3-methyl-2,3,4,5-tetrahydro-1H-benzofuro[2,3-d]azepine 25

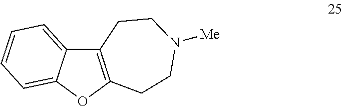 [SMILES: CN1CCc2oc3ccccc3c2CC1]

The reaction was performed according to general procedure D (0.27 mmol scale). The crude product was purified by preparative TLC (97.5:2.5 DCM:MeOH+0.25% NH₄OH, plate developed twice) to give the free base as a yellow oil (42 mg). As final purification the compound was transformed into its hydrochloride salt. The free base was dissolved in Et₂O, insoluble precipitates were filtered, and the clear solution was treated with 2M HCl in Et₂O. Formed solid was sedimented by centrifugation, decanted, washed with hexanes and evaporated from MeOH. Compound 25 was isolated as an off-white solid (45 mg, 91%).

¹H NMR (500 MHz, CDCl₃) δ 7.43-7.34 (m, 2H), 7.23-7.17 (m, 2H), 3.09-3.02 (m, 2H), 2.95-2.87 (m, 4H), 2.85-2.78 (m, 2H), 2.54 (s, 3H). ¹³C NMR (126 MHz, CDCl₃) δ 155.0, 153.7, 130.2, 123.3, 122.2, 118.4, 115.2, 110.8, 57.4, 55.2, 45.4, 28.4, 22.4. LRMS (ESI⁺) calcd. for C₁₃H₁₆NO [M+H]⁺ 202.1. found 202.1.

### Example 18. 2-methyl-2,3,4,5-tetrahydro-1H-benzofuro[3,2-c] azepine 26

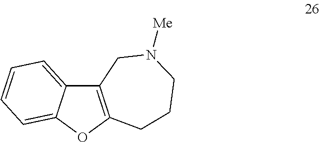 [SMILES: CN1CCCc2oc3ccccc3c2C1]

The reaction was performed according to general procedure D (0.37 mmol scale). The crude product was purified by preparative TLC (97.5:2.5 DCM:MeOH+0.25% NH₄OH, plate developed twice) to give the free base as a yellow oil (42 mg). As final purification the compound was transformed into its hydrochloride salt. The free base was dissolved in Et₂O, insoluble precipitates were filtered, and the clear solution was treated with 2M HCl in Et₂O. Formed solid was sedimented by centrifugation, decanted, washed with hexanes and evaporated from MeOH. Compound 26 was isolated as an off-white solid (74 mg, quantitative).

¹H NMR (500 MHz, CDCl₃) δ 7.40-7.33 (m, 2H), 7.22-7.16 (m, 2H), 3.75 (s, 2H), 3.01-2.91 (m, 4H), 2.51 (s, 3H), 2.00-1.92 (m, 2H).

¹³C NMR (126 MHz, CDCl₃) δ 156.1, 153.6, 129.5, 123.2, 122.3, 118.1, 113.5, 110.8, 59.7, 51.7, 45.9, 28.0, 24.8. LRMS (ESI⁺) calcd. for C₁₃H₁₆NO [M+H]⁺ 202.1. found 202.1.

### Example 19. 3,4-dimethyl-2,3,4,5-tetrahydro-1H-benzofuro[3,2-c]azepine 27

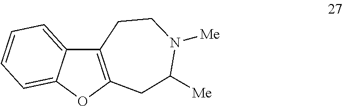 [SMILES: CC1Cc2oc3ccccc3c2CCN1C]

The reaction was performed according to general procedure D (0.37 mmol scale). The crude product was purified by preparative TLC (95:5 DCM:MeOH+0.5% NH₄OH), plate developed twice) to give the free base as a yellow oil (30 mg, 97%).

¹H NMR (500 MHz, CDCl₃) δ 7.43-7.33 (m, 2H), 7.25-7.17 (m, 2H), 3.40 (ddd, J=14.2, 5.9, 3.8 Hz, 1H), 3.37-3.29 (m, 1H), 3.16 (ddd, J=14.2, 9.5, 3.4 Hz, 1H), 3.09-2.98 (m, 2H), 2.93-2.83 (m, 1H), 2.71 (ddd, J=16.6, 6.0, 3.5 Hz, 1H), 2.50 (s, 3H), 1.26 (d, J=6.7 Hz, 3H). ¹³C NMR (126 MHz, CDCl₃) δ 153.8, 153.3, 130.2, 123.3, 122.2, 118.4, 114.8, 110.7, 56.2, 53.5, 34.2, 32.8, 19.9, 19.6. LRMS (ESI⁺) calcd. for C₁₄H₁₈NO [M+H]⁺ 216.1. found 216.0.

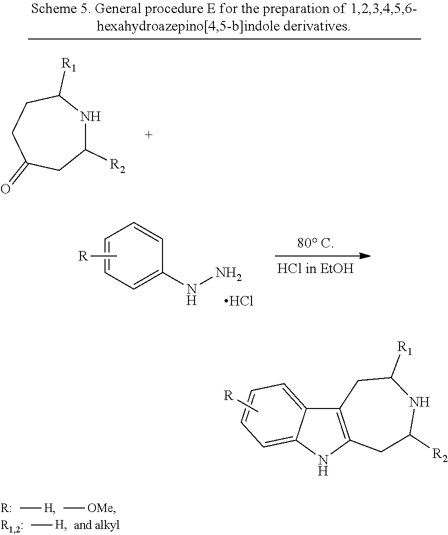

The corresponding phenyl hydrazine hydrochloride derivative (1 equivalent) and substituted azepan-4-one (1 equivalent) were dissolved in ethanol (0.1 M) and concentrated aqueous HCl (6 equivalents) was added. Dark reaction mixture was heated to 80° C. overnight, cooled to room temperature and concentrated. The oily residue was treated with 1M aqueous sodium hydroxide and mixture was extracted with dichloromethane (3×). The combined organic extracts were dried over Na₂SO₄, filtered and concentrated. Crude material was purified as specified for each example.

### Example 20. 2-methyl-1,2,3,4,5,6-hexahydroazepino[4,5-b]indole 28

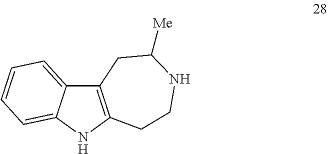 [SMILES: CC1Cc2c([nH]c3ccccc23)CCN1]

The reaction was performed according to general procedure E (0.5 mmol scale). The crude product passed through a short silica gel column (97.5:2.5 DCM:MeOH+0.25% NH₄OH to 95:5 DCM:MeOH+0.5% NH₄OH). Material was further purified by preparative TLC (95:5 DCM:MeOH+0.5% NH₄OH). As a final purification the compound was transformed into its hydrochloride salt. The free base was dissolved in methanol and the solution was acidified using aqueous HCl. Solution was concentrated in vacuum and residue was washed with hot CH₃CN to obtain compound 28 as a brown amorphous solid (58 mg, 49%).

Free Base:

¹H NMR (400 MHz, CDCl₃) δ 7.75 (s, 1H), 7.46 (dd, J=6.2, 2.5 Hz, 1H), 7.29-7.23 (m, 1H), 7.14-7.06 (m, 2H), 3.34 (ddd, J=12.8, 4.2, 3.2 Hz, 1H), 3.13-2.91 (m, 4H), 2.78 (ddd, J=15.3, 4.3, 1.8 Hz, 1H), 2.58 (ddd, J=15.3, 10.3, 1.5 Hz, 1H), 1.80 (br, 1H), 1.87-1.65 (m, 1H), 1.29 (d, J=6.4 Hz, 3H). LRMS (ESI⁺) calcd. for C₁₃H₁₇N₂ [M+H]⁺ 201.1. found 201.2.

Hydrochloride:

¹H NMR (400 MHz, MeOD) δ 7.46-7.40 (m, 1H), 7.31-7.24 (m, 1H), 7.10-6.96 (m, 2H), 3.70-3.52 (m, 2H), 3.39-3.15 (m, 4H), 3.04-2.92 (m, 1H), 1.50 (d, J=6.6 Hz, 3H).

### Example 21. 9-methoxy-2-methyl-1,2,3,4,5,6-hexahydroazepino[4,5-b]indole 29

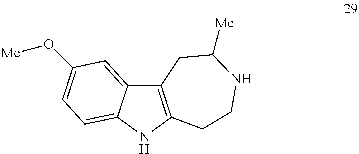 [SMILES: COc1ccc2[nH]c3c(c2c1)CC(C)NCC3]

The reaction was performed according to general procedure E (0.5 mmol scale). The crude product was purified by column chromatography (95:5 DCM:MeOH+0.5% NH₄OH to 90:10 DCM:MeOH+1% NH₄OH). Material was further purified by preparative TLC (90:10 DCM:MeOH+1% NH₄OH). For further purification the compound was transformed into its hydrochloride salt. The free base was dissolved in methanol and the solution was acidified using aqueous HCl. Solution was concentrated in vacuum to obtain compound 27 as a brown amorphous solid.

Free Base:

¹H NMR (400 MHz, CDCl₃) δ 7.67 (s, 1H), 7.14 (d, J=8.7 Hz, 1H), 6.92 (d, J=2.4 Hz, 1H), 6.76 (dd, J=8.7, 2.4 Hz, 1H), 3.86 (s, 3H), 3.38-3.25 (m, 1H), 3.11-2.90 (m, 4H), 2.74 (ddd, J=15.0, 4.3, 1.6 Hz, 1H), 2.56 (ddd, J=15.7, 10.6, 1.3 Hz, 1H), 1.90 (s, 1H), 1.29 (d, J=6.3 Hz, 3H). LRMS (ESI⁺) calcd. for C₁₄H₁₉N₂O [M+H]⁺ 231.1, found 231.1.

Hydrochloride:

¹H NMR (400 MHz, MeOD) δ 7.17 (d, J=8.8 Hz, 1H), 6.95 (d, J=2.4 Hz, 1H), 6.73 (dd, J=8.7, 2.4 Hz, 1H), 3.81 (s, 3H), 3.66-3.52 (m, 2H), 3.30-3.10 (m, 4H), 2.95 (dd, J=16.7, 9.9 Hz, 1H), 1.50 (d, J=6.5 Hz, 3H).

### Example 22. 9-methoxy-4-methyl-1,2,3,4,5,6-hexahydroazepino[4,5-b]indole 30

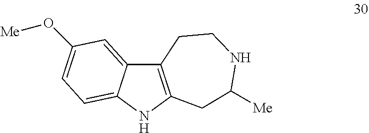 [SMILES: COc1ccc2[nH]c3c(c2c1)CCNC(C)C3]

The reaction was performed according to general procedure E (0.5 mmol scale). The crude product was purified by column chromatography (95:5 DCM:MeOH+0.5% NH₄OH to 90:10 DCM:MeOH+1% NH₄OH). Material was further purified by preparative TLC (90:10 DCM:MeOH+1% NH₄OH). For further purification the compound was transformed into its hydrochloride salt. The free base was dissolved in methanol and the solution was acidified using aqueous HCl. Solution was concentrated in vacuum to obtain compound 30 as a brown amorphous solid.

¹H NMR (400 MHz, CDCl₃) δ 7.62 (s, 1H), 7.15 (d, J=8.7 Hz, 1H), 6.91 (d, J=2.4 Hz, 1H), 6.77 (dd, J=8.7, 2.4 Hz, 1H), 3.85 (s, 3H), 3.38 (ddd, J=12.6, 4.9, 3.2 Hz, 1H), 3.09-2.99 (m, 1H), 2.98-2.80 (m, 4H), 2.80-2.71 (m, 1H), 2.19 (s, 1H), 1.25 (d, J=6.4 Hz, 3H). LRMS (ESI⁺) calcd. for C₁₄H₁₉N₂O [M+H]⁺ 231.1. found 231.1.

### Example 23. 3-(2-(3,6-dihydropyridin-1 (2H)-yl)ethyl)-5-methoxy-1H-indole 31

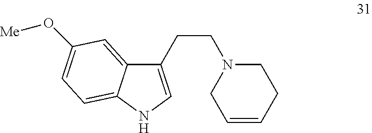 [SMILES: COc1ccc2[nH]cc(CCN3CC=CCC3)c2c1]

To a solution of 3-(2-bromoethyl)-5-methoxy-1H-indole (0.5 g, 1.97 mmol) in MeOH (anhydrous, 2.0 mL) was added 1,2,3,6-tetrahydropyridine (0.36 mL, 3.94 mmol) and the resulting mixture was stirred at 50° C. for 6 h. The reaction mixture was then concentrated in vacuum and purified by column chromatography (9:1 Ethyl acetate:MeOH+2% Et₃N) to obtain a pale yellow solid (400 mg, 79% yield).

¹H NMR (500 MHz, CDCl₃) δ 8.00 (s, 1H), 7.28-7.23 (m, 1H), 7.10 (d, J=2.5 Hz, 1H), 7.04 (d, J=2.4 Hz, 1H), 6.88 (dd, J=8.7, 2.5 Hz, 1H), 5.88-5.79 (m, 1H), 5.77-5.69 (m, 1H), 3.89 (s, 3H), 3.16 (t, J=2.8 Hz, 2H), 3.09-2.99 (m, 2H), 2.86-2.78 (m, 2H), 2.74 (t, J=5.7 Hz, 2H), 2.32-2.23 (m, 2H). LRMS (EI) calcd. for C₁₆H₂₀N₂O [M]256.2. found 256.1.

### Example 24. 11-methoxy-1,4,5,6,7,8-hexahydro-2H-3,7-methanoazonino[5,4-b]indole 32

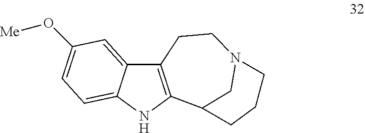 [SMILES: COc1ccc2[nH]c3c(c2c1)CCN1CCCC3C1]

A reaction flask was charged with Pd(CH₃CN)₄(BF₄)₂ (244 mg, 0.55 mmol) and CH₃CN (anhydrous, 7.0 mL) was then added to form a yellow solution. To this solution was added a solution of 3-(2-(3,6-dihydropyridin-1(2H)-yl)ethyl)-5-methoxy-1H-indole (129 mg, 0.5 mmol) in CH₃CN (anhydrous, 18.0 mL) resulting in a deep-red color. The reaction mixture was stirred for 2 h at room temperature and then warmed to 70° C. and stirred for a further 16 h. At this time, the reaction was cooled to 0° C., and MeOH (anhydrous, 4.5 mL) was added followed by NaBH₄ (61 mg, 3 equiv.), causing immediate precipitation of palladium black. The resulting black mixture was stirred for 20 min at 0° C., then diluted with Et₂O (50 mL), and filtered through Celite, and the filter cake was washed with additional Et₂O (4×10 mL). The combined filtrate and washings were concentrated to afford the crude product. The product was purified by column chromatography (98:2 DCM:MeOH+2% Et₃N).

¹H NMR (500 MHz, CDCl₃) δ 7.19 (d, J=8.7 Hz, 1H), 6.93 (d, J=2.4 Hz, 1H), 6.80 (d, J=6.3 Hz, 1H), 3.86 (s, 3H), 3.43 (dt, J=13.6, 4.1 Hz, 1H), 3.36-3.27 (m, 4H), 3.20-3.14 (m, 2H), 2.97-2.93 (m, 2H), 2.02-1.96 (m, 1H), 1.90 (tt, J=13.0, 4.1 Hz, 1H), 1.84-1.70 (m, 1H), 1.39-1.32 (m, 1H). LRMS (EI) calcd. for C₁₆H₂₀N₂O [M]⁺ 256.2. found 256.1.

### Example 25. 9-methoxy-4-propyl-1,2,3,4,5,6-hexahydroazepino[4,5-b]indol-5-yl acetate 33

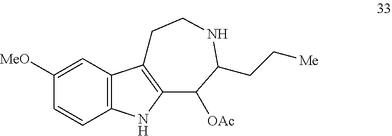 [SMILES: CCCC1NCCc2c([nH]c3ccc(OC)cc23)C1OC(C)=O]

In a screw top vial 5-methoxytryptamine hydrochloride (100 mg, 0.55 mmol) was dissolved in glacial acetic acid (3 mL) and 2-bromovaleraldehyde (101 mg, 0.63 mmol) was added. Reaction mixture was stirred for 90 minutes at 80° C., afterwards it was cooled to RT and quenched by addition of saturated sodium bicarbonate (until pH of ˜8 was reached). Mixture was extracted twice with DCM, combined organic extracts were dried over Na₂SO₄, filtered and concentrated. The crude product was purified by preparative TLC (50:50 hexane:ethyl acetate+2% triethylamine, Rf=0.24). Product was isolated as a brown solid (12 mg, 6.9% yield).

¹H NMR (500 MHz, CDCl₃) δ 8.37 (s, 1H), 7.22 (d, J=8.8 Hz, 1H), 6.96 (d, J=2.4 Hz, 1H), 6.88 (dd, J=8.8, 2.4 Hz, 1H), 5.81 (d, J=1.5 Hz, 1H), 3.87 (s, 4H), 3.53 (m, 1H), 3.18 (m, 1H), 3.05 (m, 1H), 3.01-2.93 (m, 2H), 2.87 (q, J=7.2 Hz, 1H), 2.10 (d, J=0.8 Hz, 3H), 1.61-1.51 (m, 2H), 1.51-1.35 (m, 2H), 0.98 (t, J=7.2 Hz, 3H).

LRMS (ESI⁺) calcd. for C₁₆H₂₁N₂O [M-OAc]⁺257.2. found 257.2.

### Example 26. 3,4-dimethyl-2,3,4,5-tetrahydro-1H-benzofuro[2,3-d]azepin-9-ol 34

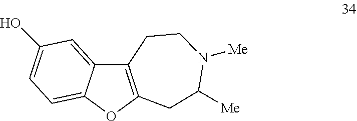 [SMILES: CC1Cc2oc3ccc(O)cc3c2CCN1C]

The reaction was performed according to general procedure D (0.217 mmol scale). The crude product was purified by preparative TLC (95:5 DCM:MeOH+0.5% NH₄OH). The slightly impure free base was dissolved in MeOH, acidified with aq. HCl (12.1 M), and repeatedly concentrated from MeOH. The hydrochloride salt was further purified by preparative TLC (90:10 DCM:MeOH+0.1% aq. 12.1 M HCl) to give the product 34 as a beige amorphous solid (30 mg, 52%).

¹H NMR (500 MHz, MeOD) δ 7.19 (d, J=8.8 Hz, 1H), 6.83 (d, J=2.5 Hz, 1H), 6.73 (dd, J=8.8, 2.5 Hz, 1H), 3.89-3.80 (m, 1H), 3.63 (dt, J=13.8, 5.5 Hz, 1H), 3.50 (dt, J=13.7, 5.9 Hz, 1H), 3.33-3.27 (m, 1H), 3.22-3.14 (m, 1H), 3.01 (t, J=5.8 Hz, 2H), 2.91 (s, 3H), 1.43 (d, J=6.8 Hz, 3H). ¹³C NMR (126 MHz, MeOD) δ 154.5, 152.5, 149.7, 130.9, 114.5, 113.7, 112.0, 104.4, 60.5, 54.3, 36.7, 31.2, 18.8, 17.1.δ 153.8, 153.3, 130.2, 123.3, 122.2, 118.4, 114.8, 110.7, 56.2, 53.5, 34.2, 32.8, 19.9, 19.6. LRMS (ESI⁺) calcd. for C₁₄H₁₈NO₂ [M+H]⁺ 232.1. found 232.0.

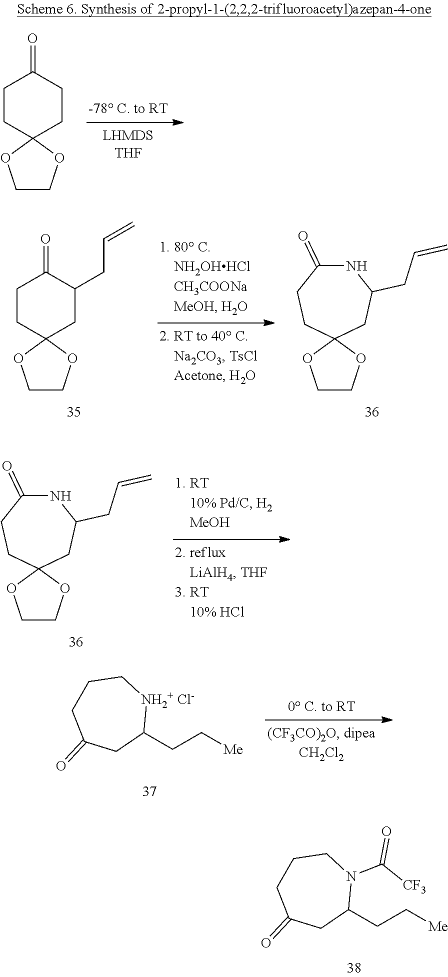

### Example 27. 7-allyl-1,4-dioxaspiro[4.5]decan-8-one 35

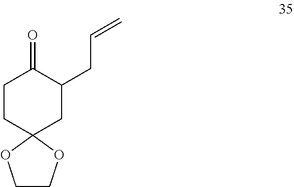

Solution of 1,4-Cyclohexanedione monoethylene acetal (7.81 g, 50 mmol) in THF (0.5 M, 100 mL) was cooled to −78° C. and LHMDS (1M in THF, 50 mL) was added in two portions via cannula. The reaction mixture was further stirred for 20 min, then allyl bromide was added dropwise over 5 min and reaction was allowed to slowly warm to room temperature. After 15.5 h reaction was quenched with sat. NH₄Cl solution (50 mL), phases were separated, and aq. Phase further extracted with Et₂O (3×20 mL), combined extracts were washed with brine (50 mL), dried over Na₂SO₄, filtered and concentrated onto celite. Crude material was purified by column chromatography using gradient of AcOEt in hexanes (2 to 10%). Compound was isolated as a pale-yellow oil (4.85 g, 49%) with spectral characterization identical to the literature values (Ibrahem, I. & Córdova, A. 2006).

¹H NMR (500 MHz, CDCl₃) δ 5.82-5.69 (m, 1H), 5.05-4.96 (m, 2H), 4.07-3.97 (m, 4H), 2.77-2.58 (m, 2H), 2.58-2.49 (m, 1H), 2.40-2.34 (m, 1H), 2.14-1.90 (m, 4H), 1.73-1.65 (m, 1H).

### Example 28. 7-allyl-1,4-dioxa-8-azaspiro[4.6]undecan-9-one 36

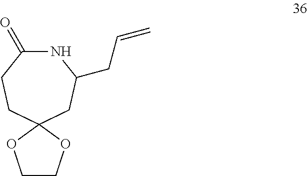

Solid reagents were added to the solution of compound 35 (6.26 g, 31.9 mmol) in MeOH (59.3 mL and H₂O (14.7 mL) and the reaction mixture was heated to 80° C. After 3 h MeOH was evaporated under reduced pressure, mixture was diluted with brine (20 mL) and extracted with DCM (3×50 mL). Combined extracts were dried over Na₂SO₄, filtered and concentrated to a yellow oil that slowly crystallized. Crude oxime was dissolved in a mixture of acetone (128 mL) and H₂O (192 mL), Na₂CO₃ and TsCl were added, and the reaction mixture was stirred 2 h at RT and 20 h at 50° C. Oxime was not fully consumed, additional TsCl (3.04 g, 15.95 mmol) and Na₂CO₃ were added (2.54 g, 23.92 mmol) and the reaction was further stirred at 40° C. After 47 h acetone was evaporated under reduce pressure and the aqueous mixture was extracted with DCM:iPrOH 9:1 (200 mL, 3×100 mL, after 2nd extraction the aqueous phase was saturated with NaCl). Combined extracts were dried over Na₂SO₄, filtered and concentrated. The crude material (beige solid, 6.72 g) was purified by short column chromatography using 50% AcOEt in hexanes (1:1) to 50% acetone in hexanes (1:1) to acetone. Initial mixed fractions (2.08 g) contained product and oxime intermediate (˜1:1), later fractions only pure product (4.41 g) Mixture of starting oxime and product was further dissolved in acetone (39.5 mL) and H₂O (59.3 mL). Na₂CO₃ (3.13 g) and TsCl (2.81 g) were added, and the reaction was stirred 2 h at RT and overnight at 40° C. Acetone was evaporated and aqueous residue extracted with DCM:iPrOH 9:1 (3×30 mL), combined extracts were dried over Na₂SO₄, filtered and concentrated. Material was purified by a short column chromatography in 50% AcOEt in hexanes (1:1) to 50% acetone in hexanes (1:1) to acetone. Compound 36 was obtained as an off-white amorphous solid (5.61 g, 83% over two steps).

¹H NMR (500 MHz, CDCl₃) δ 5.87-5.66 (m, 2H), 5.21-5.12 (m, 2H), 4.01-3.90 (m, 4H), 3.68-3.60 (m, 1H), 2.66 (ddd, J=14.8, 13.1, 2.2 Hz, 1H), 2.39-2.19 (m, 3H), 1.92-1.79 (m, 3H), 1.71-1.64 (m, 1H). ¹³C NMR (126 MHz, CDCl3) δ 176.8, 133.3, 119.6, 109.0, 64.9, 64.6, 47.8, 45.2, 40.3, 32.9, 31.2.

### Example 29. 2-propylazepan-4-one hydrochloride 37

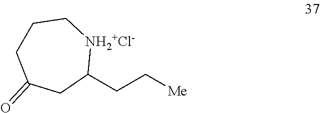

Amide 36 (2.11 g, 10.0 mmol) was dissolved in MeOH (40 mL, 0.25 M, not dry) and moist 10% Pd on C (212 mg) was added. Reaction mixture was stirred under hydrogen atmosphere (50 PSI), after 5.5 h suspension was filtered through a paper filter, filter was rinsed with MeOH, and solution was concentrated to obtain a white solid. Dried crude product (2.09 g) was dissolved in THF (39.2 mL, 0.25M), cooled in ice bath (0° C.) and LiAlH₄ (1.86 g, 49.0 mmol) was carefully added in small portions at first, after exothermic reaction subsided, the entire remaining portion was added to the suspension (starting material precipitates from cold solution). Reaction mixture was allowed to warm to room temperature and after 1 h heated to reflux for 4 h. Reaction mixture was further cooled in ice bath and quenched slowly by addition of H₂O (1.9 mL), 15% aq. NaOH (1.9 mL) and H₂O (5.6 mL), thick suspension was diluted with THF (20 mL), dried over Na₂SO₄ and stirred until all salts were white and loose. Solid was filtered off and rinsed with THF 4×(30 mL). Combined THF washings were acidified with aq. HCl (12.1M, 1 mL) and org. volatiles were evaporated under reduced pressure. To the residue was added 10% HCl (42 mL) and the mixture was further stirred at room temperature for 2.5 days. Solution was concentrated under reduced pressure and the resulting oil was washed with Et₂O (3×40 mL) and evaporated from MeOH. Compound was obtained as a pale brown oil (2.26 g) and used without further purification.

### Example 30. 2-propylazepan-4-one hydrochloride 38

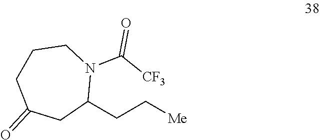 [SMILES: CCCC1CC(=O)CCCN1C(=O)C(F)(F)F]

Crude 2-propylazepan-4-one hydrochloride 37 (˜1.44 g, <7.5 mmol) was dissolved in CH₂Cl₂ (25 mL, 0.3M) after addition of dipea (3.92 mL, 22.5 mmol). Solution was cooled in ice bath and (CF₃CO)₂O (2.09 mL, 15 mmol) was added dropwise. Reaction was warmed to room temperature, stirred for 24 h and quenched by pouring into sat. NaHCO₃ solution (50 mL). Phases were separated and aq. phase further extracted with DCM (2×20 mL), combined extracts were dried over Na₂SO₄, filtered and concentrated. Crude material was purified by column chromatography using a gradient of AcOEt in hexanes 1:4, 1:3 to 1:2. Compound was isolated as a yellow oil (1.27 g). NMR spectra are complicated due to the presence of rotamers.

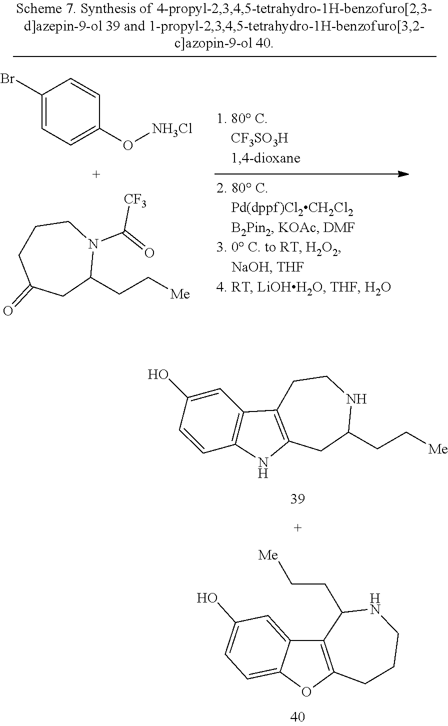

### Example 31. 4-propyl-2,3,4,5-tetrahydro-1H-benzofuro[2,3-d]azepin-9-ol 39 and 1-propyl-2,3,4,5-tetrahydro-1H-benzofuro[3,2-c]azepin-9-ol

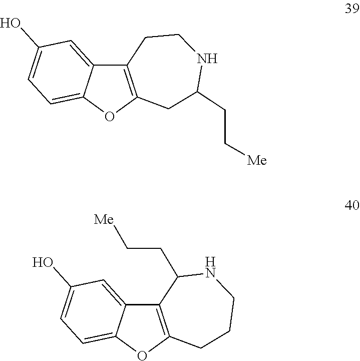 [SMILES: CCCC1Cc2oc3ccc(O)cc3c2CCN1 ; CCCC1NCCCc2oc3ccc(O)cc3c21]

O-(4-bromophenyl) hydroxylamine hydrochloride (1.12 g, 5.0 mmol) and 2-propyl-1-(2,2,2-trifluoroacetyl)azepan-4-one (1.26 g, 5.0 mmol) were combined in 1,4-dioxane (10 μmL) and warmed to 80° C. methanesulfonic acid was added after 10 min to the hot mixture and reaction continued 6 h. After cooling to room temperature, the dark mixture was added into sat. solution of NaHCO₃ (40 mL) and extracted with Et₂O (3×15 mL), combined extracts were dried over Na₂SO₄, filtered and concentrated on celite. Material was purified by column chromatography using a gradient of AcOEt in hexanes 5%, 10% to 20%. Inseparable mixture of bromide intermediates was obtained as an orange oil (0.67 g, 33%). NMR spectra are complicated (mixture of isomers, presence of rotamers).

Mixture of bromide intermediates was combined with solid reagents in DMF (16.6 mL, 0.1M) and the reaction mixture was heated to 80° C. After 15 h reaction was cooled to RT, diluted with Et₂O (50 mL), washed with H₂O (3×50 mL) and brine (poor separation of phases, filtered through sand and cotton, rinsed with AcOEt). After filtration phases were separated and combined org. extracts were dried over Na₂SO₄, filtered and concentrated.

Crude dark brown oil was diluted with THF (not anhydrous, 16.6 mL, 0.1 M), cooled in ice bath to 0° C. NaOH solution (1 M, 1.66 mL) and 30% H₂O₂ (509 uL, 4.98 mmol) were added and the reaction was further stirred at room temperature for 1.5 h. Reaction was diluted with H₂O (4.2 mL) and LiOH·H₂O (209 mg, 9.96 mmol) was added, and the mixture was stirred at RT 16 h (only partial reaction). Additional H₂O (4.2 mL) and LiOH·H₂O (209 mg, 9.96 mmol) were added, and the mixture was stirred vigorously 4 h. Reaction was diluted with sat. NH₄Cl solution (10 mL) and sat. NaHCO₃ solution (20 mL). Mixture was then diluted with AcOEt (30 mL), phases were separated and aq. phase was further extracted with DCM:iPrOH 9:1 (6×30 mL). Combined extracts were dried over Na₂SO₄, filtered and concentrated on celite. Crude product was purified two times by column chromatography using DCM:MeOH:NH4OH 95:5:0.5. Brown solid (310 mg) containing both isomers was further purified using preparative TLC (DCM:MeOH:NH4OH 95:5:0.5, plate developed twice). Compounds 39 and 40 were obtained as brown amorphous solids. The slightly impure free bases were dissolved in MeOH, acidified with aq. HCl (12.1 M) and repeatedly concentrated from EtOH. The hydrochloride salt of compound 39 was suspended in MeOH, chilled in freezer (−15° C.), solid were sedimented by centrifugation and solvent siphoned off. Washing was repeated with MeOH and Et₂O to obtain a pure fraction of the product 39 as a beige amorphous solid (144 mg). Impure fractions of compound 39 (142 mg) and compound 40 (45 mg) were also obtained.

Compound 39

¹H NMR (500 MHz, CD₃OD) δ 7.21 (d, J=8.7 Hz, 1H), 6.83 (d, J=2.4 Hz, 1H), 6.74 (dd, J=8.8, 2.5 Hz, 1H), 3.71-3.64 (m, 1H), 3.61-3.53 (m, 1H), 3.41-3.35 (m, 1H), 3.33-3.27 (m, 1H), 3.18-3.11 (m, 1H), 3.11-2.95 (m, 2H), 1.85-1.65 (m, 2H), 1.62-1.44 (m, 2H), 1.03 (t, J=7.3 Hz, 3H). ¹³C NMR (126 MHz, CD₃OD) δ 153.1, 151.6, 148.2, 129.5, 113.6, 112.4, 110.6, 103.0, 57.1, 45.9, 35.1, 29.8, 19.5, 18.3, 12.7. LRMS (ESI⁺) calcd. for C₁₅H₂₀NO₂ [M+H]⁺ 246.2. found 246.2.

### Example 32. 2,2,2-trifluoro-1-(9-fluoro-1,2,4,5-tetrahydro-3H-benzofuro[2,3-d]azepin-3-yl)ethan-1-one 41 and 2,2,2-trifluoro-1-(9-fluoro-1,3,4,5-tetrahydro-2H-benzofuro[3,2-c]azepin-2-yl)ethan-1-one 42

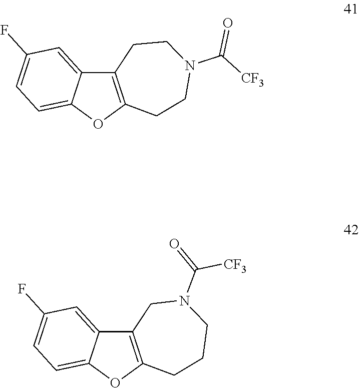 [SMILES: O=C(N1CCc2oc3ccc(F)cc3c2CC1)C(F)(F)F ; O=C(N1CCCc2oc3ccc(F)cc3c2C1)C(F)(F)F]

Compounds were synthesized according to general procedure B (1.3 mmol scale). Crude mixture of products was filtered through a plug of silica in CH₂Cl₂ and separated using a preparative TLC (10% ethyl acetate in hexanes). Compound 41 was isolated as a yellow oil (110 mg, 28%), and compound 42 as a pale-yellow solid (136 mg, 34%).

Compound 41

¹H NMR (400 MHz, CDCl₃) δ 7.35-7.28 (m, 1H), 7.10-7.01 (m, 1H), 7.01-6.93 (m, 1H), 3.98-3.86 (m, 4H), 3.23-3.14 (m, 2H), 2.98-2.85 (m, 2H).

Compound 42

¹H NMR (400 MHz, CDCl3) δ 7.35-7.28 (m, 1H), 7.21-7.03 (m, 1H), 7.00-6.91 (m, 1H), 4.78-4.61 (m, 2H), 3.96-3.86 (m, 2H), 3.13-3.01 (m, 2H), 2.19-2.07 (m, 2H).

### Example 33. 2,2,2-trifluoro-1-(9-fluoro-1,2,4,5-tetrahydro-3H-benzofuro[2,3-d]azepin-3-yl)ethan-1-one 43

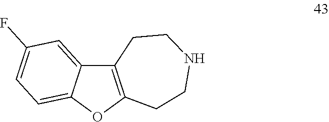 [SMILES: Fc1ccc2oc3c(c2c1)CCNCC3]

Reaction was performed according to general procedure C (0.37 mmol scale, 0.5 h reaction time). Crude material was purified by preparative TLC (95:5:0.5 DCM:MeOH:NH₄OH). For final purification oily compound was dissolved in methanol and transformed into the hydrochloride salt using aq. HCl (12.1M) and repeatedly concentrated from methanol under reduced pressure. Solid material was further washed with Et₂O, sedimented by centrifugation and solvent decanted. Compound 43·HCl salt was obtained as an off-white solid (80 mg, 91%).

¹H NMR (500 MHz, CD₃OD) δ 7.40 (dd, J=8.9, 4.0 Hz, 1H), 7.24 (dd, J=8.7, 2.6 Hz, 1H), 7.02 (td, J=9.1, 2.7 Hz, 1H), 3.57-3.49 (m, 4H), 3.36-3.29 (m, 2H), 3.12-3.07 (m, 2H). 19F NMR (376 MHz, CD₃OD) δ −121.80. ¹³C {¹H, ¹⁹F} NMR (101 MHz, CD₃OD) δ 160.8, 155.8, 151.4, 131.3, 115.7, 112.7, 112.6, 105.4, 48.4, 46.0, 26.5, 20.9. LRMS (ESI⁺) calcd. for C₁₂H₁₃FNO [M+H]⁺ 206.1, 206.3.

### Example 34. 2,2,2-trifluoro-1-(9-fluoro-1,2,4,5-tetrahydro-3H-benzofuro[2,3-d]azepin-3-yl)ethan-1-one 44

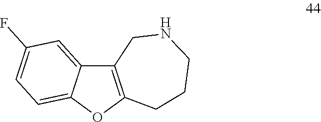 [SMILES: Fc1ccc2oc3c(c2c1)CNCCC3]

Reaction was performed according to general procedure C (0.45 mmol scale, 0.5 h reaction time). Crude material was purified by preparative TLC (95:5:0.5 DCM:MeOH:NH₄OH). For final purification oily compound was dissolved in methanol and transformed into the hydrochloride salt using aq. HCl (12.1M) and repeatedly concentrated from methanol under reduced pressure. Solid material was further washed with Et₂O, sedimented by centrifugation and solvent decanted. Compound 44·HCl salt was obtained as an off-white solid (97 mg, 89%).

1H NMR (500 MHz, CD₃OD) δ 7.44 (dd, J=8.9, 4.0 Hz, 1H), 7.31 (dd, J=8.7, 2.6 Hz, 1H), 7.04 (td, J=9.1, 2.6 Hz, 1H), 4.43 (s, 2H), 3.65-3.53 (m, 2H), 3.20-3.12 (m, 2H), 2.23-2.13 (m, 2H). ¹⁹F NMR (376 MHz, CD₃OD) 5-121.17. ¹³C NMR (101 MHz, CD₃OD) δ 160.9, 160.8, 151.3, 130.2, 113.0, 112.8, 110.2, 105.2, 50.5, 41.6, 28.0, 23.8. LRMS (ESI⁺) calcd. for C₁₂H₁₃FNO [M+H]⁺ 206.1, 206.3.

### Example 35. 4-propyl-1,2,3,4,5,6-hexahydroazepino[4,5-b]indole 45

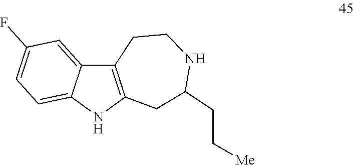 [SMILES: CCCC1Cc2[nH]c3ccc(F)cc3c2CCN1]

The reaction was performed according to general procedure E (0.5 mmol scale). The crude product was purified using a silica gel column chromatography (95:5:0.5 DCM:MeOH:NH₄OH). Material was further purified by preparative TLC (95:5:0.5 DCM:MeOH:NH₄OH). For final purification the compound was transformed into its hydrochloride salt in methanol using aqueous HCl (12.1 M). The solution was concentrated in vacuum and residue was suspended in CH₃CN, solid collected by filtration and washed with CH₃CN to obtain compound 45 as a orange amorphous solid (47 mg, 36%).

¹H NMR (500 MHz, CD₃OD) δ 7.42 (dt, J=7.8, 1.1 Hz, 1H), 7.28 (dt, J=8.1, 0.9 Hz, 1H), 7.10-7.03 (m, 1H), 7.04-6.98 (m, 1H), 3.62-3.55 (m, 1H), 3.55-3.48 (m, 1H), 3.34-3.25 (m, 2H), 3.23-3.09 (m, 3H), 1.79-1.67 (m, 2H), 1.63-1.46 (m, 2H), 1.01 (t, J=7.3 Hz, 3H). ¹³C NMR (126 MHz, CD₃OD) δ 136.5, 133.4, 129.3, 122.3, 120.1, 118.2, 111.8, 110.6, 58.9, 47.4, 36.0, 30.5, 22.3, 19.9, 14.1. LRMS (ESI⁺) calcd. for C₁₅H₂₁N₂[M+H]⁺ 229.2. found 229.3.

### Example 36. 9-methoxy-4-propyl-1,2,3,4,5,6-hexahydroazepino[4,5-b]indole 46

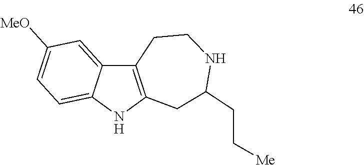 [SMILES: CCCC1Cc2[nH]c3ccc(OC)cc3c2CCN1]

The reaction was performed according to general procedure E (0.5 mmol scale). The crude product was purified using a silica gel column chromatography (95:5:0.5 DCM:MeOH:NH₄OH). Material was further purified by preparative TLC (95:5:0.5 DCM:MeOH:NH₄OH). As a final purification the compound was transformed into its hydrochloride salt in methanol using aqueous HCl (12.1 M). Solution was concentrated in vacuum and residue was suspended in CH₃CN, solid collected by filtration and washed with CH₃CN to obtain compound 46 as a brown amorphous solid (59 mg, 40%).

¹H NMR (400 MHz, CD₃OD) δ 7.17 (d, J=8.7 Hz, 1H), 6.94 (d, J=2.4 Hz, 1H), 6.74 (dd, J=8.7, 2.4 Hz, 1H), 3.81 (s, 3H), 3.64-3.47 (m, 2H), 3.35 (d, J=2.9 Hz, 1H), 3.25 (dd, J=16.7, 2.6 Hz, 1H), 3.22-3.05 (m, 3H), 1.76-1.66 (m, 2H), 1.64-1.44 (m, 2H), 1.02 (t, J=7.3 Hz, 3H). ¹³C NMR (126 MHz, CD₃OD) δ 155.4, 134.1, 131.7, 129.6, 112.5, 112.3, 110.5, 100.5, 58.9, 56.3, 47.4, 36.0, 30.6, 22.4, 19.9, 14.1. LRMS (ESI⁺) calcd. for C₁₆H₂₃N₂O [M+H]⁺ 259.2. found 259.4.

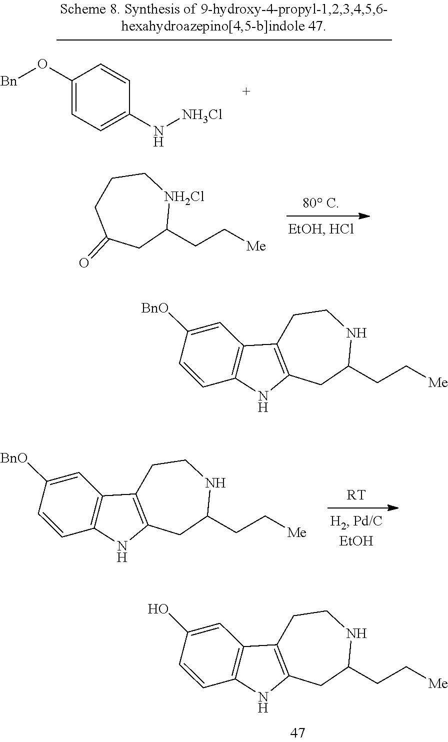

### Example 37. 9-hydroxy-4-propyl-1,2,3,4,5,6-hexahydroazepino[4,5-b]indole 47

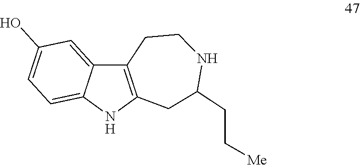 [SMILES: CCCC1Cc2[nH]c3ccc(O)cc3c2CCN1]

Benzyloxy intermediate was prepare according to general procedure E (1.0 mmol scale). The crude product was purified using a silica gel column chromatography (95:5:0.5 DCM:MeOH:NH₄OH). The obtained red amorphous solid (180 mg) was dissolved in EtOH (2.7 mL), 10% Pd on carbon (50 mg) was added and the suspension was stirred under hydrogen atmosphere (50 psi). After 4 h, due to the low conversion, additional EtOH (2.7 mL), 10% Pd/C (50 mg) and aq. HCl (12.1M, 67 μL, 1.5 eq.) were added and stirring under hydrogen atmosphere continued. Benzyloxy intermediate was still present 15 h after the start of the reaction, and additional 10% Pd/C (50 mg) was added, and reaction continued. After 23 h suspension was filtered through filter paper (filter washed with EtOH), EtOH solution was basified with aq. NH₃ solution and concentrated under reduced pressure onto celite. Crude material was purified by column chromatography using DCM:MeOH:NH₄OH 90:10:1. As final purification step the compound was transformed into its hydrochloride salt in methanol using aqueous HCl (12.1 M). Solution was concentrated in vacuum and residue was suspended in CH₃CN, solid collected by filtration and washed with CH₃CN to obtain compound 47 as a dark brown amorphous solid 108 mg, 39% over two steps).

¹H NMR (500 MHz, CD₃OD) δ 7.11 (d, J=8.6 Hz, 1H), 6.80 (d, J=2.3 Hz, 1H), 6.66-6.62 (m, 1H), 3.60-3.53 (m, 1H), 3.53-3.47 (m, 1H), 3.31-3.26 (m, 1H), 3.23 (dd, J=16.7, 2.5 Hz, 1H), 3.14-3.01 (m, 3H), 1.75-1.68 (m, 2H), 1.60-1.45 (m, 2H), 1.01 (t, J=7.3 Hz, 3H). ¹³C NMR (126 MHz, CD₃OD) δ 151.6, 134.1, 131.4, 130.0, 112.3, 112.2, 109.9, 102.7, 58.9, 47.3, 36.0, 30.6, 22.4, 19.9, 14.1. LRMS (ESI⁺) calcd. for C₁₅H₂₁N₂O [M+H]⁺ 245.2. found 245.4.

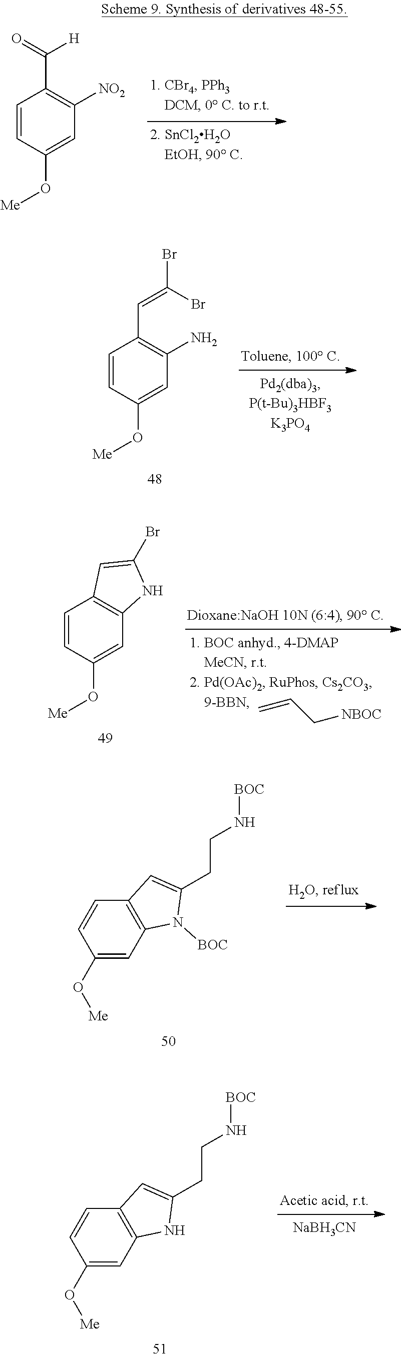

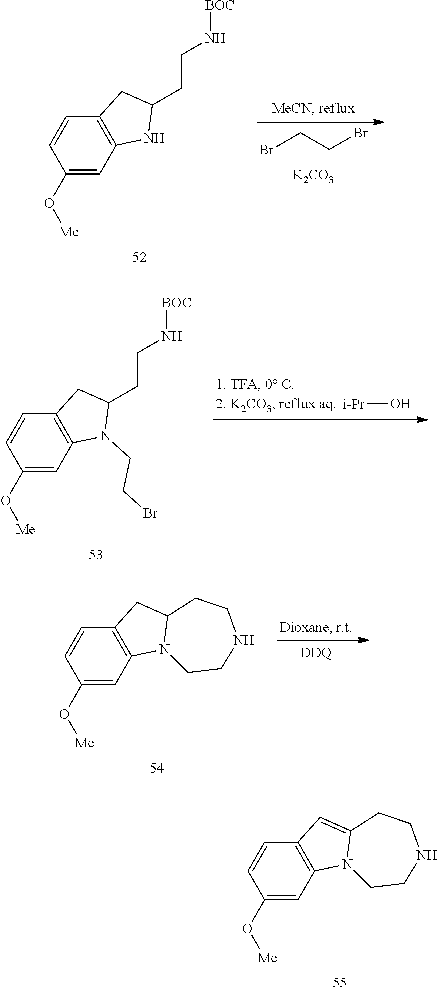

### Example 38. 2-(2,2-dibromovinyl)-5-methoxyaniline 48

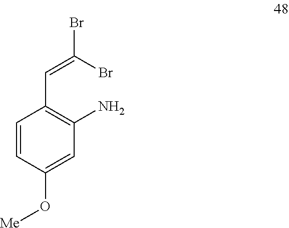 [SMILES: COc1ccc(C=C(Br)Br)c(N)c1]

To a solution of 4-methoxy-2-nitrobenzaldehyde (5.0 g, 27.6 mmol, 1 equiv) and CBr₄ (13.73 g, 41.14 mmol, 1.5 equiv) in DCM (140 ml, 0.2 M with respect to the aldehyde) at 0° C. was added a solution of PPh₃ (21.72 g, 262.29 mmol, 3 equiv) in DCM (90 ml, 0.9 M) dropwise over a 30-minute period, maintaining the temperature under 5° C. The reaction was stirred for 30 min before warming to room temperature, and stirred for an additional 30 min. Consumption of the starting material was monitored by TLC. The reaction was filtered through a short pad of silica gel, eluting with 10% EtOAc in hexanes until all product was collected as monitored by TLC to reduce the amount of triphenylphosphine oxide present. The crude was concentrated to an oil, taken up into EtOH (90 ml, 0.3 M) and SnCl₂·H₂O (26.96 g, 129.83 mmol, 5 equiv) was added. The reaction was refluxed at 90° C. for 45 min, cooled, and was basified using K₂CO₃ to pH 10. The aqueous layer was extracted 5 times with EtOAc, and the combined organic extracts were washed with H₂O, brine and dried over Na₂SO₄. The product was purified by silica gel flash chromatography (silica gel, 20% ethyl acetate in hexanes). Product was obtained as a yellow solid (7.1 g, 83% for 2 steps), spectral data match the literature report (Zeidan, N. et al. 2017).

### Example 39. 2-bromo-6-methoxy-1H-indole 49

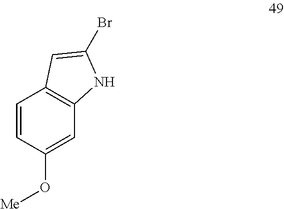 [SMILES: COc1ccc2cc(Br)[nH]c2c1]

To a round bottle flask was added compound 48 (4.8 g, 15.64 mmol), Pd₂ (dba)₃ (429 mg, 0.469 mmol, 3 mol %), PtBu₃HBF₄ (670 mg, 671 mmol. 30 mol), and K₃PO₄ (14.73 g, 69.38 mmol, 3 equiv). The flask was purged with argon, followed by the addition of toluene (78 mL, 0.2 M). The flask was sealed and heated to 100° C. and stirred vigorously for 16 hours. The vessel was cooled to room temperature and the contents added directly on to a flash column, (silica gel, 10% ethyl acetate in hexanes). Product 49 was obtained as a light red oil (2.55 g, 72%) (Newman, S. G. et al. 2009).

¹H NMR (500 MHz, CDCl₃) δ 7.93 (s, 1H), 7.39 (d, J=8.4 Hz, 1H), 6.85-6.69 (m, 2H), 6.45 (s, 1H), 3.85 (s, 3H). ¹³C NMR (500 MHz, CDCl₃) δ 120.28, 110.26, 104.69, 94.22, 55.68. LRMS (EI) calcd. For C₉H₈BrNO 225.0 [M]⁺. found: 225.0.

### Example 40. tert-butyl 2-(2-((tert-butoxycarbonyl)amino)ethyl)-6-methoxy-1H-indole-1-carboxylate 50

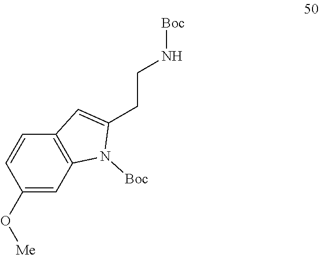 [SMILES: COc1ccc2cc(CCNC(=O)OC(C)(C)C)n(C(=O)OC(C)(C)C)c2c1]

To a solution of 49 (2.48 g, 10.87 mmol, 1 equiv) in acetonitrile (22 mL, 0.5 M) was successively added DMAP (265 mg, 2.17 mmol. 0.2 equiv) and Boc₂O (2.61 g, 11.96 mmol, 1.1 equiv) at room temperature. The mixture was stirred for 12 h, diluted with DCM (50 mL), and hydrolyzed with water (25 mL). The aqueous layer was extracted with DCM (×3), and the combined organic layers were dried with Na₂SO₄, filtered and concentrated. The residue was then filtered over silica gel and continued directly to the next step.

A solution of tert-butyl allylcarbamate (2.67 g, 16.37 mmol, 2 eq.) in 1,4 dioxane (10 mL) was cooled in an ice-water bath. 9-BBN (0.5 M in THF, 16 mL, 1 eq.) was added, and the resulting mixture was stirred at room temperature for 4 hours. The mixture was cooled in an ice-water bath, and 10% aqueous NaOH (6.6 mL, degassed with Ar for 30 minutes) was added, followed by Cs₂CO₃ (5.33 g, 16.37 mmol, 2 equiv). Pd(OAc)₂ (184 mg, 0.82 mmol, 10 mol %), RuPhos (764 mg, 1.64 mmol, 20 mol %) and the Boc-protected indole from the previous step in 13.5 mL 1,4-dioxane was added in single portion and the mixture was heated to 90° C. and stirred overnight. The mixture was cooled to room temperature and diluted with H₂O and EtOAc. The layers were separated, the aqueous layer was extracted with EtOAc (3×25 mL), and the combined organic layers were dried with Na₂SO₄, filtered, and concentrated. The crude residue was purified by silica gel chromatography by eluting with a gradient of 5-20% EtOAc/hexanes to afford the title compound (2.1 g, 78% yield) as brown gum (the products contained some of the 9-BBN as insuperable mixture and was used in the next step without further purification.

¹H NMR (500 MHz, CDCl₃) δ 7.70 (d, J=2.3 Hz, 1H), 7.32 (d, J=8.5 Hz, 1H), 6.84 (d, J=10.9 Hz, 1H), 6.32 (s, 1H), 3.85 (s, 3H), 3.50-3.39 (m, 2H), 3.24-3.10 (m, 2H), 1.68 (s, 9H), 1.42 (s, 9H).

### Example 41. tert-butyl (2-(6-methoxy-1H-indol-2-yl)ethyl)carbamate

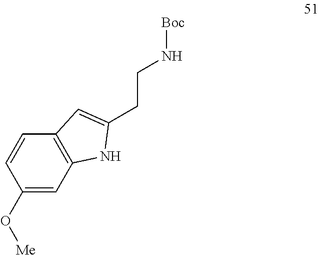 [SMILES: COc1ccc2cc(CCNC(=O)OC(C)(C)C)[nH]c2c1]

Compound 50 (800 mg, 3.0 mmol) was dissolved in 3 mL 1,4 dioxane and added to 60 mL of boiling water and the mixture was stirred at reflux for 12 hours (Wang, J. et al. 2009). After cooling to rt, the mixture was neutralized with a saturated solution of NaHCO₃ and extracted with EtOAc (×3). Compound 51 was isolated as a light brown oil (317 mg, 46% yield). ¹H NMR (500 MHz, D₂O) δ 8.22 (s, 1H), 7.40 (d, J=8.6 Hz, 1H), 6.81 (d, J=2.3 Hz, 1H), 6.19 (s, 1H), 3.83 (s, 3H), 3.45 (q, J=6.4 Hz, 2H), 2.91 (t, J=6.7 Hz, 2H), 1.44 (s, 9H). ¹³C NMR (101 MHz, CDCl₃) δ 156.15, 156.00, 136.87, 135.15, 122.98, 120.36, 109.41, 100.16, 94.67, 55.75, 29.10, 28.41.

### Example 42. tert-butyl (2-(6-methoxyindolin-2-yl)ethyl)carbamate 52

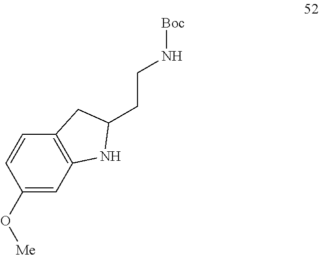 [SMILES: COc1ccc2c(c1)NC(CCNC(=O)OC(C)(C)C)C2]

A solution of 51 (619 mg, 2.13 mmol, 1 equiv) in 9 mL acetic acid was treated with a slow addition of NaBH₃CN at rt (536 mg, 8.53 mmol, 4 equiv) with some foaming and exothermic reaction being observed. The resulting mixture was stirred for additional 2 hours at that temperature, quenched with water (60 mL), basified with a saturated solution of NaHCO₃ and extracted three times with EtOAc (200 mL). The combined organic layers were dried with Na₂SO₄, filtered, and concentrated. The crude material was used directly for the next step.

### Example 43. tert-butyl (2-(1-(2-bromoethyl)-6-methoxyindolin-2-yl)ethyl)carbamate 53

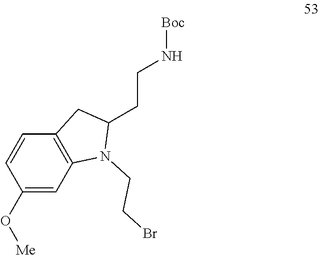 [SMILES: COc1ccc2c(c1)N(CCBr)C(CCNC(=O)OC(C)(C)C)C2]

A powdered potassium carbonate (833 mg, 6.39 mmol, 3 equiv) was added to the solution of the crude material 52 in 20 mL MeCN. The resulting suspension was treated with dibromoethane (3.7 mL, 42.6 mmol, 20 equiv) and the mixture was heated to reflux for 60 hours. The volatiles were removed under reduced pressure and the residue was diluted with water (70 mL) and extracted with DCM (50 mL×3). The combined organic layers were dried with Na₂SO₄, filtered, and concentrated. The crude residue was purified with silica gel chromatography by eluting with a gradient of 5-20% EtOAc/hexanes to afford the title compound (315 mg, 38% yield) as brown oil. 110 mg (20%) of the starting material was recovered. ¹H NMR (500 MHz, D₂O) δ 6.91 (d, J=7.9 Hz, 1H), 6.19 (dd, J=8.0, 2.3 Hz, 1H), 6.02 (d, J=2.3 Hz, 1H), 4.61 (s, 1H), 3.76 (s, 3H), 3.53-3.35 (m, 4H), 3.21 (q, J=6.9 Hz, 2H), 3.13-3.04 (m, 1H), 2.71-2.57 (m, 1H), 2.09-1.91 (m, 1H), 1.45 (s, 9H). 3C NMR (500 MHz, CDCl₃) δ 160.02, 155.77, 152.10, 124.31, 120.42, 101.44, 93.95, 79.05, 62.95, 55.22, 48.76, 36.98, 34.26, 33.78, 28.79, 28.25.

### Example 44. 8-methoxy-2,3,4,5,11,11a-hexahydro-1H-[1,4]diazepino[1,7-a]indole 54

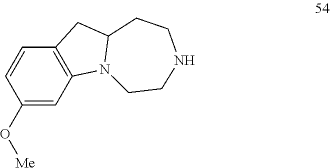 [SMILES: COc1ccc2c(c1)N1CCNCCC1C2]

A solution of 53 (315 mg, 0.79 mmol, 1 equiv) in 5 mL TFA was prepared at 0° C. After the mixture was stirred at that temperature for 1 hour, the TFA was removed in vacuum and the residue was dissolved in 26 mL of 20% aqueous 2-propanol containing K₂CO₃ (780 mg, 5.65 mmol, 7 equiv). The mixture was refluxed for 2 hours, cooled and concentrated under reduced pressure. The resulting crude mixture was partitioned between DCM and water. After the organic phase dried (Na₂SO₄), the solvent was evaporated and the crude residue was purified with silica gel chromatography by eluting with a 15% MeOH/83% EtOAc/2% TEA to afford the title compound (96 mg, 56% yield over 2 steps) as light yellow oil.

¹H NMR (500 MHz, CDCl₃) δ 6.90 (d, J=7.9 Hz, 1H), 6.17 (dd, J=7.9, 2.3 Hz, 1H), 5.95 (s, 1H), 3.99-3.87 (m, 1H), 3.63-3.53 (m, 1H), 3.27-3.06 (s, 3H), 2.98 (ddd, J=13.5, 10.2, 3.3 Hz, 1H), 2.67-2.56 (m, 1H), 2.11-2.00 (m, 1H), 1.98-1.88 (m, 1H). ¹³C NMR (500 MHz, CDCl₃) δ 123.90, 120.94, 100.77, 93.79, 64.57, 55.40, 49.84, 47.71, 47.50, 38.87, 36.25. LRMS (EI) calcd. For C₁₃H₁₈N₂O 218.1 [M]⁺, found: 218.1.

### Example 45. 8-methoxy-2,3,4,5-tetrahydro-1H-[1,4]diazepino[1,7-a]indole 55

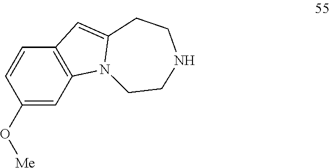 [SMILES: COc1ccc2cc3n(c2c1)CCNCC3]

A solution of 54 (95 mg, 0.44 mmol, 1 equiv) in 1,4 dioxane (2.5 mL) was treated with a single portion of DDQ (129 mg, 0.56 mmol, 1.3 equiv), instantly turning the mixture black. TLC analysis after 15 min showed unreacted starting material and the mixture was treated with additional 25 mol % DDQ for another 15 min. The reaction mixture was poured into 2N NaOH (13 mL) and extracted 3 times with DCM (10 mL). After the organic phase dried (Na₂SO₄), the solvent was evaporated and the crude residue was purified with silica gel chromatography by eluting with a 10% MeOH/88% EtOAc/2% TEA to afford the title compound (81 mg, 86% yield) as light brown solid. ¹H NMR (500 MHz, CDCl₃) δ 7.41 (d, J=9.2 Hz, 1H), 6.79-6.68 (m, 2H), 6.19 (s, 1H), 4.22-4.12 (m, 2H), 3.87 (s, 3H), 3.13-2.94 (m, 6H), 2.18 (s, 1H). ¹³C NMR (500 MHz, CDCl₃) δ 155.74, 141.11, 137.71, 122.21, 120.51, 108.65, 99.61, 93.02, 55.86, 50.05, 49.06, 48.03, 32.42. LRMS (EI) calcd. For C₁₃H₁₆N₂O 216.1 [M]⁺. found: 216.1.

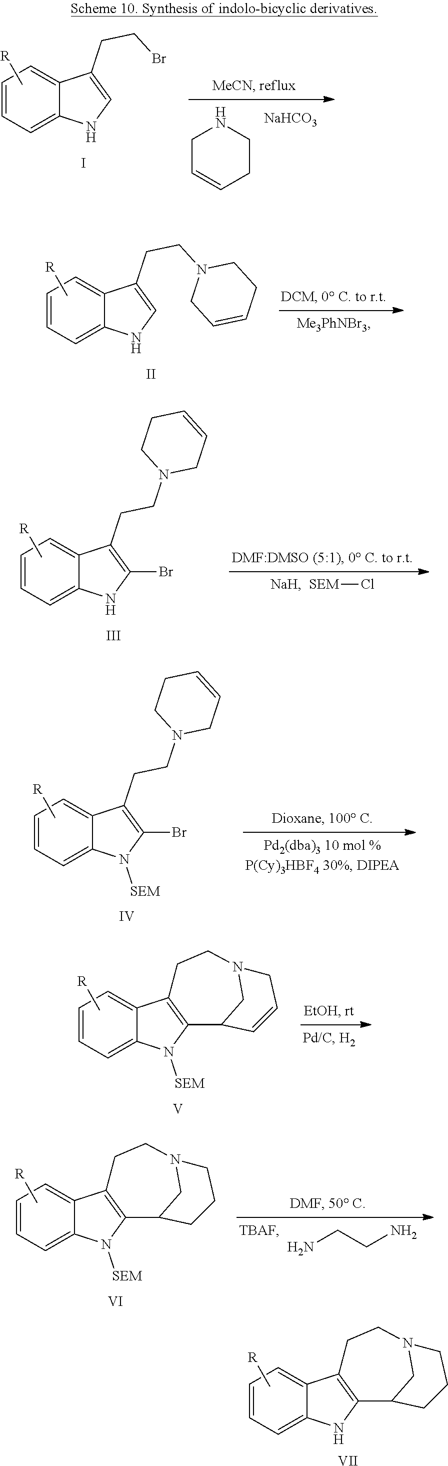

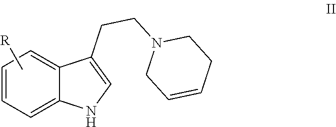

General procedure F: Substituted 3-(2-bromoethyl)-1H-indole I (1 equiv) and NaHCO₃ (4 equiv), were suspended in anhydrous CH₃CN (0.125 M). 1,2,3,6-Tetrahydropyridine (1.3 equiv) was added, and the resulting mixture was refluxed until TLC indicated the disappearance of the bromide (typically 1-2 days). The reaction was then diluted with H₂O, made strongly basic with aqueous NaOH, and extracted with CHCl₃ (3×). The combined organics were washed with H₂O, dried over Na₂SO₄, and concentrated to provide the crude product. The crude residue was purified with silica gel chromatography.

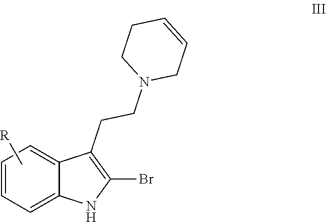

General procedure G: To a solution of derivative II (1 equiv) in anhydrous DCM (0.11 M) was added a solution of trimethylphenylammonium tribromide (0.9 equiv) in anhydrous DCM (0.18 M) dropwise at room temperature over 20 min. The resulting dark-red solution was stirred until TLC showed no starting material (˜10 min) and then quenched with H₂O and basified with saturated NH₄OH (the aqueous layer was removed. The remaining organic layer was then washed with H₂O and concentrated in vacuo to provide the crude bromide. The crude residue was purified with silica gel chromatography.

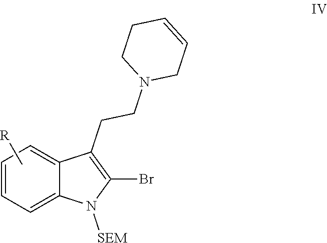

General procedure H: To an ice cold suspension of sodium hydride (60% dispersion in mineral oil, 1.6 equiv) and 5:1 DMF:DMSO (0.3 M) was added dropwise staring material III (1 equiv) dissolved in 1 mL DMF (0.3 M). The mixture was allowed to warm to rt and when hydrogen evolution had ceased (30-60 min), the mixture was cooled to 0° C. and 2-trimethylsilylethoxymethyl chloride (1.5 equiv) was added dropwise. The solution was then stirred at rt for 30 min before pouring into ice water. The aqueous layer was separated and extracted with ether (×3). The combined organics were washed with H₂O, dried over Na₂SO₄, and concentrated to provide the crude product. The crude residue was purified with silica gel chromatography.

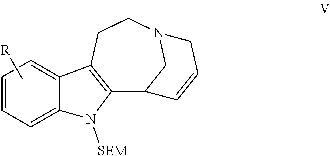

General procedure I: To a reaction tube under argon containing IV (1 equiv) followed by P(Cy)₃HBF₄ (30 mol) and Pd₂ (dba)₃ (15 mol) was added 1,4-Dioxane (0.1 M) followed by N,N-diisopropylethylamine (3 equiv). The mixture was heated to 100° C. for 6-12 hours with continuous stirring. The mixture was allowed to cool to ambient temperature, filtered over celite and concentrated in vacuo. The resulting material was subjected to flash chromatography.

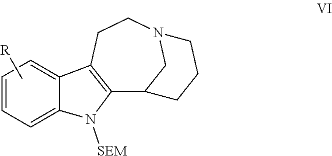

General procedure J: V (1 equiv) was dissolved in ethanol (0.01 M) and 10% palladium on carbon (1 mg per mg of starting material) was added. The reaction mixture was kept under hydrogen atmosphere (40 psi) and was stirred at room temperature for 12 hours. Obtained mixture was filtered through celite and concentrated under reduced pressure. The resulting material was subjected to flash chromatography

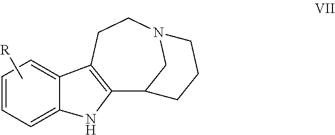

General procedure K: A mixture of VI (1 equiv), tetra n-butylammonium fluoride (1 M solution, 3 equiv), DMF (0.9 M), and ethylenediamine (6 equiv) was stirred at 45° C. for 24 h. At this time, if TLC analysis showed reminding SM a additional 3 equiv of 1 M TBAF solution was added and the mixture stirred for 12 h longer. The reaction mixture was poured into water and extracted with ether. The extract was washed successively with dilute hydrochloric acid and 10% sodium bicarbonate solution, and after the extract was dried, the ether was removed in vacuo. The resulting material was subjected to flash chromatography.

### Example 46. 3-(2-(3,6-dihydropyridin-1 (2H)-yl)ethyl)-1H-indole 56

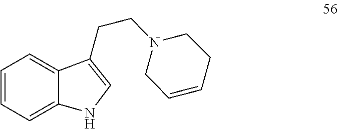 [SMILES: C1=CCN(CCc2c[nH]c3ccccc23)CC1]

General procedure F was applied using 3-(2-bromoethyl)-1H-indole (4.0 g, 17.85 mmol), NaHCO₃ (6.0 g, 71.40 mmol, 4 equiv), 140 mL anhydrous CH₃CN, and 1,2,3,6-tetrahydropyridine (2.1 mL, 23.20 mmol, 1.3 equiv. The crude residue was purified by silica gel chromatography, eluting with a 5% MeOH/93% EtOAc/2% TEA to afford the title compound (3 g, 74% yield) as a light brown solid. ¹H NMR (300 MHz, CDCl₃) δ 8.15 (s, 1H), 7.64 (d, J=7.8 Hz, 1H), 7.37-7.30 (m, 1H), 7.23-7.07 (m, 2H), 7.02 (s, 1H), 5.86-5.66 (m, 2H), 3.16-3.10 (m, 2H), 3.08-3.00 (m, 2H), 2.84-2.76 (m, 2H), 2.71 (t, J=5.7 Hz, 2H), 2.30-2.21 (m, 2H). ¹³C NMR (300 MHz, CDCl₃) δ 136.31, 127.56, 125.39, 125.31, 121.93, 121.50, 119.20, 118.86, 114.53, 111.13, 59.35, 52.76, 50.19, 26.29, 23.16.

### Example 47. 2-bromo-3-(2-(3,6-dihydropyridin-1 (2H)-yl)ethyl)-1H-indole 57

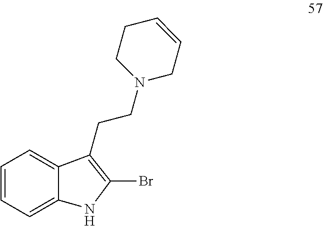 [SMILES: Brc1[nH]c2ccccc2c1CCN1CC=CCC1]

General procedure G was applied using 56 (3.0 g, 13.26 mmol, 1 equiv) in anhydrous DCM (120 mL, 0.11 M), and trimethylphenylammonium tribromide (4.48 g, 11.93 mmol. 0.9 equiv) in anhydrous DCM (65 mL, 0.18 M). The crude residue was purified with silica gel chromatography by eluting with a 2% MeOH/96% EtOAc/2% TEA to afford the title compound (1.79 g, 44% yield) as brown solid. ¹H NMR (400 MHz, CDCl₃) 8.28 (s, 1H), 7.54 (d, J=8.6 Hz, 1H), 7.29-7.22 (m, 1H), 7.19-7.05 (m, 2H), 5.84-5.67 (m, 2H), 3.18-3.09 (m, 2H), 3.01-2.93 (m, 2H), 2.75-2.66 (m, 4H), 2.31-2.20 (m, 2H). ¹³C NMR (400 MHz, CDCl₃) δ 136.19, 127.73, 125.32, 125.25, 122.22, 119.96, 118.18, 113.71, 110.47, 108.18, 58.15, 52.61, 50.04, 26.19, 22.80. LRMS (EI) calcd. For C₁₅H₁₇BrN₂ 304.1 [M]⁺. found: 304.2.

### Example 48. 2-bromo-3-(2-(3,6-dihydropyridin-1(2H)-yl)ethyl)-1-((2-(trimethylsilyl)ethoxy)methyl)-1H-indole 58

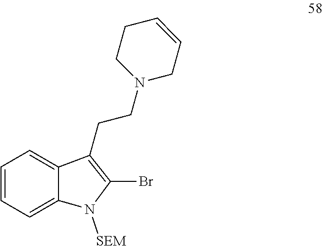 [SMILES: C[Si](C)(C)CCOCn1c(Br)c(CCN2CC=CCC2)c2ccccc21]

General procedure H was applied using sodium hydride (21.0 mg of 60% dispersion in mineral oil, 0.52 mmol, 1.6 equiv) in 5:1 DMF:DMSO (0.8:0.2 mL), 57 (100 mg, 0.33 mmol, 1 equiv) dissolved in 1 mL DMF. and 2-trimethylsilylethoxymethyl chloride (86.0 uL, 0.49 mmol, 1.5 equiv). The crude residue was purified by silica gel chromatography, eluting with a 1:1 hexanes:EtOAc 2% TEA to afford the title compound (79 mg, 55% yield) as light brown solid. ¹H NMR (500 MHz, CDCl₃) δ 7.57 (d, J=7.8 Hz, 1H), 7.44 (d, J=8.2 Hz, 1H), 7.25-7.19 (m, 1H), 7.17-7.12 (m, 1H), 5.82-5.77 (m, 1H), 5.75-5.70 (m, 2H), 5.55 (s, 2H), 3.59-3.50 (m, 2H), 3.17-3.11 (m, 2H), 3.03-2.98 (m, 2H), 2.74-2.62 (m, 4fH), 2.29-2.21 (m, 2H), 0.93-0.82 (m, 2H), -0.06 (s, 9H).

### Example 49. 8-((2-(trimethylsilyl)ethoxy)methyl)-1,4,7,8-tetrahydro-2H-3,7-methanoazonino[5,4-b]indole 59

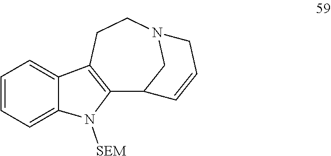 [SMILES: C[Si](C)(C)CCOCn1c2c(c3ccccc31)CCN1CC=CC2C1]

General procedure I was applied using 58 (920 mg, 2.11 mmol. 1 equiv), P(Cy)₃HBF₄ (232 mg, 0.63 mmol, 30 mol %) and Pd₂ (dba)₃ (290 mg, 0.32 mmol, 15 mol %) and N,N-diisopropylethylamine (1.1 mL, 6.34 mmol, 3 equiv) in 1,4-Dioxane (20.0 mL). The resulting material was subjected to flash chromatography (3:7 EtOAc/hexanes, 2% TEA) to afford the title compound (166 mg, 22% yield) as light brown gum. ¹H NMR (500 MHz, CDCl₃) δ 7.47 (d, J=6.7 Hz, 1H), 7.38 (d, J=8.0 Hz, 1H), 7.20-7.10 (m, 2H), 5.90-5.84 (m, 2H), 5.51 (s, 2H), 3.88-3.79 (m, 1H), 3.59-3.44 (m, 5H), 3.38-3.29 (m, 2H), 3.24 (ddd, J=13.7, 11.6, 2.1 Hz, 1H), 3.12 (ddd, J=15.7, 11.6, 3.0 Hz, 1H), 2.79 (ddd, J=15.7, 4.4, 2.1 Hz, 1H), 0.98-0.84 (m, 2H), -0.02 (s, 9H. ¹³C NMR (500 MHz, CDCl₃) δ 139.27, 136.26, 128.17, 126.15, 125.38, 121.21, 119.75, 117.66, 114.48, 109.12, 71.92, 65.53, 56.12, 51.34, 49.55, 31.38, 23.09, 17.97, -1.34, -1.36. LRMS (EI) calcd. For C₂₁H₃₀N₂OSi 354.2 [M]⁺. found: 354.2.

### Example 50. 8-((2-(trimethylsilyl)ethoxy)methyl)-1,4,5,6,7,8-hexahydro-2H-3,7-methanoazonino[5,4-b]indole 60

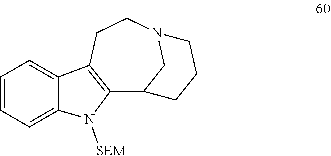 [SMILES: C[Si](C)(C)CCOCn1c2c(c3ccccc31)CCN1CCCC2C1]

General procedure J was applied using 59 (166 mg, 0.47 mmol) dissolved in ethanol (40 mL) and palladium on carbon (10%, 166 mg). The resulting material was subjected to flash chromatography (1:1 EtOAc/hexanes, 2% TEA) to afford the title compound (90 mg, 53% yield) as light brown solid. ¹H NMR (400 MHz, CDCl₃) δ 7.48 (d, J=6.7 Hz, 1H), 7.37 (d, J=0.9 Hz, 1H), 7.22-7.16 (m, 1H), 7.15-7.08 (m, 1H), 5.50-5.36 (m, 2H), 3.59-3.48 (m, 2H), 3.44-3.40 (m, 1H), 3.40-3.36 (m, 2H), 3.31-3.23 (m, 2H), 3.21-3.16 (m, 3H), 2.99-2.92 (m, 1H), 1.98-1.91 (m, 1H), 1.82-1.68 (m, 1H), 1.34-1.29 (m, 1H), -0.04 (s, 9H). 13C NMR (400 MHz, CDCl₃) δ 140.99, 136.93, 128.68, 121.51, 119.56, 117.98, 113.51, 108.87, 71.92, 65.59, 55.47, 55.05, 50.04, 32.29, 30.69, 26.92, 21.26, 17.96, −1.40. LRMS (EI) calcd. For C₂₁H₃₂N₂OSi 356.2 [M]⁺. found: 356.2.

### Example 51. 1,4,5,6,7,8-hexahydro-2H-3,7-methanoazonino[5,4-b]indole 61

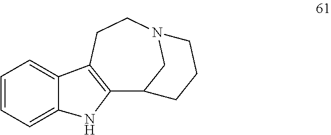 [SMILES: c1ccc2c3c([nH]c2c1)C1CCCN(CC3)C1]

General procedure K was applied using 60 (90 mg, 0.25 mmol), tetra n-butylammonium fluoride (0.8 mL 1 M, 0.8 mmol, 3 equiv), DMF (0.3 mL, 0.9 M), and ethylenediamine (0.1 mL, 1.50 mmol, 6 equiv). After 24 h additional 0.4 mL 1 M TBAF solution was added and the mixture stirred for 12 h longer. The resulting material was subjected to flash chromatography (8:2 EtOAc/hexanes, 2% TEA) to afford the title compound (41 mg, 72% yield) as light brown solid. ¹H NMR (400 MHz, MeOD) δ 7.38 (d, J=8.8 Hz, 1H), 7.23 (d, J=7.9 Hz, 1H), 7.05-6.93 (m, 2H), 3.36-3.32 (m, 1H), 3.28-3.25 (m, 2H), 3.23-3.15 (m, 2H), 3.10-3.04 (m, 2H), 3.00-2.91 (m, 2H), 2.01-1.95 (m, 1H), 1.95-1.87 (m, 1H), 1.83-1.69 (m, 1H), 1.34-1.26 (m, 1H). ¹³C NMR (400 MHz, MeOD) δ 139.25, 135.56, 129.04, 120.29, 118.06, 116.96, 110.38, 109.91, 54.98, 54.21, 49.34, 34.70, 30.44, 25.33, 20.23. LRMS (EI) calcd. For C₁₅H₁₅N₂ 226.1 [M]⁺. found: 226.1.

### Example 52. 3-(2-(3,6-dihydropyridin-1 (2H)-yl)ethyl)-5-methoxy-1H-indole 31

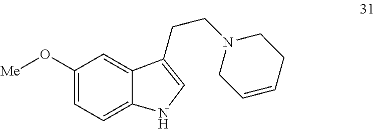 [SMILES: COc1ccc2[nH]cc(CCN3CC=CCC3)c2c1]

General procedure F was applied using 3-(2-bromoethyl)-5-methoxy-1H-indole, (254 mg, 1.97 mmol, 1 equiv), NaHCO₃ (336 mg, 1.00 mmol, 4 equiv), 8 mL anhydrous CH₃CN, and 1,2,3,6-tetrahydropyridine (0.12 mL, 1.30 mmol, 1.3 equiv). The crude residue was purified with silica gel chromatography by eluting with a 5% MeOH/93% EtOAc/2% TEA to afford the title compound (203 mg, 74% yield) as light brown solid. ¹H NMR (500 MHz, CDCl₃) δ 8.00 (s, 1H), 7.28 (d, J=5.3 Hz, 1H), 7.10 (d, J=2.5 Hz, 1H), 7.04 (d, J=2.4 Hz, 1H), 6.88 (dd, J=8.7, 2.5 Hz, 1H), 5.85-5.79 (m, 1H), 5.77-5.72 (m, 1H), 3.89 (s, 3H), 3.19-3.13 (m, 2H), 3.06-3.00 (m, 2H), 2.84-2.78 (m, 2H), 2.74 (t, J=5.7 Hz, 2H), 2.32-2.26 (m, 2H). ¹³C NMR (500 MHz, CDCl₃) δ 153.93, 131.41, 127.87, 125.33, 125.05, 122.36, 112.15, 111.84, 100.74, 59.11, 55.99, 52.64, 50.12, 26.07, 23.12. LRMS (EI) calcd. For C₁₆H₂₀N₂O 256.1 [M]⁺. found: 256.0.

### Example 53. 2-bromo-3-(2-(3,6-dihydropyridin-1(2H)-yl)ethyl)-5-methoxy-1H-indole 62

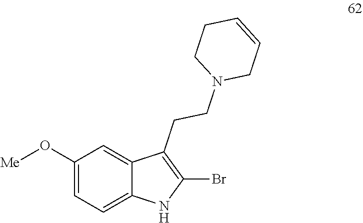 [SMILES: COc1ccc2[nH]c(Br)c(CCN3CC=CCC3)c2c1]

General procedure G was applied using 31 (10.0 g, 39.01 mmol, 1 equiv) in anhydrous DCM (350 mL, 0.11 M), and trimethylphenylammonium tribromide (13.2 g, 35.11 mmol. 0.9 equiv) in anhydrous DCM (200 mL, 0.18 M). The crude residue was purified with silica gel chromatography by eluting with a 2% MeOH/96% EtOAc/2% TEA to afford the title compound (2.0 g, 15% yield) as light yellow solid. *The reaction was quenched immediately after the dropwise addition of the bromination reagent to avoid additional bromination od the a position of the methoxy, 29% of the starting material were recovered. ¹H NMR (500 MHz, CDCl₃) δ 8.58 (s, 1H), 7.12 (d, J=8.8 Hz, 1H), 6.97 (d, J=2.4 Hz, 1H), 6.79 (dd, J=8.8, 2.4 Hz, 1H), 5.84-5.68 (m, 2H), 3.82 (s, 3H), 3.19-3.14 (m, 2H), 2.97-2.91 (m, 2H), 2.75-2.66 (m, 4H), 2.30-2.23 (m, 2H). 13C NMR (500 MHz, CDCl₃) δ 173.16, 154.85, 146.91, 139.33, 125.37, 124.52, 116.35, 113.03, 109.60, 84.72, 55.81, 53.58, 52.71, 49.90, 33.34, 25.82. LRMS (EI) calcd. For C₁₆H₁₉BrN₂O 334.2 [M]⁺, found: 334.2.

### Example 54. 2-bromo-3-(2-(3,6-dihydropyridin-1(2H)-yl)ethyl)-5-methoxy-1-((2-(trimethylsilyl)ethoxy)methyl)-1H-indole 63

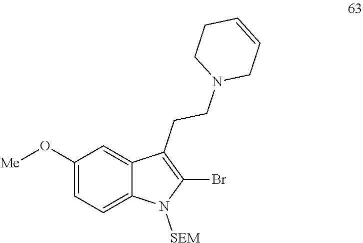 [SMILES: COc1ccc2c(c1)c(CCN1CC=CCC1)c(Br)n2COCC[Si](C)(C)C]

General procedure H was applied using sodium hydride (21.0 mg of 60% dispersion in mineral oil, 0.52 mmol, 1.6 equiv) in 5:1 DMF:DMSO (0.8:0.2 mL), 62 (100 mg, 0.33 mmol, 1 equiv) dissolved in 1 mL DMF. and 2-trimethylsilylethoxymethyl chloride (86.0 uL, 0.49 mmol, 1.5 equiv). The crude residue was purified with silica gel chromatography by eluting with a 1:1 hexanes:EtOAc 2% TEA to afford the title compound (79 mg, 55% yield) as light brown solid. ¹H NMR (500 MHz, CDCl₃) δ 7.33 (d, J=8.8 Hz, 1H), 7.01 (d, J=2.5 Hz, 1H), 6.86 (dd, J=8.9, 2.4 Hz, 1H), 5.82-5.76 (m, 1H), 5.74-5.69 (m, 1H), 5.50 (s, 2H), 3.85 (s, 3H), 3.55-3.46 (m, 2H), 3.15-3.07 (m, 2H), 3.01-2.94 (m, 2H), 2.73-2.59 (m, 4H), 2.29-2.19 (m, 2H), 0.91-0.82 (m, 2H), −0.07 (s, 9H). ¹³C NMR (500 MHz, CDCl₃) δ 154.63, 132.20, 128.17, 125.30, 113.89, 112.83, 112.01, 110.89, 100.50, 73.56, 65.74, 57.99, 55.91, 52.72, 50.06, 26.29, 23.37, 17.79, −1.28, −1.42. LRMS (EI) calcd. For C₂₂H₃₃BrN₂O₂Si 464.2 [M]⁺. found: 464.1.

### Example 55. 11-methoxy-8-((2-(trimethylsilyl)ethoxy)methyl)-1,4,7,8-tetrahydro-2H-3,7-methanoazonino[5,4-b]indole 64

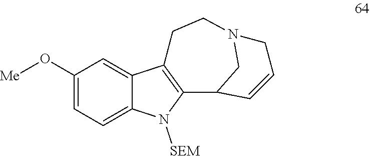 [SMILES: COc1ccc2c(c1)c1c(n2COCC[Si](C)(C)C)C2C=CCN(CC1)C2]

General procedure I was applied using 63 (1.95 g, 4.48 mmol. 1 equiv), P(Cy)₃HBF₄ (493 mg, 1.34 mmol, 30 mol %) and Pd₂ (dba)₃ (672 mg, 0.67 mmol, 15 mol %) and N,N-diisopropylethylamine (2.35 mL, 13.43 mmol, 3 equiv) in 1,4-Dioxane (45.0 mL). The resulting material was subjected to flash chromatography (3:7 EtOAc/hexanes, 2% TEA) to afford the title compound (351 mg, 22% yield) as brown gum. ¹H NMR (500 MHz, CDCl₃) δ 7.28 (s, 1H), 6.92 (d, J=2.4 Hz, 1H), 6.83 (dd, J=8.8, 2.4 Hz, 1H), 5.87 (s, 2H), 3.87 (s, 3H), 3.54 (dd, J=13.8, 4.5 Hz, 1H), 3.50-3.42 (m, 4H), 3.38-3.29 (m, 2H), 3.24 (ddd, J=13.7, 11.6, 2.2 Hz, 1H), 3.10 (ddd, J=14.9, 11.6, 2.9 Hz, 1H), 2.78-2.71 (m, 1H), 1.89-1.65 (m, 1H), 0.97-0.83 (m, 2H), -0.02 (s, 9H). ¹³C NMR (500 MHz, CDCl₃) δ 154.37, 139.94, 131.43, 128.46, 126.13, 125.33, 114.14, 110.91, 109.85, 99.95, 72.04, 65.46, 56.12, 55.95, 51.29, 49.50, 31.41, 23.14, 17.94, -1.38. LRMS (EI) calcd. For C₂₂H₃₂N₂O₂Si 384.2 [M]⁺. found: 384.2.

### Example 56. 11-methoxy-8-((2-(trimethylsilyl) ethoxy)methyl)-1,4,5,6,7,8-hexahydro-2H-3,7-methanoazonino[5,4-b]indole 65

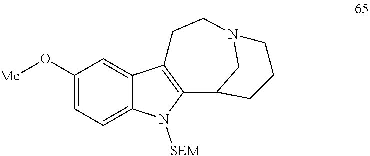 [SMILES: COc1ccc2c(c1)c1c(n2COCC[Si](C)(C)C)C2CCCN(CC1)C2]

General procedure J was applied using 64 (350 mg, 1.38 mmol) and palladium on carbon (10%, 350 mg) dissolved in ethanol (60 mL). The resulting material was subjected to flash chromatography (1:1 EtOAc/hexanes, 2% TEA) to afford the title compound (201 mg, 42% yield) as light brown solid. ¹H NMR (400 MHz, CDCl₃) δ 7.25 (d, J=9.0 Hz, 1H), 6.93 (d, J=2.4 Hz, 1H), 6.84 (dd, J=8.8, 2.5 Hz, 1H), 5.43-5.30 (m, 2H), 3.86 (s, 3H), 3.56-3.47 (m, 2H), 3.42-3.35 (m, 2H), 3.33-3.24 (m, 1H), 3.22-3.12 (m, 3H), 2.95-2.86 (m, 1H), 1.92 (t, J=4.1 Hz, 1H), 1.89-1.82 (m, 1H), 1.78-1.71 (m, 1H), 1.50-1.38 (m, 1H), 1.33-1.22 (m, 2H), 0.92-0.85 (m, 2H), -0.05 (s, 9H). ¹³C NMR (400 MHz, CDCl₃) δ 154.31, 141.65, 132.09, 128.96, 113.12, 111.09, 109.58, 100.54, 72.06, 65.52, 56.02, 55.38, 55.04, 50.03, 32.30, 30.64, 26.91, 21.20, 17.94, −1.40. LRMS (EI) calcd. For C₂₂H₃₄N₂O₂Si 386.2 [M]⁺. found: 386.2.

### Example 57. 11-methoxy-1,4,5,6,7,8-hexahydro-2H-3,7-methanoazonino[5,4-b]indole 32

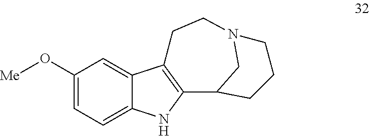 [SMILES: COc1ccc2[nH]c3c(c2c1)CCN1CCCC3C1]

General procedure K was applied using 65 (200 mg, 0.517 mmol), tetra n-butylammonium fluoride (1.55 mL 1 M, 1.55 mmol, 3 equiv), DMF (0.6 mL, 0.9 M), and ethylenediamine (0.2 mL, 3.10 mmol, 6 equiv). After 24 h additional 1.0 mL 1 M TBAF solution was added and the mixture stirred for 12 h longer. The resulting material was subjected to flash chromatography (8:2 EtOAc/hexanes, 2% TEA) to afford the title compound (64.0 mg, 48% yield) as light brown solid. ¹H NMR (500 MHz, CDCl₃) δ 7.88 (s, 1H), 7.16 (d, J=8.7 Hz, 1H), 6.95 (d, J=2.4 Hz, 1H), 6.80 (dd, J=8.7, 2.5 Hz, 1H), 3.87 (s, 3H), 3.45-3.39 (m, 1H), 3.39-3.33 (m, 2H), 3.30 (dd, J=12.7, 3.5 Hz, 1H), 3.25 (dd, J=14.3, 3.8 Hz, 1H), 3.21-3.15 (m, 2H), 2.88 (dt, J=16.1, 3.4 Hz, 1H), 2.83 (p, J=2.9 Hz, 1H), 2.03-1.94 (m, 1H), 1.92 (ddt, J=12.9, 8.2, 4.1 Hz, 1H), 1.84-1.72 (m, 1H), 1.34-1.28 (m, 1H). ¹³C NMR (500 MHz, CDCl₃) δ 153.99, 140.84, 130.12, 129.87, 111.80, 110.96, 100.33, 56.07, 55.26, 49.97, 36.05, 31.40, 26.78, 21.25. LRMS (EI) calcd. For C₆H₂₀N₂O 256.2 [M]⁺. found: 256.1.

### Example 58. 1,4,5,6,7,8-hexahydro-2H-3,7-methanoazonino[5,4-b]indol-11-ol 66

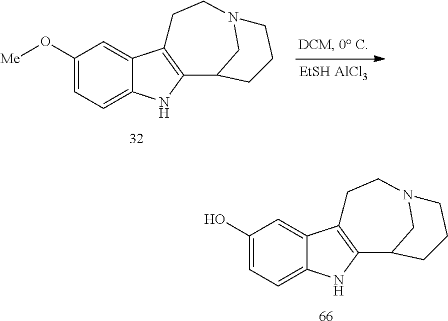

To a solution of 32 (8.0 mg, 0.031 mmol, 1 equiv.) in dry dichloromethane (0.25 mL, 0.125 M) at 0° C. was added aluminum chloride (24.87 mg, 0.186 mmol, 6 equiv.) followed by ethanethiol (0.042 mL, 0.559 mmol, 18 equiv.), and the resulting mixture was allowed to warm to room temperature and stirred until TLC indicated the complete consumption of starting material (typically <1.5 h). The reaction was then quenched with saturated aqueous NaHCO₃ (100 mL per mmol of starting material) and extracted with DCM (4×-6×, until no further extraction by TLC). The combined organic layers were dried over Na₂SO₄ and concentrated to provide the crude product. The resulting material was subjected to flash chromatography (9:1 DCM/MeOH, 1% sat. sol. NH₄OH) to afford the title compound (3.7.0 mg, 49% yield) as light off white solid. ¹H NMR (500 MHz, MeOD) δ 7.12 (d, J=8.5 Hz, 1H), 6.83 (d, J=2.3 Hz, 1H), 6.66 (d, J=6.0 Hz, 1H), 3.81-3.72 (m, 1H), 3.67-3.59 (m, 3H), 3.50-3.45 (m, 1H), 3.44-3.39 (m, 2H), 3.19 (t, J=6.0 Hz, 2H), 2.07-1.99 (m, 2H), 1.95-1.82 (m, 1H), 1.74-1.67 (m, 1H). ¹³C NMR (500 MHz, MeOD) δ 150.20, 136.27, 130.57, 128.56, 111.19, 110.89, 108.93, 101.52, 55.61, 51.75, 50.39, 31.21, 27.28, 20.37, 16.51. LRMS (EI) calcd. For C₁₅H₁₈N₂O 242.1 [M]⁺. found: 242.1.

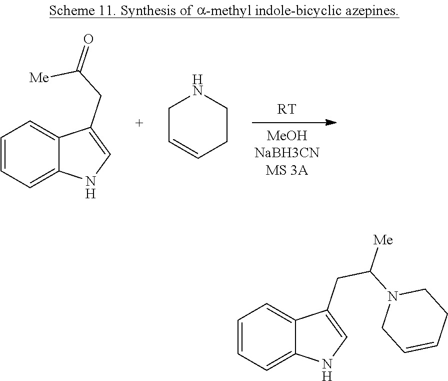

### Example 59. 3-(2-(3,6-dihydropyridin-1 (2H)-yl)propyl)-1H-indole 67

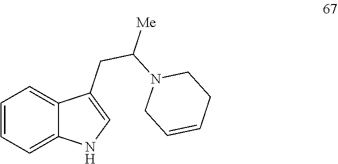 [SMILES: CC(Cc1c[nH]c2ccccc12)N1CC=CCC1]

Indole-3-acetone (346 mg. 2.0 mmol) and 1,2,3,6-tetrahydropyridine (249 mg, 3.0 mmol) were combined in MeOH (8 mL), NaBH₃CN (252 mg, 4.0 mmol) and 3 Å molecular sieves (200 mg) were added and the reaction was stirred at room temperature 90 h. Mixture was diluted with H₂O (25 mL) and extracted with AcOEt (4×10 mL), extracts were dried over Na₂SO₄, filtered and concentrated. Crude material was purified by column chromatography, 50% AcOEt in Hex, 50% AcOEt in Hex+2% Et₃N to AcOEt+2% Et₃N. Product was obtained as a brown amorphous solid (273 mg, 57%).

¹H NMR (500 MHz, CDCl₃) δ 8.14-7.97 (m, 1H), 7.63 (dd, J=7.9, 1.1 Hz, 1H), 7.36 (d, J=8.2 Hz, 1H), 7.19 (ddd, J=8.1, 7.0, 1.2 Hz, 1H), 7.12 (ddd, J=8.0, 6.9, 1.1 Hz, 1H), 7.03 (d, J=2.2 Hz, 1H), 5.83-5.73 (m, 2H), 3.26 (dq, J=5.1, 2.8 Hz, 2H), 3.19 (dd, J=13.9, 3.7 Hz, 1H), 3.05 (dqd, J=10.2, 6.5, 3.6 Hz, 1H), 2.85-2.73 (m, 2H), 2.67 (dd, J=13.9, 10.1 Hz, 1H), 2.25 (dd, J=5.6, 3.5 Hz, 2H), 1.04 (d, J=6.5 Hz, 3H). ¹³C NMR (126 MHz, CDCl₃) δ 136.4, 127.9, 126.2, 125.5, 122.3, 122.0, 119.3, 119.1, 114.7, 111.2, 59.9, 48.3, 45.6, 28.7, 27.1, 15.0. LRMS (ESI⁺) calcd. for C₁₆H₂₁N₂[M+H]⁺ 241.2, found 241.1.

### Example 60. 2-bromo-3-(2-(3,6-dihydropyridin-1(2H)-yl)propyl)-1-((2-(trimethylsilyl)ethoxy)methyl)-1H-indole 68

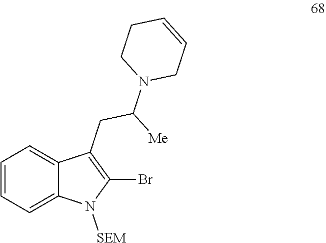 [SMILES: CC(Cc1c(Br)n(COCC[Si](C)(C)C)c2ccccc12)N1CC=CCC1]

General procedure G was applied using 67 (273 mg, 1.14 mmol, 1 equiv) in anhydrous DCM (10.3 mL), and trimethylphenylammonium tribromide (470 mg, 1.25 mmol. 1.1 equiv) in anhydrous DCM (5.7 mL). The crude residue was used for the next step without purification. Crude bromide was protected with SEM group using general procedure H. Crude material was purified by column chromatography using 10% AcOEt in Hex+2% Et₃N. Product was obtained as a brown oil (75 mg, 19% over two steps).

¹H NMR (500 MHz, CDCl₃) δ 7.58 (d, J=7.8 Hz, 1H), 7.47 (d, J=8.1 Hz, 1H), 7.24 (t, J=7.7 Hz, 1H), 7.17 (t, J=7.5 Hz, 1H), 5.85-5.75 (m, 2H), 5.59 (s, 2H), 3.58-3.50 (m, 2H), 3.31 (q, J=15.7 Hz, 2H), 3.18-3.06 (m, 2H), 2.90-2.78 (m, 2H), 2.73 (dd, J=13.4, 10.4 Hz, 1H), 2.27 (s, 2H), 1.03 (d, J=6.5 Hz, 2H), 0.95-0.85 (m, 3H), −0.05 (s, 9H). ¹³C NMR (126 MHz, CDCl₃) δ 137.1, 128.2, 126.1, 125.5, 122.4, 120.4, 118.6, 114.7, 113.3, 110.1, 73.5, 65.8, 59.5, 48.4, 45.5, 28.1, 27.1, 17.9, 15.3, −1.4. LRMS (ESI⁺) calcd. for C₂₂H₃₄BrN₂OSi [M+H]⁺ 449.2. found 449.1.

### Example 61. (2R)-2-methyl-8-((2-(trimethylsilyl) ethoxy)methyl)-1,4,5,6,7,8-hexahydro-2H-3,7-methanoazonino[5,4-b]indole 69 and (2S)-2-methyl-8-((2-(trimethylsilyl)ethoxy)methyl)-1,4,5,6,7,8-hexahydro-2H-3,7-methanoazonino[5,4-b]indole 70

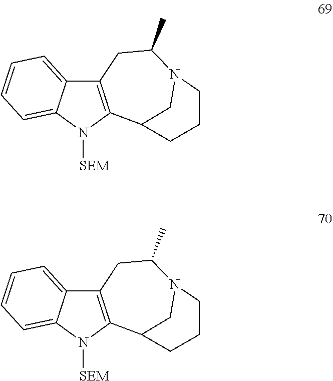 [SMILES: C[C@@H]1Cc2c(n(COCC[Si](C)(C)C)c3ccccc23)C2CCCN1C2 ; C[C@H]1Cc2c(n(COCC[Si](C)(C)C)c3ccccc23)C2CCCN1C2]

General procedure I was applied using 68 (75 mg, 0.17 mmol). The crude product was purified by two preparative TLC, 1) 95:5:0.5 DCM:MeOH:NH₄OH and 2) 50% AcOEt in Hex+2% Et₃N. Slightly impure material was reacted according to general procedure J and the crude material was purified by preparative TLC, 1) 95:5:0.5 DCM:MeOH:NH₄OH and 2) 50% AcOEt in Hex+2% Et₃N.

Compound 69

¹H NMR (500 MHz, CDCl₃) δ 7.50 (s, 1H), 7.40 (d, J=8.2 Hz, 1H), 7.23 (d, J=7.9 Hz, 1H), 7.16 (t, J=7.4 Hz, 1H), 5.50-5.40 (m, 2H), 3.70-3.59 (m, 1H), 3.59-3.48 (m, 1H), 3.48-3.37 (m, 1H), 3.36-3.27 (m, 1H), 3.18-3.08 (m, 1H), 3.08-2.94 (m, 2H), 1.99 (d, J=8.9 Hz, 2H), 1.94-1.62 (m, 2H), 1.53-1.42 (m, 2H), 1.37-1.30 (m, 2H), 1.30-1.24 (m, 1H), 1.23-1.20 (m, 1H), 0.94-0.87 (m, 2H), -0.03 (s, 9H). LRMS (ESI⁺) calcd. for C₂₂H₃₄N₂OSi [M+H]⁺ 371.3, found 371.4.

Compound 70

¹H NMR (500 MHz, CDCl₃) δ 7.50 (d, J=7.7 Hz, 1H), 7.41 (d, J=8.1 Hz, 1H), 7.24 (t, J=7.7 Hz, 1H), 7.17 (t, J=7.4 Hz, 1H), 5.50-5.38 (m, 2H), 3.86 (d, J=14.1 Hz, 1H), 3.59-3.46 (m, 2H), 3.46-2.89 (m, 4H), 1.96 (t, J=13.3 Hz, 1H), 1.90-1.77 (m, 1H), 1.76-1.51 (m, 4H), 1.51-1.19 (m, 4H), 0.96-0.82 (m, 2H), −0.03 (d, J=3.7 Hz, 9H). LRMS (ESI⁺) calcd. for C₂₂H₃₄N₂OSi [M+H]⁺ 371.3. found 371.4.

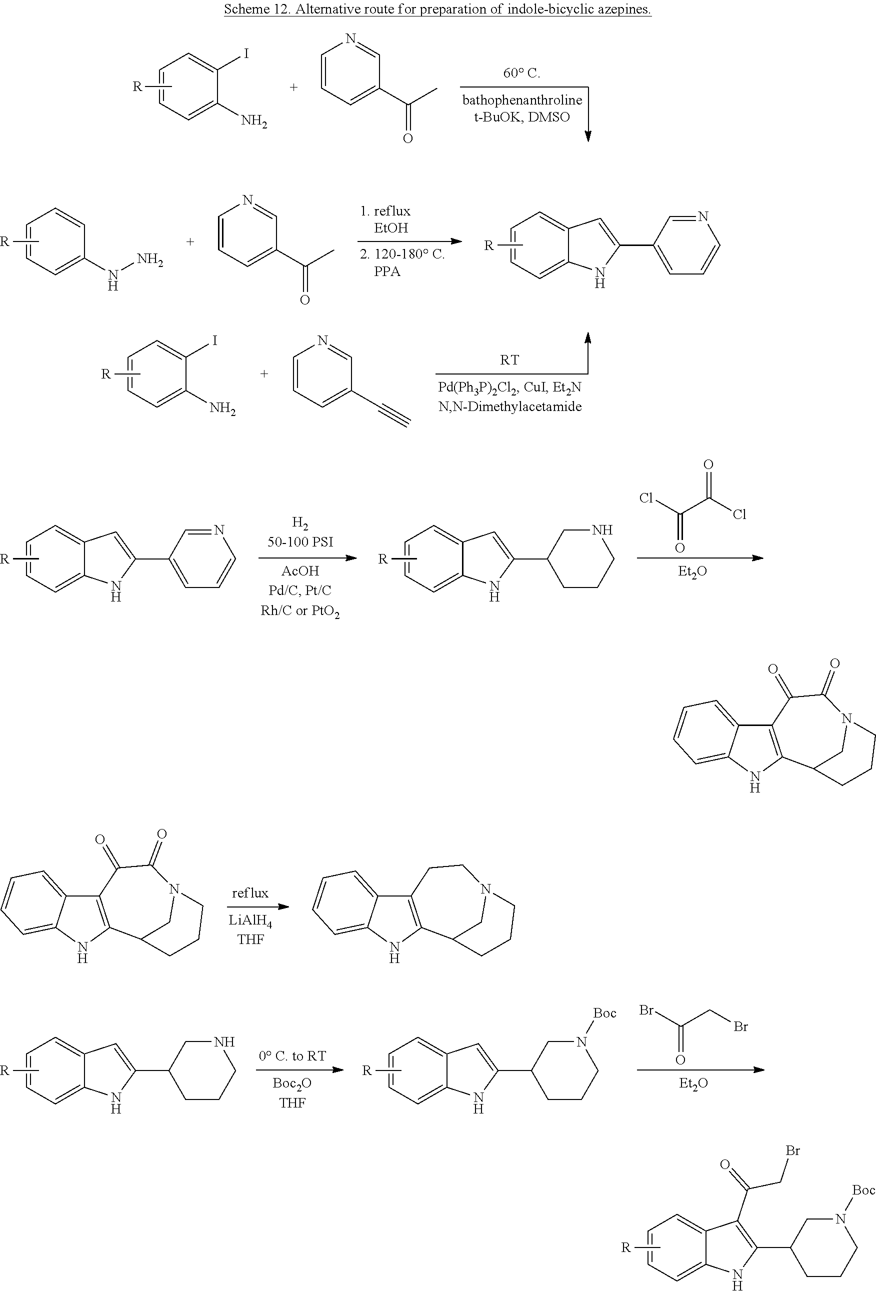

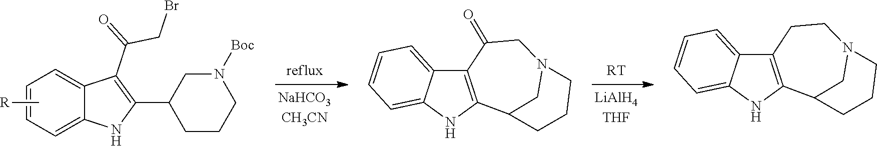

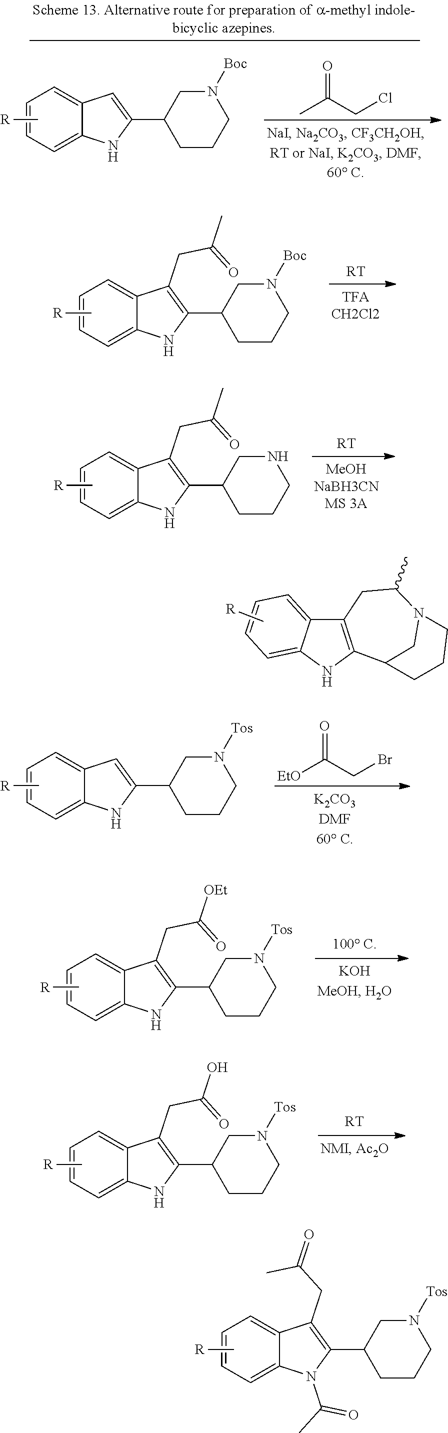

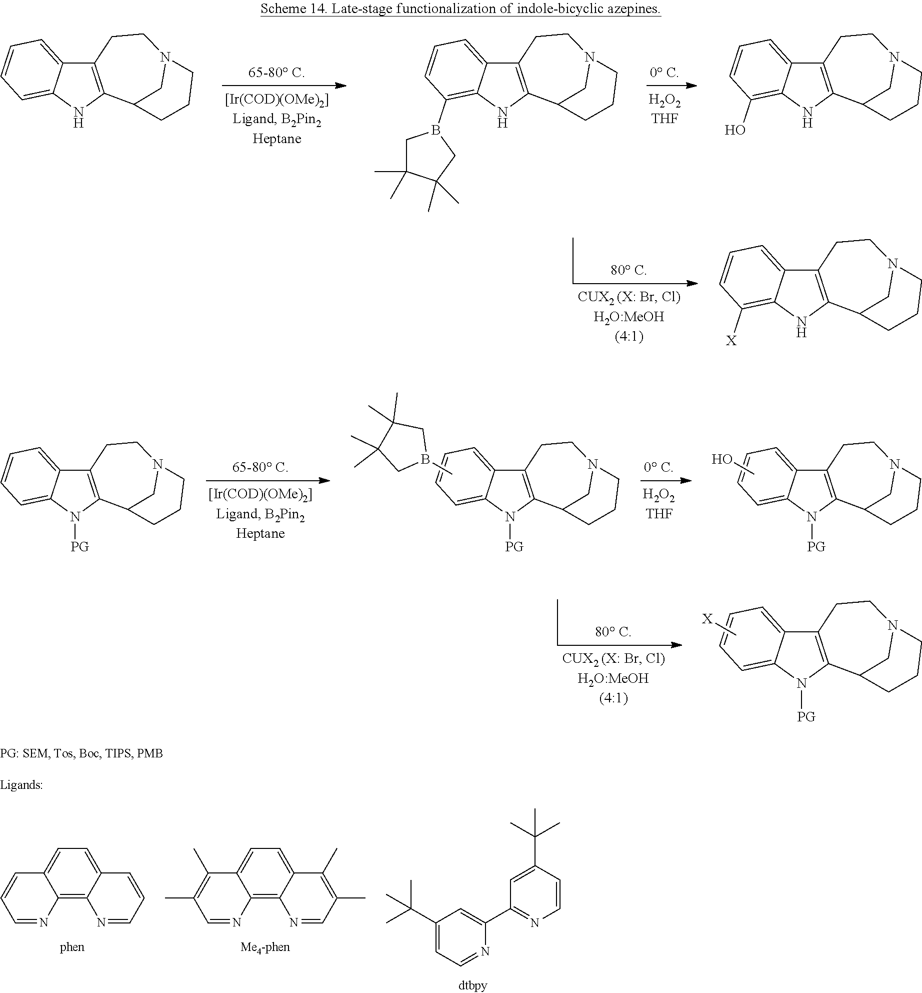

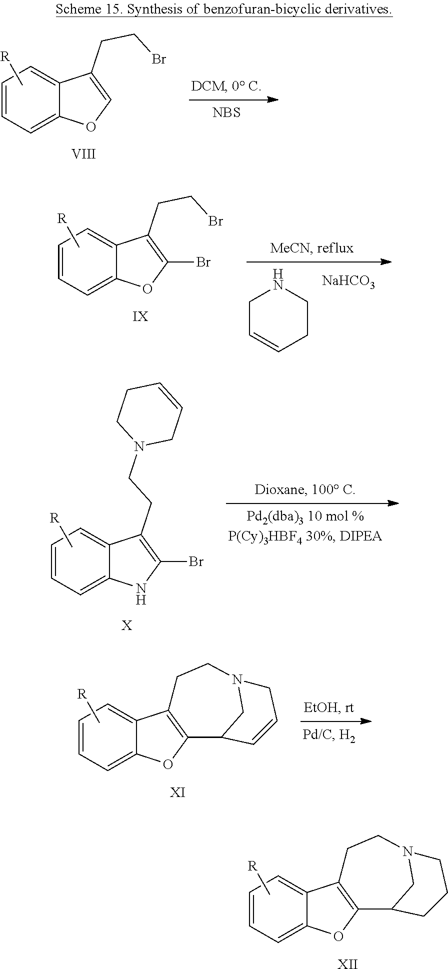

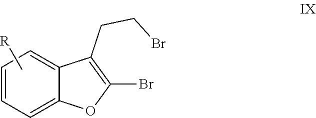

General procedure L: To a solution of substituted 3-(2-bromoethyl)benzofuran VIII (1 equiv) in DCM (0.3 M) at 0° C. was added NBS (0.95 equiv) dissolved in 1 M of DCE drop-wise via an addition funnel over 15 minutes. The reaction was quenched with a 10% sodium sulfite solution and the mixture was diluted with DCM. The contents of the flask were transferred to a separatory funnel and the layers were separated. The organics were washed with water, then brine, dried over anhydrous sodium sulfate, filtered and concentrated. The residue was taken up in minimal DCM and charged to a flash column chromatography.

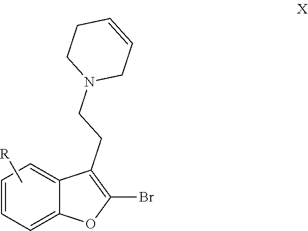

General procedure M: IX (1 equiv) and NaHCO₃ (4 equiv), were suspended anhydrous CH₃CN (0.125 M). 1, 2, 3, 6-Tetrahydropyridine (1.3 equiv) was added, and the resulting mixture was refluxed until TLC indicated the disappearance of the bromide (typically 1-2 days). The reaction was then diluted with H₂O, made strongly basic with aqueous NaOH, and extracted with CHCl₃ (3×). The combined organics were washed with H₂O, dried over Na₂SO₄, and concentrated to provide the crude product. The crude residue was purified with silica gel chromatography.

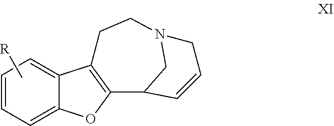

General procedure N: To a reaction tube under argon containing X (1 equiv) followed by P(Cy)₃HBF₄ (30 mol) and Pd₂(dba)₃ (15 mol) was added 1,4-Dioxane (0.1 M) followed by N,N-diisopropylethylamine (3 equiv). The mixture was heated to 100° C. for 6-12 hours with continuous stirring. The mixture was allowed to cool to ambient temperature, filtered over celite and concentrated in vacuo. The resulting material was subjected to flash chromatography.

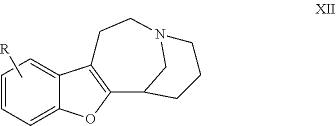

General procedure O: XI (1 equiv) was dissolved in ethanol (0.01 M) and 10% palladium on carbon (1 mg per mg of starting material) was added. The reaction mixture was kept under hydrogen atmosphere (40 psi) and was stirred at room temperature for 12 hours. Obtained mixture was filtered through celite and concentrated under reduced pressure. The resulting material was subjected to flash chromatography

### Example 62. 1,4,5,6,7,8-hexahydro-2H-3,7-methanoazonino[5,4-b]indol-11-ol 71

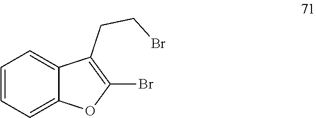 [SMILES: BrCCc1c(Br)oc2ccccc12]

General procedure L was applied using a solution of 3-(2-bromoethyl)benzofuran (1.5 g, 6.70 mmol, 1 equiv) in DCM (23.5 mL, 0.3 M) at 0° C. was added NBS (1.3 g, 6.36 mmol, 0.95 equiv) dissolved in 1 M of DCE. The resulting material was used directly for the next step without purification.

### Example 63. 1,4,5,6,7,8-hexahydro-2H-3,7-methanoazonino[5,4-b]indol-11-ol 72

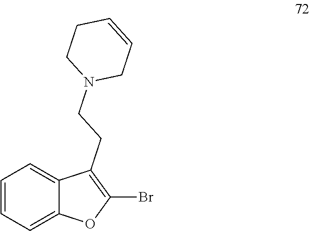 [SMILES: Brc1oc2ccccc2c1CCN1CC=CCC1]

General procedure M was applied using 71 (0.9 g, 2.96 mmol, 1 equiv) and NaHCO₃ (994 mg, 11.84 mmol, 4 equiv) in anhydrous CH₃CN (24 mL, 0.125 M) and 1,2,3,6-Tetrahydropyridine (0.4 mL, 4.44 mmol, 1.5 equiv). The resulting material was subjected to flash chromatography (98:2 EtOAc:TEA) to afford the title compound (717 mg, 94% yield) as light yellow oil. ¹H NMR (400 MHz, CDCl₃) δ 7.52-7.48 (m, 1H), 7.43-7.38 (m, 1H), 7.26-7.19 (m, 2H), 5.81-5.74 (m, 1H), 5.73-5.67 (m, 1H), 3.12-3.06 (m, 2H), 2.92-2.83 (m, 2H), 2.73-2.64 (m, 4H), 2.26-2.18 (m, 2H). ¹³C NMR (400 MHz, CDCl₃) δ 155.32, 128.65, 126.66, 125.29, 125.20, 124.17, 123.00, 118.82, 117.63, 110.99, 57.09, 52.68, 49.98, 26.25, 22.40. LRMS (EI) calcd. For C₁₅H₁₆BrNO 305.0 [M]⁺. found: 304.9.

### Example 64. 1,4,5,6,7,8-hexahydro-2H-3,7-methanoazonino[5,4-b]indol-11-ol 73

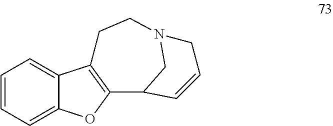 [SMILES: C1=CC2CN(C1)CCc1c2oc2ccccc12]

General procedure N was applied using 72 (710 mg, 2.32 mmol, 1 equiv) followed by P(Cy)₃HBF₄ (255 mg, 0.695 mmol, 30 mol %) and Pd₂ (dba)₃ (318 mg, 0.347 mmol, 15 mol %), 1,4-Dioxane (31 mL, 0.1 M) and N,N-diisopropylethylamine (1.2 mL, 6.96 mmol, 3 equiv). The resulting material was subjected to flash chromatography (98:2 DCM:MeOH) to afford the title compound (277 mg, 53% yield) as light brown gum. ¹H NMR (400 MHz, CDCl₃) δ 7.48-7.36 (m, 2H), 7.29-7.19 (m, 2H), 6.05-5.98 (m, 1H), 5.88-5.82 (m, 1H), 3.91-3.80 (m, 1H), 3.55-3.44 (m, 2H), 3.41-3.28 (m, 4H), 3.05-2.94 (m, 1H), 2.69-2.57 (m, 1H). ¹³C NMR (400 MHz, CDCl₃) δ 156.93, 153.53, 129.92, 126.18, 125.24, 123.01, 122.29, 118.16, 115.13, 110.86, 55.57, 50.50, 49.49, 34.15, 21.25. LRMS (EI) calcd. For C₁₅H₁₅NO 225.1 [M]⁺. found: 225.1.

### Example 65. 1,4,5,6,7,8-hexahydro-2H-3,7-methanoazonino[5,4-b]indol-11-ol 74

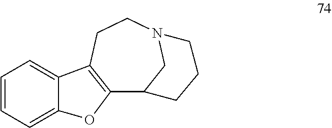 [SMILES: c1ccc2c3c(oc2c1)C1CCCN(CC3)C1]

General procedure 0 was applied using 73 (247 mg, 1.097 mmol, 1 equiv) was dissolved in ethanol (100 mL, 0.01 M) and 10% palladium on carbon (247 mg). The resulting material was subjected to flash chromatography (95:5 DCM:MeOH) to afford the title compound (125 mg, 50% yield) as white powder. ¹H NMR (500 MHz, MeOD) δ 7.54-7.50 (m, 1H), 7.42-7.39 (m, 1H), 7.31-7.22 (m, 2H), 3.77-3.70 (m, 1H), 3.68-3.63 (m, 2H), 3.59 (dd, J=13.7, 3.7 Hz, 1H), 3.51-3.46 (m, 1H), 3.42-3.38 (m, 2H), 3.26-3.14 (m, 2H), 2.15-2.07 (m, 1H), 2.01 (tt, J=13.4, 4.2 Hz, 1H), 1.93-1.81 (m, 1H), 1.74-1.67 (m, 1H). ¹³C NMR (500 MHz, MeOD) δ 154.36, 154.13, 128.81, 124.05, 122.42, 118.36, 114.62, 110.40, 54.51, 51.09, 50.27, 32.13, 26.20, 19.51, 17.10. LRMS (EI) calcd. For C₁₅H₁₇NO 227.1 [M]⁺. found: 227.1.

### Example 66. 1,4,5,6,7,8-hexahydro-2H-3,7-methanoazonino[5,4-b]indol-11-ol 75

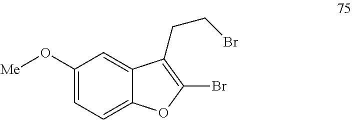 [SMILES: COc1ccc2oc(Br)c(CCBr)c2c1]

General procedure L was applied using a solution of 3-(2-bromoethyl)-5-methoxybenzofuran (1.0 g, 3.94 mmol, 1 equiv) in DCM (20.0 mL, 0.3 M) at 0° C. was added NBS (1.15 g, 3.74 mmol, 0.95 equiv) dissolved in 1 M of DCE. The resulting material was subjected to flash chromatography (97:3 Hexanes:ether) to afford the title compound (1.08 g, 82% yield) as light yellow oil. ¹H NMR (500 MHz, CDCl₃) δ 7.32 (dd, J=8.9, 1.9 Hz, 1H), 6.93 (s, 1H), 6.89-6.83 (m, 1H), 3.85 (s, 1H), 3.62-3.54 (m, 1H), 3.23-3.14 (m, 2H).

### Example 67. 1,4,5,6,7,8-hexahydro-2H-3,7-methanoazonino[5,4-b]indol-11-ol 76

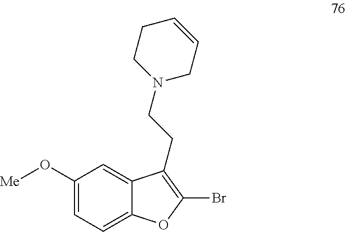 [SMILES: COc1ccc2oc(Br)c(CCN3CC=CCC3)c2c1]

General procedure M was applied using 75 (1.66 g, 4.97 mmol, 1 equiv) and NaHCO₃ (1.67 g, 19.88 mmol, 4 equiv) in anhydrous CH₃CN (40 mL, 0.125 M) and 1,2,3,6-Tetrahydropyridine (0.6 mL, 6.46 mmol, 1.3 equiv). The resulting material was subjected to flash chromatography (97:3 EtOAc:TEA) to afford the title compound (1.0 mg, 60% yield) as light yellow oil. ¹H NMR (300 MHz, CDCl₃) δ 7.29 (d, J=8.9 Hz, 1H), 6.97 (d, J=2.6 Hz, 1H), 6.84 (dd, J=8.9, 2.6 Hz, 1H), 5.83-5.65 (m, 2H), 3.83 (s, 3H), 3.14-3.04 (m, 2H), 2.92-2.80 (m, 2H), 2.75-2.62 (m, 4H), 2.35-2.16 (m, 2H). ¹³C NMR (300 MHz, CDCl₃) δ 156.13, 150.27, 129.15, 127.02, 125.28, 125.19, 117.70, 112.46, 111.45, 101.80, 57.02, 55.96, 52.70, 49.99, 26.26, 22.49. LRMS (EI) calcd. For C₁₆H₁₈BrNO₂ 335.1 [M]⁺. found: 335.1.

### Example 68. 1,4,5,6,7,8-hexahydro-2H-3,7-methanoazonino[5,4-b]indol-11-ol 77

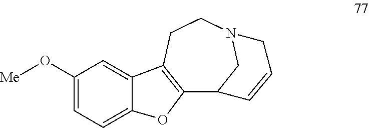 [SMILES: COc1ccc2oc3c(c2c1)CCN1CC=CC3C1]

General procedure N was applied using 76 (2.0 mg, 5.95 mmol, 1 equiv) followed by P(Cy)₃HBF₄ (655 mg, 1.78 mmol, 30 mol %) and Pd₂(dba)₃ (817 mg, 0.892 mmol, 15 mol %), 1,4-Dioxane (60 mL, 0.1 M) and N,N-diisopropylethylamine (2.68 mL, 14.87 mmol, 3 equiv). The resulting material was subjected to flash chromatography (95:5 DCM:MeOH) to afford the title compound (889 mg, 58% yield) as light brown gum. ¹H NMR (500 MHz, CDCl₃) δ 7.27 (d, J=7.9 Hz, OH), 6.85-6.78 (m, 2H), 3.97-3.90 (m, 1H), 3.83 (s, 3H), 3.59 (d, J=13.1 Hz, 1H), 3.54-3.41 (m, 3H), 3.41-3.30 (m, 2H), 3.00-2.91 (m, 1H), 2.76-2.68 (m, 1H). LRMS (EI) calcd. For C₁₆H₁₇NO₂ 255.1 [M]⁺. found: 255.1.

### Example 69. 1,4,5,6,7,8-hexahydro-2H-3,7-methanoazonino[5,4-b]indol-11-ol 78

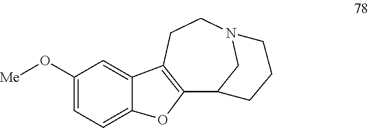 [SMILES: COc1ccc2oc3c(c2c1)CCN1CCCC3C1]

General procedure 0 was applied using 77 (889 mg, 3.48 mmol, 1 equiv) was dissolved in ethanol (150 mL, 0.02 M) and 10% palladium on carbon (889 mg). The resulting material was subjected to flash chromatography (90:10 DCM:MeOH) to afford the title compound (770 mg, 86% yield) as white powder. ¹H NMR (500 MHz, CDCl₃) δ 7.27 (d, J=7.8 Hz, 1H), 6.87 (d, J=2.6 Hz, 1H), 6.83 (dd, J=8.8, 2.6 Hz, 1H), 3.85 (s, 2H), 3.45-3.39 (m, 1H), 3.39-3.27 (m, 3H), 3.22-3.11 (m, 2H), 3.10-3.02 (m, 2H), 2.73 (dt, J=16.6, 3.4 Hz, 1H), 2.12-2.05 (m, 1H), 1.88 (tt, J=13.2, 4.2 Hz, 1H), 1.77-1.63 (m, 1H), 1.37 (d, J=14.0 Hz, 1H). LRMS (EI) calcd. For C₁₆H₁₉NO₂ 257.1 [M]⁺. found: 257.1.

### Example 70. 1,4,5,6,7,8-hexahydro-2H-3,7-methanoazonino[5,4-b]indol-11-ol 79

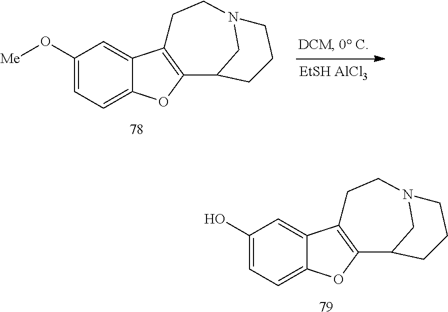

To a solution of 78 (25 mg, 0.097 mmol, 1 equiv) in dry dichloromethane (1 mL, 0.125 M) at 0° C. was added aluminum chloride (78 mg, 0.583 mmol, 6 equiv) followed by ethanethiol (0.13 mL, 1.75 mmol, 18 equiv), and the resulting mixture was allowed to warm to room temperature and stirred until TLC indicated the complete consumption of starting material (typically <1.5 h). The reaction was then quenched with saturated aqueous NaHCO₃ (100 mL per mmol of starting material) and extracted with DCM (4×-6×, until no further extraction by TLC). The combined organic layers were dried over Na₂SO₄ and concentrated to provide the crude product. The resulting material was subjected to flash chromatography (9:1 DCM/MeOH) to afford the title compound (18 mg, 76% yield) as light off white solid. ¹H NMR (500 MHz, MeOD) δ 7.15 (d, J=8.7 Hz, 1H), 6.79 (d, J=2.5 Hz, 1H), 6.68 (dd, J=8.7, 2.5 Hz, 1H), 3.39-3.33 (m, 1H), 3.27-3.20 (m, 2H), 3.11-3.01 (m, 4H), 2.73 (dt, J=16.8, 3.7 Hz, 1H), 2.06-2.01 (m, 1H), 1.95-1.84 (m, 1H), 1.78-1.67 (m, 1H), 1.38 (d, J=14.1 Hz, 1H). ¹³C NMR (500 MHz, MeOD) δ 158.30, 152.65, 148.39, 130.75, 114.47, 111.52, 110.19, 102.99, 54.01, 53.27, 49.15, 34.97, 29.06, 23.44, 20.37. LRMS (EI) calcd. For C₁₅H₁₇NO₂ 243.1 [M]l. found: 243.1.

### Example 71. Biological Characterization of Azepine Analogs

Potency (IC₅₀ and EC₅₀) and efficacy values for hERG, rKOR-CHO, hMOR-CHO, h5-HT2A-HEK, h5-HT2B-CHO, h5-HT2C HEK were obtained by screening the selected compounds via commercially available assay services.

TABLE 1
Background information for commercial
binding and functional assays.

| Ion Channel | | | | Reference |
|---|---|---|---|---|
| or Receptor | Species | Cell Line | Assay Type | Compound |
| hERG | Human | HEK-293 | Radioligand | Terfenadine |
| | | | ([3H]Dofetilide) | |
| | | | Displacement | |
| KOR | Rat | CHO | Agonist (cAMP) | U-50,488 |
| 5-HT2A | Human | HEK-293 | Agonist (IP₁) | Serotonin |
| 5-HT2B | Human | CHO | Agonist (IP₁) | Serotonin |
| 5-HT2C | Human | HEK-293 | Agonist (IP₁) | Serotonin |

Inhibition assays of transporters (hSERT and rVMAT2) were performed according to the following protocol.

Cell Culture Preparation and Maintenance

Stably transfected hSERT-HEK and rVMAT2-HEK cellular cultures were maintained in Dulbecco's Minimal Essential Medium (DMEM) with GlutaMAX (Gibco) with the following additions: 10% (v/v) Fetal Bovine Serum (FBS, Atlanta Biologicals), 100 U/mL Penicillin (Gibco), and 10 μg/mL Streptomycin (Gibco). With regards to the former cell lineage, an additional ingredient, 500 μg/mL Geneticin (G418) (Gibco) was included to preserve the respective transgene.

hSERT and rVMAT2 Fluorometric Screening Assays

For both hSERT and rVMAT2 screening experiments, respective singly transfected cells were seeded at a density of 0.09×10⁶ cells/well in poly-D-Lysine (Alamanda Polymers, Inc.) coated white solid-bottom 96-well plates (Costar). Growth was permitted for approximately 44 hours in said aqueous media and at an incubation environment of 37° C. and 5% Carbon Dioxide. At the beginning of the experiment, the cellular growth solution was aspirated, and individual cells were rinsed with 150 μL of 1×Dulbecco's Phosphate Buffered Saline (PBS; HyClone). 63 μL of Experimental Media (consisting of the following contents: DMEM without phenol red but with 4.5 g/L of D-Glucose (Gibco), 1% (v/v) FBS (Atlanta Biologicals), 100 U/mL Penicillin (Gibco), and 10 μg/mL Streptomycin (Gibco)) with 2×tiered concentrations of inhibitor (or DMSO, the vehicle of these experiments) were added to the respective wells. Control inhibitors used in these studies include Imipramine for hSERT experiments, and Reserpine for rVMAT2 experiments (Eiden, L. E. and Weihe, E. 2011; Sette, M. et al. 1983). At the conclusion of the pre-incubation period (60 minutes for hSERT experiments and 30 minutes for rVMAT2 experiments), 63 μL of Experimental Media containing 2×various concentrations of tested inhibitor (or vehicle) along with a specified amount of fluorescent substrate, APP⁺ (Karpowicz, R. J. et al 2013) (final concentration: 1.1 μM for hSERT experiments) or FFN206 (Hu, G. et al. 2013) (final concentration: 0.75 μM for rVMAT2 experiments) were added to the present solution contained within the wells. After a required incubation period (30 minutes for hSERT experiments and 60 minutes for rVMAT2 experiments) for proper fluorescent probe uptake, the contents of each well were aspirated and consequently, rinsed twice with 120 μL of PBS. A final solution of 120 μL of PBS is finally added to all corresponding wells for cell maintenance before undergoing fluorescence uptake reading by a BioTek H1MF plate reader. The excitation and emission wavelengths of APP⁺ were set at 389 and 442 nm, respectively. Alternatively, the excitation and emission wavelengths of FFN206 were designed at 370 and 464 nm, respectively.

G-Protein BRET Functional Opioid Assays

HEK-293T cells were obtained from the American Type Culture Collection (Rockville, MD) and were cultured in a 5% CO2 atmosphere at 37° C. in Dulbecco's Modified Eagle Medium (high glucose #11965; Life Technologies Corp.; Grand Island, NY) supplemented with 10% FBS (Premium Select, Atlanta Biologicals; Atlanta, GA), 100 U/mL penicillin, and 100 μg/mL streptomycin (#15140, Life Technologies). DNA Constructs: The mouse MOR (mMOR), the mouse DOR (mDOR) and the rat KOR (rKOR) were provided by Dr Lakshmi Devi at Mount Sinai School of Medicine. The G proteins used included untagged GαoB with Renilla luciferase 8 (RLuc8) inserted at position 91 (GαoB-RLuc8); Gβ1 (β1); Gγ2 which was fused to the full-length mVenus at its N-terminus via the amino acid linker GSAGT (mVenus-γ2). All constructs were sequence confirmed prior to use in experiments.

Transfection: The following cDNA amounts were transfected into HEK-293T cells (5×10⁶ cells/plate) in 10-cm dishes using polyethylenimine (PEI) in a 1:1 ratio (diluted in Opti-MEM, Life Technologies): 2.5 μg MOR/DOR/KOR, 0.125 μg GαoBRLuc8, 6.25 μg (1, 6.25 μg mVenus-γ2. Cells were maintained in the HEK-293T media described above. After 24 hours the media was changed, and the experiment was performed 24 hours later (48 hours after transfection). BRET: Transfected cells were dissociated and re-suspended in phosphate-buffered saline (PBS). Approximately 200,000 cells/well were added to a black-framed, white-well, 96-well plate (#60050; Perkin Elmer; Waltham, MA). The microplate was centrifuged and the cells were resuspended in PBS. After 5 minutes, 5 μM of the luciferase substrate coelenterazine H was added to each well. After 5 minutes, ligands were added and the BRET signal was measured 5 minutes later on a PHERAstar FS plate reader. The BRET signal was quantified by calculating the ratio of the light emitted by the energy acceptor, mVenus (510-540 nm), over the light emitted by the energy donor, RLuc8 (485 nm). This drug-induced BRET signal was normalized using the Emax of a known agonist (DAMGO, DPDPE or U-50,488) as the maximal response at MOR/DOR/KOR. Data were analyzed using the dose-response-stimulation nonlinear curve fitting model (log[agonist] vs. response (three parameters)).

Tail-Flick Test

C57BL/6J (8-12 weeks, 22-31 g) were purchased from the Jackson Laboratory (Bar Harbor, ME) and housed 5 mice per cage with food and water available ad libitum. Mice were maintained on a 12-hr light/dark cycle (lights on 7:00-19:00) and all testing was done in the light cycle. Temperature was kept constant at 22±2° C., and relative humidity was maintained at 50±5%. Mice were moved to the testing room 30 minutes before the experiment to allow for acclimation. The body weight of each mouse and base tail-flick value were recorded. Mice were administered a 1 mg/kg s.c. dose of compound solution (volume of injection 220-310 μL based on body weight). After injection mice were returned to the home cage and allowed to rest for 30 minutes. Thirty minutes post injection the tail-flick measurement was taken using thermal stimulation via IR on a Ugo Basile unit set to 52 PSU (ten seconds was used as a maximum latency to prevent tissue damage). Mice were then administered 3 mg/kg s.c. dose, allowed to rest for 30 minutes, followed by another tail-flick measurement. This process was repeated for doses 10 and 30 mg/kg in increasing order. Tail-flick latencies for the different doses were expressed as percentage of maximum potential effect (% MPE) by subtracting the experimental value by the base tail flick value then dividing by the difference between the maximum possible latency (10 seconds) and the base tail-flick value and finally multiplying by 100. All tail flick experiments were performed by an experienced blinded male experimenter.

Data Analysis

Numerical analysis of the collected experimental data preceded as accordingly. Respective inhibitor values were first subtracted from vehicular values to quantify the respective fluorescence uptake. This metric was then analyzed using the dose-response-inhibitor nonlinear curve fitting model ([inhibitor] vs response (three parameters)) as supplied by GraphPad Prism 8 software. For each inhibitor, the model supplied a respective IC₅₀±SEM value (Table 1). From this intermediate metric, calculation of the inhibition constant, K₁±SEM, was made possible using the Cheng-Prusoff Equation (Yung-Chi, C. and Prusoff, W. H. 1973) and the following established constants: Kₘ (for APP⁺)=1.6 μM (hSERT) and Kₘ (for FFN206)=1.2 μM (rVMAT2). It must be noted that the lower the Kᵢ value that is found, the greater the potency that the candidate inhibitor possesses at said transporter.

TABLE 2
IC₅₀ Values for hERG Binding Assay and EC₅₀ Values for KOR,
5-HT2A/2B/2C Agonist Assays of Selected Compounds. In parenthesis are
indicated % inhibition of control specific binding or % of control
agonist maximal response at 10 μM.

| Compound | IC₅₀ hERG | EC₅₀ KOR | EC₅₀ 5-HT2A | EC₅₀ 5-HT2B | EC₅₀ 5-HT2C |
|---|---|---|---|---|---|
| 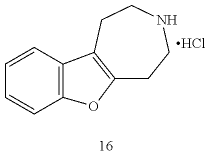 [SMILES: c1ccc2c3c(oc2c1)CCNCC3 ; Cl] | | ~1.2 μM (36%) | ~1 uM μM (77%) | >10 μM (20%) | <<0.1 μM (90%) |
| 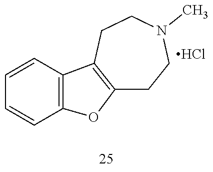 | >>10 μM (11%) | ~4.2 μM (36%) | >>10 μM (26%) | >>10 μM (4%) | ~0.2 μM (99%) |
| 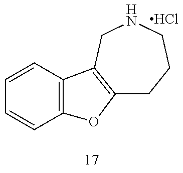 [SMILES: Cl ; c1ccc2c3c(oc2c1)CCCNC3] | | ~4.4 μM (45%) | ~60 μM (26%) | >10 μM (22%) | ~1.3 μM (78%) |
| 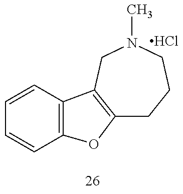 | | ~3.0 μM (47%) | >>10 μM (4%) | >>10 μM (3%) | ~14 μM (44%) |

TABLE 3
Selected Notable Azepine Analogs IC50 Values for hSERT and
rVMAT2 Transporters. All data are expressed in μM concentrations, and
represent the average values as calculated from experiments n ≥ 4,
with corresponding ± SEM.

| | SERT | |
|---|---|---|
| Compound | [μM ± SEM] | VMAT2 [μM ± SEM] |
| 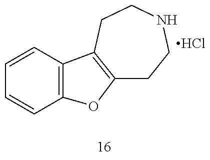 [SMILES: c1ccc2c3c(oc2c1)CCNCC3 ; Cl] | 0.45 ± 0.06 | 1.4 ± 0.23 |
| 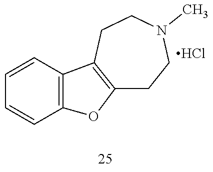 | 1.4 ± 0.30 | 4.9 ± 0.94 |
| 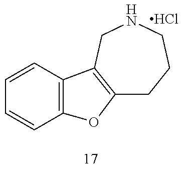 [SMILES: Cl ; c1ccc2c3c(oc2c1)CCCNC3] | 11 ± 2.9 | 0.061 ± 0.010 |
| 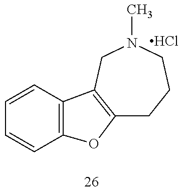 | 7.1 ± 2.3 | 15 ± 4.6 |

TABLE 4
IC₅₀ Values for hERG Binding Assay and EC₅₀ Values for KOR,
5-HT2A/2B/2C Agonist Assays of Selected Compounds. In parenthesis are
indicated % inhibition of control specific binding or % of control
agonist maximal response at 10 μM.

| Compound | IC₅₀ hERG | EC₅₀ 5-HT2A | EC₅₀ 5-HT2B | EC₅₀ 5-HT2C |
|---|---|---|---|---|
| 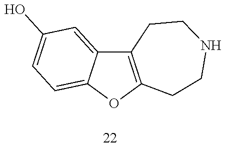 [SMILES: Oc1ccc2oc3c(c2c1)CCNCC3] | | 0.6 μM (72%) | >>10 μM (19%) | 0.1 μM (93%) |
| 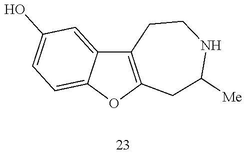 [SMILES: CC1Cc2oc3ccc(O)cc3c2CCN1] | | 1.9 μM (45%) | | 0.4 μM (86%) |
| 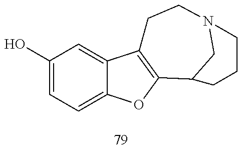 [SMILES: Oc1ccc2oc3c(c2c1)CCN1CCCC3C1] | ~60 μM (38%)^{a} | >>10 μM (1%) | 0.4 μM (5%) | >>10 μM (<0%) |

^{a}efficacy at 30 μM

TABLE 5
EC₅₀ Values for opioid receptor Agonist Assays of Selected
Compounds. In parenthesis are indicated % of control agonist maximal
response.

| | G Protein BRET Assay | | | CAMP Assay |
|---|---|---|---|---|
| Compound | EC₅₀ MOR | EC₅₀ DOR | EC₅₀ KOR | EC₅₀ KOR |
| 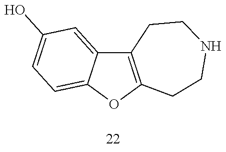 [SMILES: Oc1ccc2oc3c(c2c1)CCNCC3] | | | | >10 μM (<0% at 10 μM) |
| 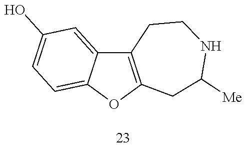 [SMILES: CC1Cc2oc3ccc(O)cc3c2CCN1] | 0.2 μM (16%) | 0.62 μM (45%) | 0.13 μM (36%) | 0.06 μM (96%) |
| 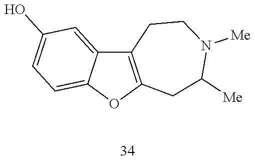 [SMILES: CC1Cc2oc3ccc(O)cc3c2CCN1C] | 0.32 μM (46%) | 1.2 μM (66%) | 0.079 μM (53%) | |
| 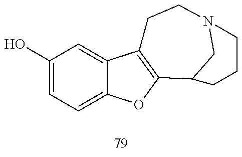 [SMILES: Oc1ccc2oc3c(c2c1)CCN1CCCC3C1] | 0.11 μM (44%) | 0.12 μM (55%) | 0.14 μM (30%) | 0.05 μM (91%) |

### DISCUSSION

Numerous iboga-type analogs are known (U.S. Pat. No. 9,988,377; U.S. application Ser. Nos. 14/240,681, 15/528,339; PCT International Application No. PCT/US2012/052327, PCT/US2015/062726). These analogs represent a further elaboration of the iboga skeleton to yield simpler and distinct structural systems with distinct pharmacology as well as improved side effects. The compounds described herein may be useful in treating opioid use disorder (OUD) and other SUDs, mood disorders, depression, and anxiety disorders, migraine and cluster headaches.

### REFERENCES

Alper, K. R. et al. Am J Addict 8, 234-242 (1999).
Alper, K. R. Ibogaine: A Review. Alkaloids Chem Biol 56, 1-38 (2001).
Belgers, M. et al. Transl Psychiatry 6, e826 (2016).
Clement, H. A. & Hall, D. G. Tetrahedron Lett. 59, 4334-4339 (2018).
Eiden, L. E. & Weihe, E. Ann N Y Acad Sci 1216(1), 86-98 (2011).
Gash, D. M. et al. Nature 380, 252-255 (1996).
Gonzalez, J. et al. Front. Pharmacol. 9, 374 (2018).
Glick, S. D. et al. Alkaloids Chem Biol 56, 39-53 (2001).
Glue, P. et al. The Journal of Clinical Pharmacology 55(6), 680-687 (2015).
Hartman, G. D. US20150225355A1, Aug. 13, 2015.
He, D-Y. et al. The Journal of Neuroscience 25(3), 619-628 (2005).
Ibrahem, I. & Córdova, A. Angewandte Chemie International Edition. 45, 1952-1956 (2006).
Karpowicz, R. J. et al. ACS Chem. Neurosci. 4(5), 858-869 (2013).
Kroupa, P. K. & Wells, H. MAPS, XV(2), 21-24 (2005).
Lançois, D. F. A. et al WO2016174079A1, Nov. 3, 2016.
Marton, S. et al. Front. Pharmacol. 10, 193 (2019).
Mash, D. C. et al. J Psychopharmacol 30, 688-697 (2016).
Mash, D. C. et al. Front Pharmacol 9, 529 (2018).
Matsumura, Y. & Oyama, T. JP2017100953A, Jun. 8, 2017.
Newman, S. G. et al. Chem. Commun. 5236-5238 (2009).
Schneider, J. A. & Sigg, E. B. Ann N Y Acad Sci 66, 765-776 (1957).
Schenberg, E. E. et al. J Psychopharmacol 28, 993-1000 (2014).
Sette, M. et al. Journal of Neurochemistry 40(3), 622-628 (1983).
Thoricatha, W. Psychedelic Times Jan. 28, 2020.
Wang, J. et al. Chem. Commun. 5144-5146 (2009).
Yung-Chi, C. & Prusoff, W. H. Biochemical Pharmacology 22(23), 3099-3108 (1973).
Zeidan, N. et al. Org. Lett. 19(19), 5058-5061 (2017).

What is claimed is:

1. A compound having the structure: 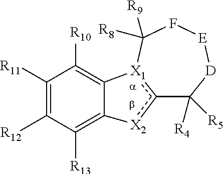
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,
or a pharmaceutically acceptable salt thereof.

2. The compound of claim 1 having the structure: 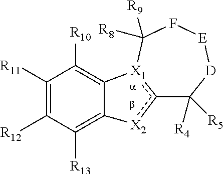
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON(alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and Ru is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then Ru is other than H, F or —CH₃, or
wherein X₁ is C or N;
X₂ is O, S, N or CR₁₅,
wherein R₁₅ is H, -(alkyl) or -cycloalkyl;
α and β represent a bond that is present or absent, and wherein either α or β is present,
wherein when α is present, then X₁ is C and X₂ is O or S, or
when β is present, then X₁ is N and X₂ is N or CR₁₅;
R₁ is H, or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid, or
wherein
X₁ is C or N;
X₂ is O, S or CR₁₅,
wherein R₁₅ is H, -(alkyl) or -cycloalkyl;
α and β represent a bond that is present or absent,
and wherein either α or β is present,
wherein when α is present, then X₁ is C and X₂ is O or S, or
when β is present, then X₁ is N and X₂ is CR₁₅;
R₁ is H or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and Ru is other than a ketone and a carboxylic acid, or
wherein
X₁ is C;
X₂ is O or S;
R₁ is H or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, or —OCF₃;
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is C, X₂ is S, and R₁, R₂, R₃, R₄, R₅, R₆, R₇, R₈, and R₉ are each H, and R₁₁ is Br, then D and E is other than NH,
or a pharmaceutically acceptable salt thereof, or
wherein
X₁ is C or N;
X₂ is N or NR₁₄,
wherein R₁₄ is H, -(alkyl) or -cycloalkyl;
α and β represent a bond that is present or absent, and
wherein either α or β is present,
wherein when α is present, then X₁ is C and X₂ is NR₁₄, or
when β is present, then X₁ is N and X₂ is N;
R₁ is H, or -(alkyl);
R₂, R₃, R₆, and R₇ are each independently H, -(alkyl), -(alkenyl), -(alkynyl), -cycloalkyl, -alkylcycloalkyl, -aryl, heteroaryl or -alkylaryl;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
when E is NR₁, then R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,
or a pharmaceutically acceptable salt thereof.

3-6. (canceled)

7. The compound of claim 1, wherein R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN, or
wherein R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂ (alkyl), —CONH₂, —CN, —CF₃, —CF₂H, —OCF₃ or —NO₂, or wherein R₁₀ and R₁₁ together form a —O(CH₂)O— or R₁₁ and R₁₂ together form a —O(CH₂)O— or
R₁₂ and R₁₃ together form a —O(CH₂)O—.

8-9. (canceled)

10. The compound of claim 2 having the structure: 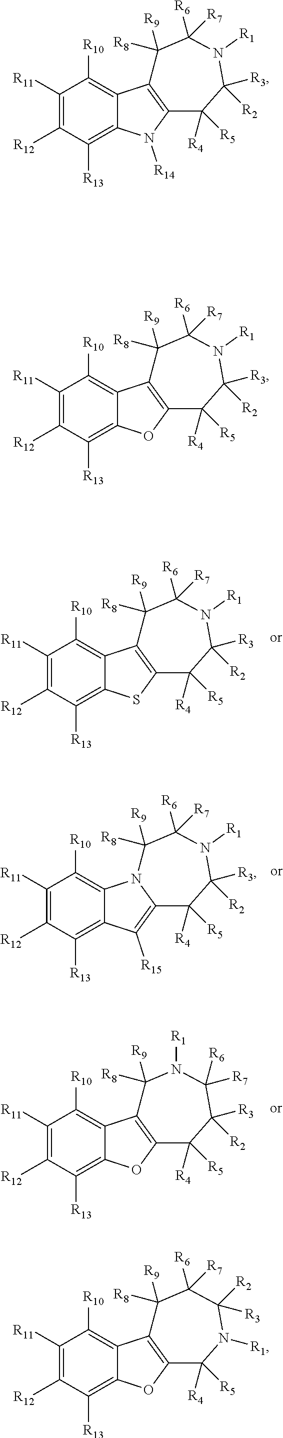
or a pharmaceutically acceptable salt thereof.

11. The compound of claim 10, wherein
R₁ is H or -(alkyl), or
R₁ is H, —CH₃ or —CH₂CH₅, or.
wherein R₄, R₅, R₈ and R₉ are each H, or
wherein R₄, R₅, R₆, R₇, R₈ and R₉ are each H, or
wherein R₂, R₃, R₄, R₅, R₈ and R₉ are each H, or
wherein R₂, R₃, R₄, R₅, R₆, R₇, R₈ and R₉ are each H.
wherein R₂, R₃, R₆ and R₇ are each independently H, -(alkyl), -alkylcycloalkyl or -alkylaryl, or
R₂, R₃, R₆, and R₇ are each independently H, —CH₃, —CH₂CH₃, —CH₂CH₂CH₃ or —CH(CH₃)₂, or
wherein R₁₀, R₁₁, R₁₂ and R₁₃ are each independently H, -(alkyl), —OH, —O(alkyl), —S(alkyl), —OAc, —CO₂(alkyl), —CF₃ or halogen, or
R₁₀, R₁₁, R₁₂ and R₁₃ are each independently H, —CH₃, —OH, —OCH₃, —SCH₃, —CF₃ or F, or,
wherein R₁ is H or —CH₃, or
wherein R₃, R₄, R₅, R₇, R₈ and R₉ are each H, or
wherein R₁ is H or —CH₃, and R₂ and R₆ are each independently H, —CH₃ or —CH₂CH₃, or
wherein R₂ and R₃ are each independently H, -(alkyl), -alkylcycloalkyl or -alkylaryl, or
R₂ and R₃ are each independently H, —CH₃, —CH₂CH₃, —CH₂CH₂CH₃ or —CH(CH₃)₂, or
wherein R₁₀, R₁₁, R₁₂ and R₁₃ are each independently H, -(alkyl), OH, —O(alkyl), —S(alkyl), OAc, —CO₂(alkyl), —CF₃ or halogen, or R₁₀, R₁₁, R₁₂, and R₁₃ are each independently H, —CH₃, —OH, —OCH₃, —SCH₃, —CF₃ or F, or
wherein R₁ is H or —CH₃, or
R₁ is H or —CH₃, R₂ is H, —CH₃ or —CH₂CH₃, and R₃ is H, or
wherein R₆ and R₇ are each independently H, -(alkyl), -alkylcycloalkyl or -alkylaryl, or
R₆ and R₇ are each independently H, —CH₃, —CH₂CH₃, —CH₂CH₂CH₃ or —CH(CH₃)₂, or,
wherein R₁₀, R₁₁, R₁₂ and R₁₃ are each independently H, -(alkyl), OH, —O(alkyl), —S(alkyl), OAc, —CO₂(alkyl), —CF₃ or halogen, or R₁₀, R₁₁, R₁₂ and R₁₃ are each independently H, —CH₃, OH, —OCH₃, —SCH₃, —CF₃ or F, or
wherein R₁ is H or —CH₃, or R₁ is H or —CH₃, R₆ is H, —CH₃ or —CH₂CH₃, and R₇ is H, or wherein R₁₀, R₁₁, R₁₂ and R₁₃ are each independently H, -(alkyl), OH, —O(alkyl), —S(alkyl), OAc, —CO₂(alkyl), —CF₃ or halogen, or R₁₀, R₁₁, R₁₂ and R₁₃ are each independently H, —CH₃, OH, —OCH₃, —SCH₃, —CF₃ or F.

12-29. (canceled)

30. The compound of claim 2,
wherein D is CR₂R₃, E is CR₆R₇ and F is NR₁, or
wherein X₁ is C and X₂ is NR₁₄, or X₁ is C and X₂ is O, or X₁ is C and X₂ is S, or X₁ is N and X₂ is CR₁₅, or X₁ is N and X₂ is N, or
wherein D is NR₁, E is CR₂R₃, and F is CR₆R₇, or
wherein R₁ is H or -(alkyl), or
R₁ is H, —CH₃ or —CH₂CH₅, or
wherein R₂, R₃, R₄ and R₅ are each independently H, -(alkyl), -alkylcycloalkyl or -alkylaryl, or
wherein R₆, R₇, R₈ and R₉ are each independently H, -(alkyl), -alkylcycloalkyl or -alkylaryl, or
R₆, R₇, R₈ and R₉ are each independently H, —CH₃, —CH₂CH₃, —CH₂CH₂CH₃ or —CH(CH₃)₂, or
wherein R₂, R₃, R₄, R₅, R₆, R₇, R₈ and R₉ are each H, or
wherein R₁₀, R₁₁, R₁₂ and R₁₃ are each independently H, -(alkyl), OH, —O(alkyl), —S(alkyl), OAc, —CO₂(alkyl), —CF₃ or halogen, or R₁₀, R₁₁, R₁₂ and R₁₃ are each independently H, —CH₃, OH, —OCH₃, —SCH₃, —CF₃ or F, or
wherein R₁ is H or —CH₃.

31-46. (canceled)

47. The compound of claim 1 having the structure: 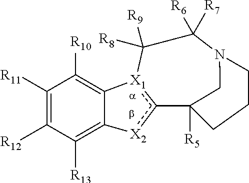
wherein
X₁ is C or N;
X₂ is O, S, N or NR₁₄,
wherein R₁₄ is H, -(alkyl) or -cycloalkyl;
α and β represent a bond that is present or absent, and wherein either α or β is present,
wherein when α is present, then X₁ is C and X₂ is O, S or NR₁₄, or
when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH₂)O— or
R₁₁ and R₁₂ together form a —O(CH₂)O— or
R₁₂ and R₁₃ together form a —O(CH₂)O—;
wherein when X₁ is C, X₂ is NR₁₄, and R₅, R₆, R₇, R₈, R₉, R₁₀, R₁₂, R₁₃ and R₁₄ are each H, then R₁₁ is other than H, F or —CH₃,
or a pharmaceutically acceptable salt or ester thereof.

48. The compound of claim 47 having the structure: 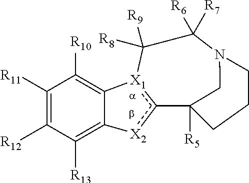
wherein
X₁ is C or N;
X₂ is O, S, N or NR₁₄,
wherein R₁₄ is H, -(alkyl) or -cycloalkyl;
α and β represent a bond that is present or absent, and wherein either α or β is present,
wherein when α is present, then X₁ is C and X₂ is O, S or NR₁₄, or
when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and R₅, R₆, R₇, R₈, R₉, R₁₀, R₁₂, R₁₃ and R₁₄ are each H, then Ru is other than H, F or —CH₃, or
or a pharmaceutically acceptable salt or ester thereof.

49. The compound of claim 48, wherein
X₁ is C or N;
X₂ is O, S or N;
α and β represent a bond that is present or absent, and wherein either α or β is present,
wherein when α is present, then X₁ is C and X₂ is O or S, or
when β is present, then X₁ is N and X₂ is N;
R₅, R₈ and R₉ are each independently H, -(alkyl), -(alkenyl), -alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl) or —CON (alkyl)₂;
R₆ and R₇ are each independently H, -(alkyl), -(alkenyl), -(alkynyl), -cycloalkyl, -alkylcycloalkyl, -aryl, heteroaryl or -alkylaryl; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃; or
R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN, or
wherein R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂ (alkyl), —CONH₂, —CN, —CF₃, —CF₂H, —OCF₃ or —NO₂, or
wherein R₁₀ and R₁₁ together form a —O(CH₂)O— or R₁₁ and R₁₂ together form a —O(CH₂)O— or R₁₂ and R₁₃ together form a —O(CH₂)O—,
or a pharmaceutically acceptable salt or ester thereof.

50-52. (canceled)

53. The compound of claim 47 having the structure: 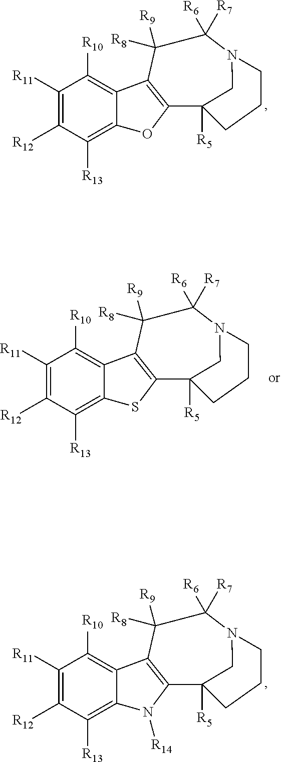
or a pharmaceutically acceptable salt or ester thereof.

54. The compound of claim 47, wherein R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN, or
wherein R₅ is H, -(alkyl), —OH, —O(alkyl), —OAc, —S(alkyl), —CO₂(alkyl), —CONH₂, —CONH(alkyl), —CON(alkyl)₂ or —CN, or
wherein R₅, R₈ and R₉ are each independently H, -(alkyl), -alkylcycloalkyl, -alkylaryl, —O(alkyl), —S(alkyl), —OAc, —CO₂(alkyl), and R₆ and R₇ are each independently H, -(alkyl), -alkylcycloalkyl or -alkylaryl, or
R₅, R₈ and R₉ are each independently H, —CH₃, —CH₂CH₃, —CH₂CH₂CH₃, —CH(CH₃)₂ or —CO₂Me and R₆ and R₇ are each independently H, —CH₃, —CH₂CH₃, —CH₂CH₂CH₃, —CH(CH₃)₂ or —CO₂Me, or
wherein R₅, R₆, R₇, R₈ and R₉ are each H, or
R₅, R₆ and R₇ are each H, or
R₅, R₈ and R₉ are each H, or
R₆ is —CH₃, and R₅, R₇, R₈ and R₉ are each H, or
wherein R₁₀, R₁₁, R₁₂, and R₁₃ are each independently H, -(alkyl), OH, —O(alkyl), —S(alkyl), OAc, —CO₂(alkyl), —CF₃ or halogen, or R₁₀, R₁₁, R₁₂ and R₁₃ are each independently H, —CH₃, OH, —OCH₃, —SCH₃, —CF₃ or F, or
R₁₀, R₁₁, R₁₂ and R₁₃ are each independently H, —CH₃, —CH₂CH₃, —CH(CH₃)₂, —OH, —OCH₃, —OCH₂CH₃, —SCH₃, —CF₃, F or Cl, or
wherein R₁₀, R₁₁, R₁₂ and R₁₃ are each independently H, —CH₃, —CH₂CH₃, —CH(CH₃)₂, cyclopropyl, —OH, —OCH₃, —OCH₂CH₃, —SCH₃, —CF₃, F, Cl or NO₂, or
wherein R₁₀ and R₁₁ together form a —O(CH₂)O—, R₁₁ and R₁₂ together form a —O(CH₂)O— or R₁₂ and R₁₃ together form a —O(CH₂)O—, or
wherein R₁₀, R₁₁, R₁₂ and R₁₃ are each H, or
R₁₀, R₁₂ and R₁₃ are H, and R₁₁ is OH, or
wherein R₁₀, R₁₂ and R₁₃ are H, and R₁₁ is —O (alkyl), or
R₁₀, R₁₂ and R₁₃ are H, and R₁₁ is —OCH₃.

55-62. (canceled)

63. The compound of claim 47 having the structure: 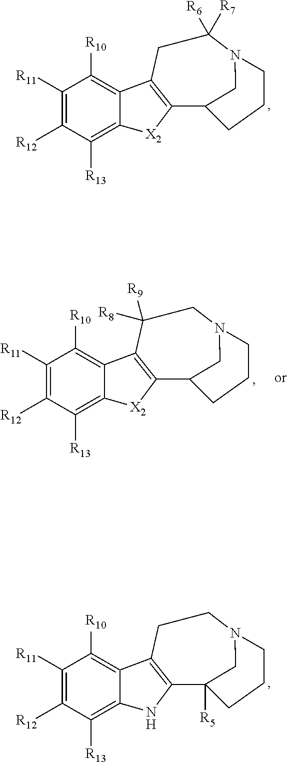
wherein X₂ is O, R₆ is —CH₃, R₇ is H, and R₁₁ is —OH, or X₂ is NR₁₄, R₆ is —CH₃, R₇ is H, and R₁₁ is —OH, wherein R₁₄ is H, or
wherein R₅ is H, -(alkyl), —OH, —O(alkyl), —OAc, —S(alkyl), CO₂ (alkyl), —CONH₂, —CONH (alkyl), —CON (alkyl)₂ or —CN, or
wherein R₅ is —CO₂Me, and R₁₀, R₁₁, R₁₂ and R₁₃ are each H, or R₅ is —CO₂Me, R₁₁ is OH, and R₁₀, R₁₂ and R₁₃ are each H, or R₅ is —CO₂Me, R₁₁ is —OCH₃, and R₁₀, R₁₂ and R₁₃ are each H
or a pharmaceutically acceptable salt or ester thereof.

64-69. (canceled)

70. The compound of claim 1 wherein the compound has the structure: 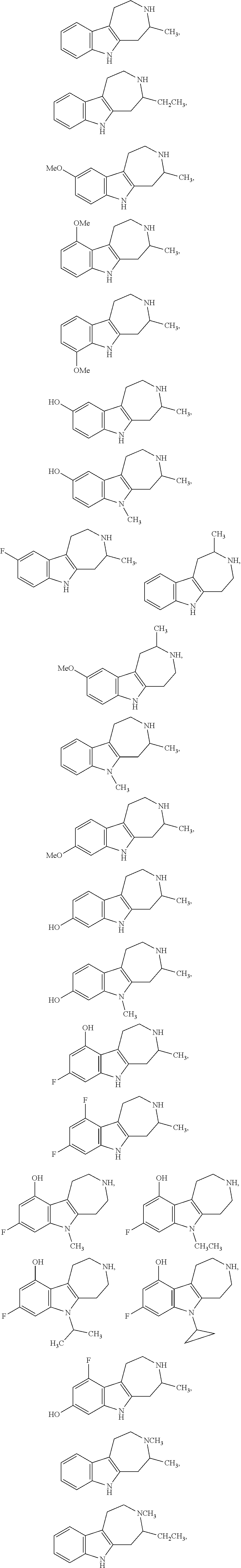 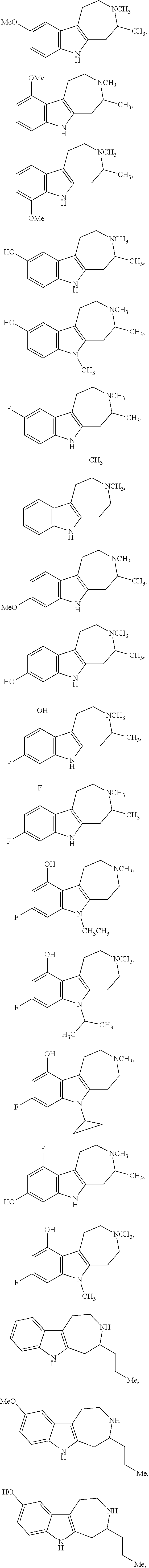 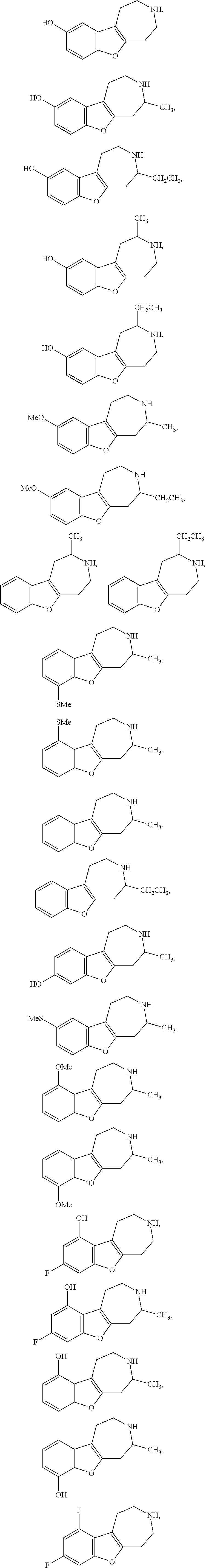 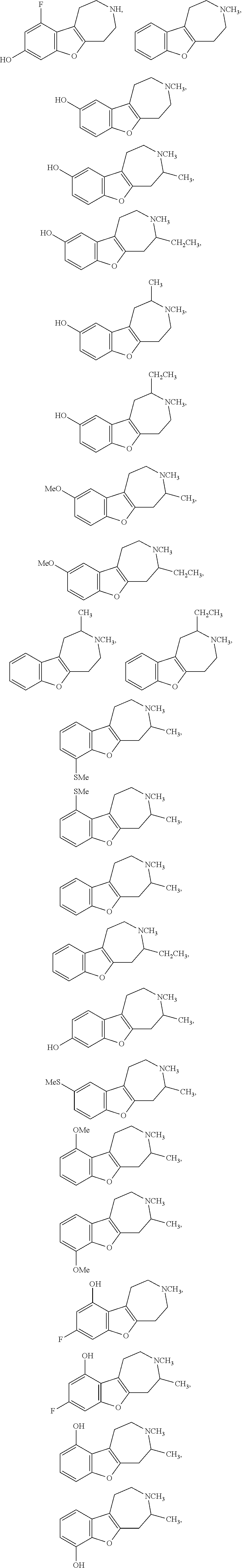 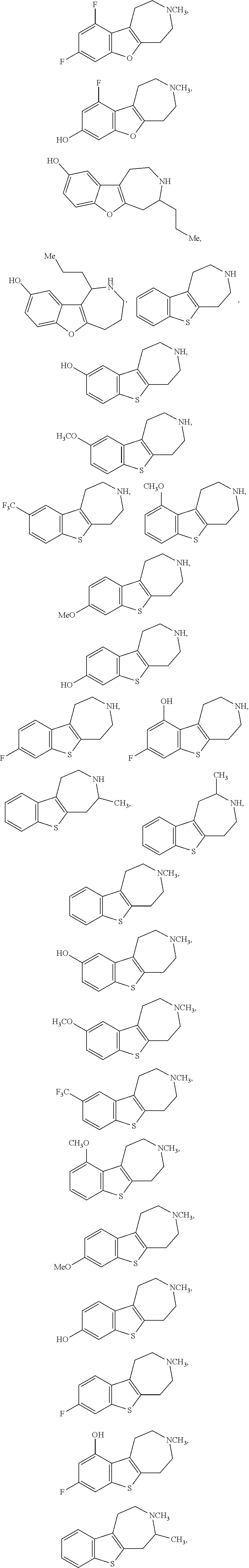 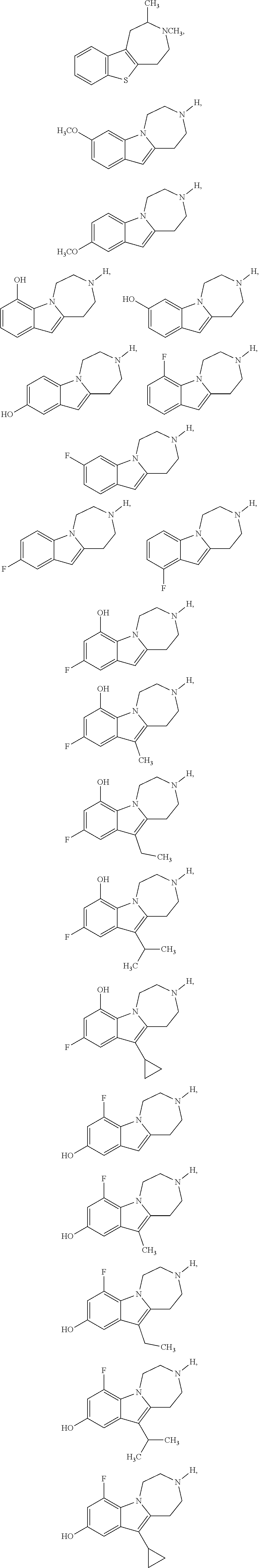 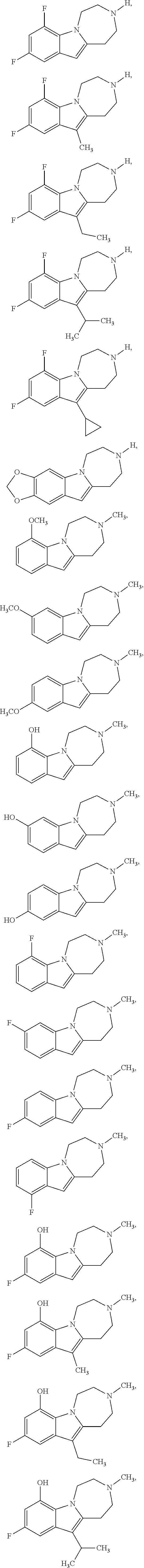 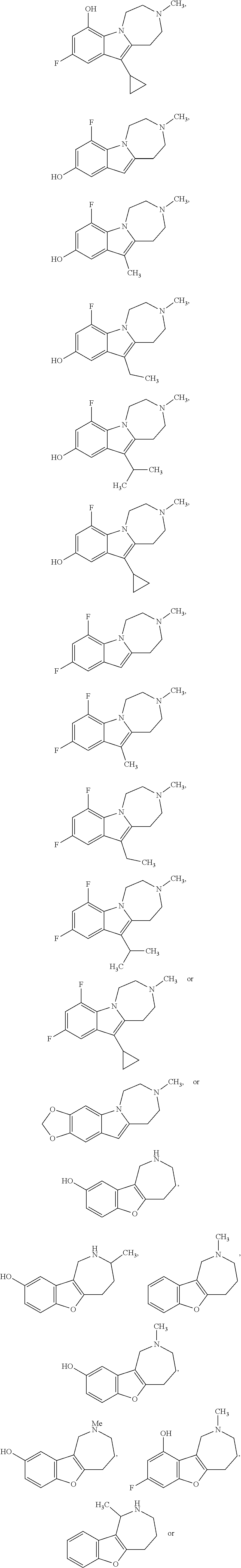 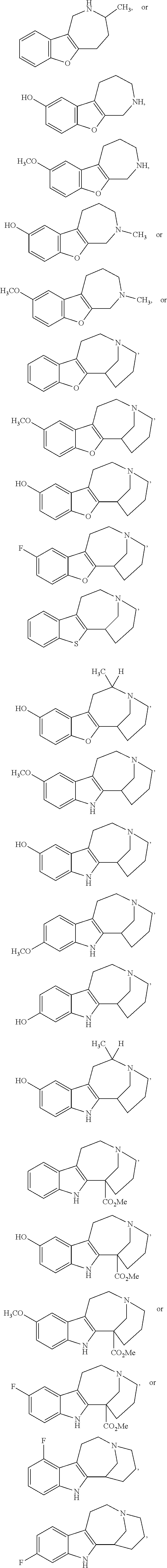 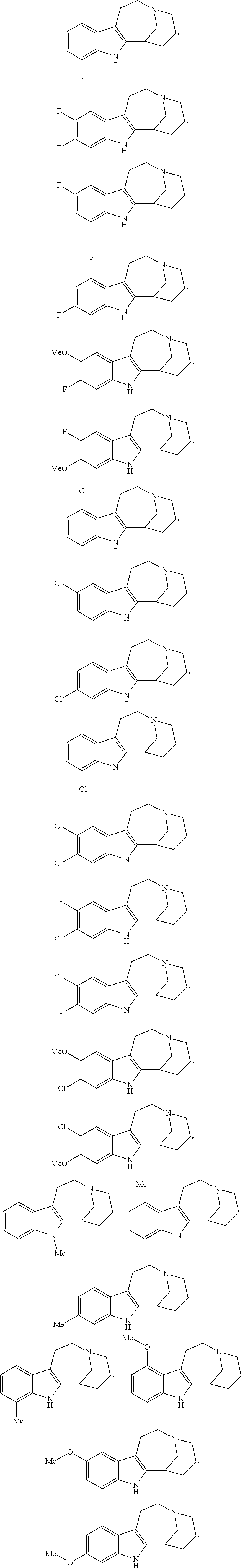 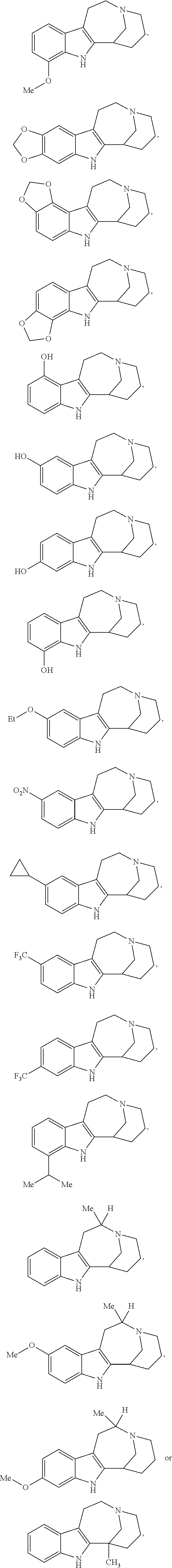
or a pharmaceutically acceptable salt thereof.

71. A pharmaceutical composition comprising the compound of claim 70 and a pharmaceutically acceptable carrier.

72. A pharmaceutical composition comprising a compound having the structure: 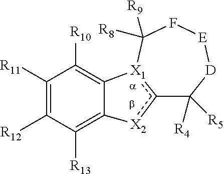
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH)O— or
R₁₁ and R₁₂ together form a —O(CH₂)O— or
R₁₂ and R₁₃ together form a —O(CH)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and Ru is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then Ru is other than H, F or —CH₃,
and a pharmaceutically acceptable carrier.

73. The pharmaceutical composition of claim 72, wherein the compound has the structure: 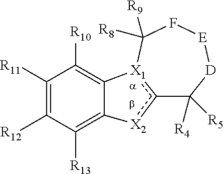
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or NO₂ or
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
wherein when X₁ is C, X₂ is O, and E is NH, NCH₃, NCH₂CH₃, or NCH(CH₃)₂, and one of R₁₀, R₁₁, R₁₂ and R₁₃ is —OCH₃ or —SCH₃, then (i) one of R₂, R₃, R₄, R₅, R₆, R₇, R₈ or R₉ is other than H, or (ii) at least two of R₁₀, R₁₁, R₁₂ and R₁₃ are other than H,
wherein when X₁ is C, X₂ is O, and F is NH, then at least one of R₂, R₃, R₄, R₅, R₆, R₇, R₈, R₉, R₁₀, R₁₁, R₁₂ or R₁₃ is other than H,
wherein when X₁ is N, X₂ is CR₁₅, D is CR₂R₃, E is NR₁, F is CR₆R₇, and R₁, R₂, R₃, R₄, R₅, R₆, R₇, R₈ and R₉ are H, and R₁₅ is H, then one of R₁₀, R₁₁, R₁₂ or R₁₃ is other than H, and R₁₀ is other than OMe, R₁₁ is other than Br, R₁₂ is other than Br and Cl, and R₁₃ is other than OMe,
wherein when X₁ is N, X₂ is CR₁₅, D is CR₂R₃, E is NR₁, F is CR₆R₇, R₁ is alkyl, R₂, R₃, R₄, R₅, R₆, R₇, R₈ and R₉ are H, and R₁₅ is CH₃, then at least one of R₁₀, R₁₁, R₁₂ or R₁₃ is other than H and CH₃, and R₁₁ is other than a ketone and a carboxylic acid,
wherein when R₁ and R₄ together form a —(CH₂)₃—, X₁ is C, X₂ is NR₁₄, D is CR₂R₃, E is NR₁, F is CR₆R₇, and R₂, R₃, R₅, R₆, R₇, R₈, R₉, R₁₀, R₁₂, R₁₃ and R₁₄ are each H, then R₁₁ is other than H, F or —CH₃,
or a pharmaceutically acceptable salt thereof.

74. A method of activating 5HT2A, 5HT2C, or both 5HT2A and 5HT2C receptors comprising contacting the 5HT2A and 5HT2C receptors with the composition of claim 71; or
a method of inhibiting SERT receptor comprising contacting the SERT receptor with the composition of claim 71; or
a method of activating kappa-opioid receptor comprising contacting the kappa-opioid receptor with the composition of claim 71; or
a method of inhibiting nicotinic acetylcholine receptor comprising contacting the nicotinic acetylcholine receptor with the composition of claim 71,
wherein the nicotinic acetylcholine receptor is α3β4, or
a method of treating a subject afflicted with substance use disorder comprising administering to the subject the composition of claim 71 comprising an effective amount of the compound, so as to thereby treat the subject afflicted with the substance use disorder,
wherein the substance use disorder is opioid use disorder, alcohol use disorder or stimulant use disorder, or
a method of treating a subject afflicted with opioid withdrawal symptoms comprising administering to the subject the composition of claim 71 comprising an effective amount of the composition, so as to thereby treat the subject afflicted with the opioid withdrawal symptoms, or
a method of altering the psychological state of a subject comprising administering to the composition of claim 71 comprising an effective amount of the composition, so as to thereby alter the psychological state of the subject, or
a method of enhancing the effect of psychotherapy in a subject comprising administering to the subject the composition of claim 71 comprising an effective amount of the composition, so as to thereby enhance the effect of the psychotherapy in the subject, or
a method of treating a subject afflicted with a depressive disorder, a mood disorder, an anxiety disorder, Parkinson's disease, or traumatic brain injury comprising administering to the subject the composition of claim 71 comprising an effective amount of the composition, so as to thereby treat the subject afflicted with the depressive disorder, the mood disorder, the anxiety disorder, Parkinson's disease or the traumatic brain injury, or
a method of treating a subject afflicted with a headache or a migraine comprising administering to the subject the composition of claim 71 comprising an effective amount of the composition, so as to thereby treat the subject afflicted with the headache or the migraine.

75-82. (canceled)

83. A method of treating a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy, comprising administering to the subject an effective amount of a compound having the structure: 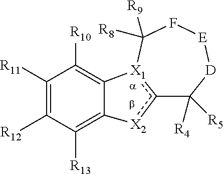
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), —CON(alkyl)₂ or —CN,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -cycloalkyl, -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H, —OCF₃ or —NO₂ or
R₁₀ and R₁₁ together form a —O(CH)O— or
R₁₁ and R₁₂ together form a —O(CH₂)O— or
R₁₂ and R₁₃ together form a —O(CH)O—;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H, or
wherein the compound has the structure: 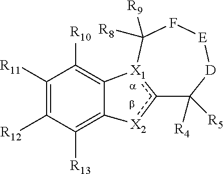
wherein
D, E and F are each independently NR₁, CR₂R₃ or CR₆R₇,
wherein one of D, E and F is NR₁ and the remaining two are CR₂R₃ or CR₆R₇,
wherein R₁ is H or -(alkyl), and
wherein R₂, R₃, R₆ and R₇ are each independently H, -(alkyl), -(alkenyl), -(alkynyl), -cycloalkyl, -alkylcycloalkyl, -aryl, heteroaryl or -alkylaryl;
X₁ is C or N;
X₂ is O, S, N, NR₁₄ or CR₁₅,
wherein R₁₄ is H, -(alkyl) or -cycloalkyl,
wherein R₁₅ is H, -(alkyl) or -cycloalkyl, and
wherein X₂ is other than N when X₁ is N;
α and β represent a bond that is present or absent, and wherein either α or β is present,
wherein when α is present, then X₁ is C and X₂ is O, S or NR₁₄, or
when β is present, then X₁ is N and X₂ is N or CR₁₅;
R₄, R₅, R₈ and R₉ are each independently H, -(alkyl), -(alkenyl), -(alkynyl), -cycloalkyl, -alkylcycloalkyl, -aryl, heteroaryl, -alkylaryl, —OH, —O(alkyl), —OAc, —S(alkyl), —NH₂, —NH(alkyl), —N(alkyl)₂, —COOH, —CO₂(alkyl), —CONH₂, —CONH(alkyl), or —CON(alkyl)₂,
wherein when D is NR₁ then R₄ and R₅ are each independently H, -(alkyl), -(alkenyl), -(alkynyl), -cycloalkyl, -alkylcycloalkyl, -aryl, heteroaryl or -alkylaryl,
wherein when F is NR₁ then R₈ and R₉ are each independently H, -(alkyl), -(alkenyl), -(alkynyl), -cycloalkyl, -alkylcycloalkyl, -aryl, heteroaryl or -alkylaryl or
R₁ and R₄ together form a —(CH₂)ₘ—, wherein m represents an integer from 2 to 4; and
R₁₀, R₁₁, R₁₂ and R₁₃ are each independently H, halogen, -(alkyl), -(alkenyl), -(alkynyl), -(aryl), -(heteroaryl), —OH, —OAc, —O(alkyl), —O-(alkenyl), —O-(alkynyl), —O-(aryl), —O-(heteroaryl), —SH, —S(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —CO₂(alkyl), —CONH₂, —CN, —CF₃, —CF₂H or —OCF₃;
wherein when X₁ is C, X₂ is NR₁₄, and D is CR₂R₃, E is NR₁, F is CR₆R₇, then (i) R₁₄ and at least two of R₁₀, R₁₁, R₁₂ and R₁₃ are other than hydrogen, or (ii) one of R₂, R₃, R₆ and R₇ is other than H,
or a pharmaceutically acceptable salt thereof, so as to thereby treat a subject afflicted with a substance use disorder, opioid withdrawal symptoms, a depressive disorder, a mood disorder, an anxiety disorder, Parkinson's disease, traumatic brain injury, a headache, a migraine, or of altering the psychological state or enhancing the effect of psychotherapy, comprising
activating 5HT2A, 5HT2C, or both 5HT2A and 5HT2C receptors, or
inhibiting SERT receptor, or
activating kappa-opioid receptor, or
inhibiting nicotinic acetylcholine receptor,
wherein the nicotinic acetylcholine receptor is α3β4.

84-86. (canceled)

87. The method of claim 83, wherein the substance use disorder is opioid use disorder, alcohol use disorder or stimulant use disorder,
wherein the compound has the structure: 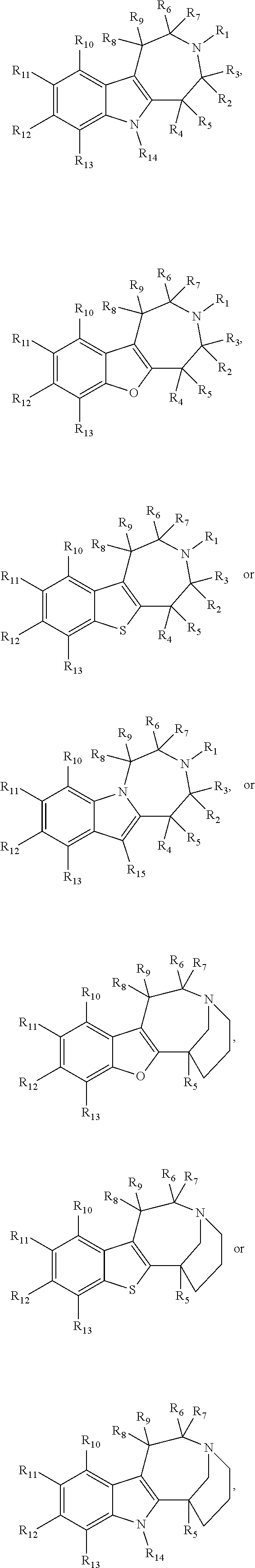
or a pharmaceutically acceptable salt thereof.

88-89. (canceled)

90. The method of claim 83, comprising
treating a subject afflicted with substance use disorder, wherein the substance use disorder is opioid use disorder, alcohol use disorder or stimulant use disorder or
treating a subject afflicted with opioid withdrawal symptoms, or
altering the psychological state of a subject, or
enhancing the effect of psychotherapy in a subject, or
treating a subject afflicted with a depressive disorder, a mood disorder, an anxiety disorder, Parkinson's disease, or traumatic brain injury, or
treating a subject afflicted with a headache or a migraine.

91-96. (canceled)
